(19) United States
(12) Patent Application Publication
(10) Pub. No.: US 2015/0353949 A1
(43) Pub. Date: Dec. 10, 2015

(54) METHOD FOR MODULATING PLANT ROOT ARCHITECTURE

(71) Applicants: The Australian National University, Canberra (AU); North Carolina State University, Raleigh, NC (US)

(72) Inventors: Michael Djordjevic, Kingston (AU); Nijat Imin, Kaleen (AU); David McKenzie Bird, Raleigh, NC (US); Peter Michael Digennaro, Raleigh, NC (US); Nadiatul Akmal Mohd Radzman, Canberra (AU)

(21) Appl. No.: 14/371,734

(22) PCT Filed: Jan. 11, 2013
(86) PCT No.: PCT/AU2013/000022

§ 371 Date: Jul. 10, 2014

(30) Foreign Application Priority Data
Jan. 11, 2012 (AU) 2012900109

(51) Int. Cl. C12N 15/82; A01N 37/46

(52) U.S. Cl. CPC C12N 15/8261; A01N 37/46; C12N 15/8227; C12N 15/8237

(57) ABSTRACT

The present invention provides a method for modulating or inhibiting modulation of the root architecture of a plant employing a new class of root architecture regulator (RAR) peptides, nucleotides encoding same, receptors therefor, binding agents thereof or agonists or antagonists thereof. Such methods encompass methods for increasing nutrient uptake by plants, increasing root nodule formation, promoting lateral root growth and development of plants, as well as methods for inhibiting root architecture modification resulting from root knot nematode action on plant roots. Also provided are RAR peptides, polynucleotides encoding RAR peptides and vectors and host cells comprising same, and plants obtained by methods of the invention or comprising RAR-encoding transgenes.

### FIELD OF THE INVENTION

The present invention relates to methods and materials for modulating the architecture of plant roots and thereby modulating the ability of plants to take up nutrients and/or water from soil. The invention also relates to methods and materials for treating or preventing root knot nematode attack of plant roots.

### BACKGROUND TO THE INVENTION

Root architecture is an important agronomic trait governed by a poorly understood integration of nascent lateral organ development programs with nutritional and environmental parameters. To effectively adapt to variations in the soil environment, root systems evolved two important features: developmental plasticity and a wide range of molecular systems to sense and respond to various soil parameters. The availability of nitrogen is a major determinant of root architecture. A complex but poorly understood interplay occurs between environmental cues and the innate root development pathways. Collectively this interplay dictates the optimization of root architecture and influences plant productivity and fitness.

There is therefore a need for a better understanding of root architecture and factors involved in this to develop plants with root architecture allowing for improved or more efficient nutrient uptake, or for otherwise better adapting plants to the environment they are to be grown in.

### SUMMARY OF THE INVENTION

The present investigations have shown that the root architecture of plants, including nodule formation and competency thereof, can be modulated by a family of peptides herein referred to as root architecture regulatory (RAR) peptides. It has also been found that root knot nematodes (RKNs) also express and secrete peptides of this family, thereby affecting the architecture of affected roots. This knowledge may provide a route for controlling the effects of root knot nematode attack on plant roots. Furthermore, in the course of these studies it has also been found that root knot nematodes also express and secrete other plant-like regulatory peptides, including root growth factors (RGFs), CLV3/ESR-related peptides (CLEs) and Inflorescence Deficient in Abscission peptides (IDAs).

Thus, according to an aspect of the invention, there is provided a method for modulating or inhibiting modulation of the root architecture of a plant, said method comprising:

(a) contacting the root zone of said plant with a root architecture regulator peptide (RAR), an analogue thereof or an RAR signaling agonist; or

(b) contacting the root zone of said plant with a RAR signaling antagonist; or

(c) introducing at least one mutation or exogenous nucleic acid into one or more plant cells which results in modulated RAR expression by a root knot nematode or by cells of a plant regenerated from or comprising said one or more plant cells; or

(d) introducing at least one mutation or exogenous nucleic acid into one or more plant cells which results in modulated RAR receptor expression by cells of a plant regenerated from or comprising said one or more plant cells; or

(e) introducing at least one mutation or exogenous nucleic acid into one or more plant cells which results in modulated affinity of one or more RARs for their respective RAR receptors, which modulated affinity arises through modifications in the RAR(s), RAR receptor(s) or in both expressed RAR(s) and RAR receptor(s); or

(f) introducing at least one exogenous nucleic acid into one or more plant cells which results in expression of one or more root knot nematode RAR binding agents or antagonists.

According to an embodiment of this aspect, the present invention provides a method for modulating lateral root development by a plant relative to an untreated or wild-type plant, comprising contacting the root zone of said plant with at least one RAR or introducing at least one exogenous RAR-encoding nucleic acid into one or more plant cells which results in increased RAR expression by cells of a plant regenerated from or comprising said one or more plant cells.

According to another embodiment of this aspect, the present invention provides a method for promoting lateral root development of a plant relative to an untreated or wild-type plant, comprising contacting the root zone of said plant with a RAR antagonist or introducing at least one mutation or at least one exogenous nucleic acid into one or more plant cells which at least one mutation or nucleic acid results in:
(i) decreased expression of one or more RARs, decreased expression of one or more RAR receptors, or decreased expression of one or more RARs and one or more RAR receptors by cells of a plant regenerated from or comprising said one or more plant cells, wherein said decreased expression of said RAR(s) or RAR receptor(s) occurs under conditions which would otherwise promote expression of said RAR(s) or RAR receptor(s); or
(ii) reduced affinity of one or more RARs for their respective RAR receptors, which reduced affinity arises through modifications in the RAR(s), RAR receptor(s) or in both expressed RAR(s) and RAR receptor(s) expressed by cells of a plant regenerated from or comprising said one or more plant cells.

According to another embodiment of this aspect, the present invention provides a method for inhibiting modulation of the root architecture of a plant by the action of a root knot nematode, comprising: contacting the root zone of a plant with a root knot nematode RAR signaling antagonist or binding agent; introducing into one or more plant cells at least one exogenous nucleic acid which suppresses, or the product of which suppresses expression of a root knot nematode RAR-encoding nucleic acid (such as a microRNA or siRNA or a microRNA-encoding or siRNA-encoding sequence); introducing into one or more plant cells at least one mutation or exogenous nucleic acid which results in modulated affinity of one or more root knot nematode RARs for plant RAR receptors, which modulated affinity arises through modifications in the plant RAR receptor(s); or introducing into one or more plant cells one or more exogenous nucleic acids which results in expression by said cell(s) of one or more root knot nematode RAR binding agents or antagonists.

According to another embodiment of this aspect, the present invention provides a method for inhibiting modulation of the root architecture of a plant by the action of a root knot nematode, comprising: contacting the root zone of a plant with a root knot nematode RGF signaling antagonist or binding agent; introducing into one or more plant cells at least one exogenous nucleic acid which suppresses, or the product of which suppresses expression of a root knot nematode RGF-encoding nucleic acid (such as a microRNA or siRNA or a microRNA-encoding or siRNA-encoding sequence); introducing into one or more plant cells at least one mutation or exogenous nucleic acid which results in modulated affinity of one or more root knot nematode RGFs for plant RGF receptors, which modulated affinity arises through modifications in the plant RGF receptor(s); or introducing into one or more plant cells one or more exogenous nucleic acids which results in expression by said cell(s) of one or more root knot nematode RGF binding agents or antagonists.

In view of the discovery that RKNs express a number of plant signaling molecules, including RARs, RGFs, IDAs and CLEs, and appear to deploy them to subvert normal plant root growth to their needs, it is postulated that another method for controlling damage by RKNs to plants may comprise inhibiting expression of peptidases or proteases responsible for cleaving signal peptides from the translated pre-secretion precursors of the regulatory proteins/peptides secreted by RKNs.

According to another embodiment of the invention, there is therefore provided a method for inhibiting modulation of the root architecture of a plant by the action of a root knot nematode, said method comprising introducing at least one exogenous nucleic acid into one or more plant cells which suppresses, or the product of which suppresses expression of a component of a root knot nematode signal peptidase complex.

Plants obtained by the methods outlined above, and plant parts (including leaves, stems, roots, tubers, flowers, fruit, seeds and parts thereof) are also provided.

According to another aspect, the present invention provides an isolated root architecture regulator peptide (RAR) or an analogue thereof, with the proviso that said RAR is not an Arabidopsis CEP1 peptide and said RAR-encoding nucleic acid is not an Arabidopsis CEP1 encoding sequence. According to an embodiment, the RAR is a root knot nematode RAR.

According to another aspect, the present invention provides an isolated polynucleotide comprising a nucleotide sequence coding for a RAR, with the proviso that said polynucleotide is not the Arabidopsis CEP1 encoding sequence, or a portion thereof. The polynucleotide may also comprise a secretion signal peptide sequence. According to an embodiment, the RAR-encoding sequence is a root knot nematode RAR-encoding sequence.

According to another aspect, the present invention provides an isolated polynucleotide comprising a nucleotide sequence coding for a root knot nematode RGF, or a portion thereof.

Also provided are vectors, transgenic cells, including plant cells, and transgenic plants, and seeds thereof comprising polynucleotides according to the invention.

Also contemplated by the present invention are methods for screening plants or root not nematodes for RARs, RAR receptor(s) and encoding sequences, and isolation of such peptides and encoding sequences. Further contemplated are methods for screening root knot nematodes for RGFs and encoding sequences, and isolation of such peptides and encoding sequences. Such screening methods may identify additional RARs, RGFs, as well as RAR or RGF receptors, encoding sequences and associated regulatory regions with altered properties (including altered affinities or altered nutrient responses).

Screening methods may comprise the use of polyclonal or monoclonal antibodies raised against one or more of the RARs or RGFs disclosed herein, labeled RAR(s) and RGF(s), or oligonucleotide probes or primer pairs based on one or more of the RAR-encoding or RGF-encoding sequences disclosed herein.

### BRIEF DESCRIPTION OF THE DRAWINGS

FIG. 1—RAR peptide ligands occur in higher plants and RKNs. (A) Weblogo plots show the 15 AA RAR peptides each with particularly strong C-terminal conservation. Angiosperm and RKN RAR peptides show strong similarity. Gymnosperm RAR-like peptides exhibit divergence at the amino-terminus with a highly conserved leucine instead of proline at position 7. Unlike dicots, monocot RARs do not contain F at position 2. (B) Putative MtRAR1 protein sequence. The amino-terminal signal sequence is blue and the conserved RAR peptides are red. Non-conserved sequences are green. (C, D) Putative MhRAR1 and MiRAR1 proteins. Two forms of RARs exist: the first has sequences flanking the RAR peptide (e.g. MhRAR1, C) and the second has no flanking sequences (e.g. MiRAR1; D).

FIG. 2—MtRAR gene expression is up-regulated by low N (nitrogen) and high CO₂ levels and is developmentally regulated during lateral organogenesis in M. truncatula. (A) Eight of 11 RAR genes were significantly up-regulated after four days growth on a N-free medium compared to plants grown with 0.5 mM NH₄NO₃. The expression of MtRAR3 and MtRAR10 was not included; they were not detected by qRT-PCR. N≧12; student's t-test P<0.05; **: P<0.01; ***: P<0.001. Bars indicate standard deviations. (B) Relative expression of MtRAR genes in response to high and low levels of nitrate and atmospheric CO₂. MtRAR1-2, 5-8 and 11 are significantly up-regulated in the roots after 14 days growth under low nitrate (0.25 mM KNO₃) and high CO₂ (800 ppm) compared to the roots of similar plants grown under high nitrate (5 mM KNO₃) and ambient CO₂. Plants grown at 0.25 mM KNO₃ were supplemented with 4.75 mM KCl to standardize potassium levels. MtRAR1 and MtRAR11 are up-regulated by low nitrate and MtRAR1 and MtRAR2 are up-regulated by high CO₂ even in high the presence of high nitrate suggesting independent regulation of RAR by CO₂ and nitrate. N≧12. (C). MtRAR1 expression is not affected by 24 h hormone treatments. (D) Microarray analysis of the MtRAR1 in different tissues. MtRAR1 is expressed in N-deprived roots and suppressed during all stages of nodule development (http://mtgea.noble.org/v2). Root (no nitrogen): grown under no nitrogen condition for four days; Nod: nodules; dpi: days post-inoculation with S. meliloti. (E-N) ProMtRAR1:GUS expression in the transgenic roots. (E and F) Nitrogen starvation for 4 days. (I and M) grown on 5 mM KNO₃. (J-L) Transverse sections at the root tip of N-starved plants; indicative locations of the sections are shown by the red lines in E. (G and N) MtRAR1:GUS roots inoculated with S. meliloti for one (G) or two weeks (N). (H) A cross-section of nodule induced by Sm 1021 at two weeks. Scale bars: 100 μM in E-G, I, M and N; 50 μM in H and J-L.

FIG. 3—Multiple root architecture phenotypes in M. truncatula caused by MtRAR1 over-expression or synthetic (putative) ligand addition. (A) Control root showing normal root hair development. (B-C) periodic humps (arrows) caused by MtRAR1 over-expression. (D-E) Periodic bumps (arrows) caused by synthetic MtRAR1 or MhRAR2 peptide respectively. (F-G) Root nodule formation on transgenic roots with the empty vector or MtRAR1 over-expression respectively. GFP is visible in C, F and G. (H) MtRAR1 over-expression caused periodic bumps in the GH3:GUS reporter construct line. GH3:GUS expresses strongly at bump sites and arrested lateral roots can be seen at two of the four sites (asterisk). A section of the control GH3:GUS root is shown in K and sections of two bump sites bearing arrested lateral roots are in L and M. Bumps are typified by circumferential cell proliferation (“CCP”) and this term is used interchangeably throughout. Asterisk, arrested lateral root; double asterisk, strong vascular GH3:GUS expression (I) Lateral root number is repressed six-fold by MtRAR1 over-expression. Histogram showing a comparison of lateral root numbers formed on the transgenic roots containing MtRAR1ox to an empty vector control. N≧319; ***: P<0.001. Bars indicate standard deviations. (J) The relative expression of nitrogen-signaling related genes MtAGL1, MtNRT2.5 and MtLBD38 are modulated by MtRAR1 over-expression in the transgenic roots. N≧12; *: P<0.05; ***: P<0.001. Scale bars: 100 μM in A-H; 50 μM in L-M.

FIG. 4—Periodic bumps are sites of localized cell division or contain arrested lateral roots. (A) Confocal imaging of a non-bump site on transgenic roots over-expressing MtRAR1. (B-C) CCP sites showing altered cell divisions occurring in epidermal, cortical and pericycle cells. Central nuclei are seen in C. (D) Bump site with arrested lateral root showing divided but un-differentiated cells. (E-F) Root diameter and the numbers of cortical cells are significantly increased at bump (CCP) sites (P<0.05). Scale bars: 50 μM.

FIG. 5—Shows a sequence analysis of RAR domains in higher plants and the RKNs. (A) Cladistic representation of RAR genes in plants. The RAR domains are shown for each evolutionarily significant Glade only. (B) Phylogenetic analysis of RAR domains in Meloidogyne hapla. (C) Phylogenetic analysis of RAR domains in higher plants and RKNs. (D) Alignment of RKN RARs. Blue box indicates signal sequence. Red box indicates RAR domain. For (B) and (C) sequence alignment of pro-RAR and C-terminal domains was done by a combination of ClustalW and manual adjustment. Phylogenic tree construction was done by MrBayes. Numbers report the posterior probabilities of the 50% majority consensus tree.)

FIG. 6—Amino acid sequence alignment of 11 putative RAR-coding genes in M. truncatula. All 11 sequences have predicted signal peptides at the N-terminus (boxed in blue), an intervening variable region of little or no sequence conservation and 15 amino acid long conserved region(s) close to the C-terminus end. RAR domains are boxed in red. Note some RARs have more than one RAR domain (e.g. MtRAR10 has four RAR peptide motifs whereas MtRAR 7, 9 and 1 each have two).

FIG. 7—Phenotypic changes caused by MtRAR1 over-expression in. M. truncatula transgenic roots. (A) The qRT-PCR results show that the expression of MtRAR1 is significantly elevated in transgenic roots containing the MtRAR1ox construct compared to transgenic roots containing the empty vector (control n≧).9; student's t-test ***: P<0.0001. Error bars indicate standard deviations. (B) Quantification of periodic CCP site formation in MtRAR1ox transgenic roots. The results of double blind scoring for the presence of periodic CCP site formation are shown. N?_{—} 300, Student's t-test ***: P<0.001. (C) MtRAR1ox increases number of nodules when inoculated with S. meliloti. N≧20; student's t-test *: P<0.05. Error bars indicate standard deviations. (D-E) MtRAR1ox reduces lateral root numbers. Compared to transgenic roots containing the empty vector control (D), fewer lateral roots are formed in transgenic roots bearing MtRAR1ox (E). Periodic CCP on MtRAR1 ox roots are indicated by asterisk. Scale bar=1 cm. (F-G) MtRAR1ox reduces root hair length and numbers when grown under agar. Fewer and shorter root hairs are formed in transgenic roots bearing MtRAR1ox (F) compared to transgenic roots containing the empty vector control (G). Scale bar=0.1 cm.

FIG. 8—Effect of a concentration gradient of MtRAR1 synthetic peptide on lateral root formation and root growth in M. truncatula. (A). Lateral root formation is significantly reduced on A17 plants grown on F medium containing 25 mM KNO₃ and 10⁻⁶M NA peptide (MtRAR1 domain one peptide). (B) Main root growth is significantly inhibited at 10⁻⁶M DIA peptide. t-test ***: P<0.001. Error bars indicate standard deviations.

FIG. 9—Phylogenetic tree of MADS-box homologues in Arabidopsis and M. truncatula. Sequence alignment was done by CLUSTAL and adjusted by hand. Phylogeny was created using MrBayes, with two chains running for 500000 generations, sampled every 500. A mixed amino acid model was used. Burn in was 250 data points. The above phylogeny represents the 50% majority consensus tree. Numbers are posterior probabilities.

FIG. 10—Phylogenetic tree of NRT homologues in Arabidopsis and M. truncatula. Sequence alignment was done by CLUSTAL and adjusted by hand. Phylogeny was created using MrBayes, with two chains running for 500000 generations, sampled every 500. A mixed amino acid model was used. Burn in was 250 data points. The above phylogeny represents the 50% majority consensus tree. Numbers are posterior probabilities.

FIG. 11—Alignment of the M. hapla putative RGF with RGFs from A. thaliana. The active domain region (boxed in red) is well conserved between the species.

FIG. 12—Putative IDA of M. hapla aligned with IDA and IDA-Like proteins from A. thaliana. In M. hapla, the signal sequence (blue box) is immediately followed by an IDA-Like domain (red box).

FIG. 13—Shows the 5-prime upstream region of the M. truncatula RAR1 gene, including the promoter region and the first 86 nucleotides of the RAR1-encoding region.

FIG. 14—Shows the effect of RAR peptide application on lateral root formation of the plants grown in jars. M. truncatula, M. sativa, Subterranean Clover Cultivar Woogenellup and white clover (Trifolium repens) plants were grown in 0.4% agar-containing magenta jars supplemented with F media with or without peptides for two weeks. The media contained 5 mM KNO₃. MtRAR1 peptide: FQ(P)TTPGNS(P)GVGH where (P) is hydroxyproline. Students’ t-test *: P<0.05; **: P<0.01; ***: P<0.001; N>12. Error bars represent SE.

FIG. 15—Shows the effect of RAR peptide application on lateral root formation of M. sativa plants. The plants were grown in 0.4% agar-containing magenta jars supplemented with F media with or without peptides for two weeks. The medium contained 5 mM KNO₃. MtRAR1 peptide: FQ(P)TTPGNS(P)GVGH where (P) is hydroxyproline.

FIG. 16—Shows the effect of RAR peptide application on lateral root formation of subterranean clover cultivar Woogenellup plants. The plants were grown in 0.4% agar-containing magenta jars supplemented with F media with or without peptides for two weeks. The media contained 5 mM KNO₃. MtRAR1 peptide: FQ(P)TTPGNS(P)GVGH where (P) is hydroxyproline.

FIG. 17—Shows the effect of RAR peptide application on lateral root formation of white clover (Trifolium repens) plants. The plants were grown in 0.4% agar-containing magenta jars supplemented with F medium with or without peptides for two weeks. The media contained 5 mM KNO₃. MtRAR1 peptide: FQ(P)TTPGNS(P)GVGH where (P) is hydroxyproline.

FIG. 18—Shows the effect of RAR peptide application on lateral root formation on microtom tomatoes. The plants were grown in 0.8% agar-containing plates supplemented with F media with or without peptides for two weeks. The media contained 5 mM KNO₃. MtRAR1 peptide: FQ(P)TTPGNS(P)GVGH where (P) is hydroxyproline. Students' t-test, **: P<0.01; N>12. Error bars represent SE.

FIG. 19—Shows the effect of RAR peptide application on lateral root formation on microtom tomatoes. The plants were grown in 0.8% agar-containing plates supplemented with F media with or without peptides for two weeks. The medium contained 5 mM KNO₃. MtRAR1 peptide: FQ(P)TTPGNS(P)GVGH where (P) is hydroxyproline.

FIG. 20—Shows the effect of RAR peptide application on root formation of soybean. The plants were grown in liquid supplemented with F medium with or without peptides for two weeks. The medium contained 5 mM KNO₃. GmRAR peptide: NFR(P)TAPGHS(P)GVGH where (P) is hydroxyproline. Students' t-test, **: P<0.01; N>6. Error bars represent SE.

FIG. 21—Shows the effect of RAR peptide application on root formation of soybean. The plants were grown in liquid supplemented with F medium with or without peptides for two weeks. The media contained 5 mM KNO₃. GmRAR peptide: NFR(P)TAPGHS(P)GVGH where (P) is hydroxyproline.

FIG. 22—Shows the effect of RAR peptide application on root formation of rice plants. The plants were grown in liquid media containing half strength MS media with or without peptides for two weeks. Monocot RAR peptide: DTRATDPGHS(P)GAGH where (P) is hydroxyproline. Students' t-test, *: P<0.05; N>6. Error bars represent SE.

FIG. 23—Shows the effect of RAR peptide application on root formation of rice plants. The plants were grown in liquid media containing half strength MS media with or without peptides for two weeks. Monocot RAR peptide: DTRATDPGHS(P)GAGH where (P) is hydroxyproline.

FIG. 24—Shows the phenotypes of 12 day old. WT Arabidopsis plants treated with 1 μM of RAR peptide.

FIG. 25—Shows the effect of RAR peptide addition on primary root in Arabidopsis.

FIG. 26—Shows the effect of RAR peptide addition on lateral root number in Arabidopsis.

FIG. 27—Shows the RAR time course showing primary root length.

FIG. 28—Shows the RAR time course showing lateral root number.

FIG. 29—Shows the effect of RAR on the number of lateral roots can be released by removing RAR exposure.

FIG. 30—Shows the effect of RAR on primary root length can be released.

FIG. 31—MtRAR1 over-expression increases nodulation and promotes nodule development at different nitrate concentrations: (a) Comparison of nodulation between MtRAR1 over-expressing plants and control plants at different temperatures when inoculated with S. meliloti strain 1021; (b) Partial nitrate tolerance of nodule development induced by WSM1022 on MtRAR1 over-expressing plants; (c) Acetylene reduction assay results; (d) Inoculation of plants with preformed CCP sites led to root nodule formation at the CCP sites at 25 mM KNO₃; (e) Nodule sections from RAR1 peptide-treated and untreated plants grown at either 0, 5 mM or 25 mM KNO₃ for three weeks.

FIG. 32—M. truncatula plants grown in 0.8% agar-containing plates supplemented with F media with or without peptides.

### DEFINITIONS

As used herein, the term “comprising” means “including principally, but not necessarily solely”. Variations of the word “comprising”, such as “comprise” and “comprises”, have correspondingly similar meanings.

As used herein the term “gene”, refers to a defined region that is located within a genome and that may comprise regulatory, nucleic acid sequences responsible for the control of expression, i.e., transcription and translation of the coding portion. A gene may also comprise other 5′ and 3′ untranslated sequences and termination sequences: Further elements that may be present are, for example, introns and coding sequences.

As used herein, the term “analogue” in the context of a peptide or protein means an artificial or natural substance that resembles the peptide or protein in function. For example, an RAR analogue will bind an RAR receptor and thereby bring about the same or similar result as if a natural RAR had bound to the receptor. In an embodiment such analogues may also resemble the RAR peptide in structure. Analogues contemplated in an embodiment of the present invention include fully or partially peptidomimetic compounds as well as peptides or proteins resembling a subject peptide in activity but comprising addition, deletion, or substitution of one or more amino acids compared to the subject peptide or protein. The term “analogue” as used herein with reference to nucleotide sequences encompasses sequences comprising addition, deletion, or substitution (including conservative amino acid substitutions) of one or more bases relative to a subject nucleotide sequence, wherein the encoded polypeptide resembles the polypeptide encoded by the subject nucleic acid molecule in function.

As used herein, the term “homologue” in the context of proteins means proteins having substantially the same functions and similar properties in different species, and which, within at least regions, share at least 50% amino acid identity. Such homologous proteins may share, over their entire amino acid sequences, at least about 30% amino acid identity, at least about 40% amino acid identity, at least about 50% amino acid identity, at least about 60% amino acid identity, at least about 70% amino acid identity, at least about 80% amino acid identity, at least about 90% amino acid identity or at least about 95% identity. Similarly, homologues of nucleic acid molecules are nucleic acid molecules that encode proteins having substantially the same functions and similar properties in different species, wherein the encoded proteins share, within at least regions, at least 50% amino acid identity (such nucleic acid homologues may share significantly less than 50% identity due to degeneracy in the genetic code, and differences in preferred codon usage amongst different genuses and species), and may share at least about 30% amino acid identity, at least about 40% amino acid identity, at least about 50% amino acid identity, at least about 60% amino acid identity, at least about 70% amino acid identity, at least about 80% amino acid identity, at least about 90% amino acid identity or at least about 95% identity over the whole encoded amino acid sequences.

“Conservative amino acid substitutions” refer to the interchangeability of residues having similar side chains. For example, a group of amino acids having aliphatic side chains includes glycine, alanine, valine, leucine, and isoleucine; a group of amino acids having aliphatic-hydroxyl side chains includes serine and threonine; a group of amino acids having amide-containing side chains includes asparagine and glutamine; a group of amino acids having aromatic side chains includes phenylalanine, tyrosine, and tryptophan; a group of amino acids having basic side chains includes lysine, arginine, and histidine; and a group of amino acids having sulfur-containing side chains includes cysteine and methionine. Typically, conservative amino acids substitution groups are: valine-leucine-isoleucine, phenylalanine-tyrosine, lysine-arginine, alanine-valine, and asparagine-glutamine. Typically, conservative amino acid substitution(s) will result in a protein or polypeptide retaining at least some of the biological activity of the protein or polypeptide without such a conservative amino acid substitution. More typically, conservative amino acid substitution(s) will result in a protein or polypeptide having substantially the same, or at least comparable biological activity as the protein or polypeptide without such a conservative amino acid substitution. Conservative amino acid substitution(s) may result in proteins or polypeptides having greater biological activity than the protein or polypeptide without such a conservative amino acid substitution.

The term “antibody” refers to an immunoglobulin molecule able to bind to a specific epitope on an antigen, and which may be comprised of a polyclonal mixture, or be monoclonal in nature. Antibodies may be entire immunoglobulins derived from natural sources, or from recombinant sources. An antibody according to the present invention may exist in a variety of forms including, for example, whole antibody, an antibody fragment, or another immunologically active fragment thereof, such as a complementarity determining region. Similarly, the antibody may be an antibody fragment having functional antigen-binding domains, that is, heavy and light chain variable domains. The antibody fragment may also exist in a form selected from the group consisting of: Fv, F_{ab}, F(ab)₂, scFv (single chain Fv), dAb (single domain antibody), hi-specific antibodies, diabodies and triabodies.

The term “isolated” indicates that the material in question has been removed from its naturally existing environment, and associated impurities reduced or eliminated. Essentially, the ‘isolated’ material is enriched with respect to other materials extracted from the same source (ie., on a molar basis it is more abundant than any other of the individual species extracted from a given source), and preferably a substantially purified fraction is a composition wherein the ‘isolated’ material comprises at least about 30 percent (on a molar basis) of all macromolecular species present. Generally, a substantially pure composition of the material will comprise more than about 80 to 90 percent of the total of macromolecular species present in the composition. Most preferably, the ‘isolated’ material is purified to essential homogeneity (contaminant species cannot be detected in the composition by conventional detection methods) wherein the composition consists essentially of the subject macromolecular species.

As used herein, the term “agonist” in the context of a peptide, polypeptide or protein refers to a molecule that binds with a receptor for that peptide, polypeptide or protein to trigger a physiological response usually triggered by the peptide, polypeptide or protein when it binds to said receptor. For example, a RAR agonist is a molecule that binds to a RAR receptor to trigger a root architectural response.

As used herein, the term “antagonist” in the context of a peptide, polypeptide or protein refers to a substance that interferes with the physiological response usually triggered by the peptide, polypeptide or protein when it binds to said receptor, or which interferes with binding of said peptide, polypeptide or protein to its receptor. For example, a RAR antagonist may be a substance that binds to a RAR or a RAR receptor to inhibit interaction between the RAR and the RAR receptor, which interaction would trigger a root architectural response. RAR antagonists may include antibodies to RARs or RAR receptors.

As used herein, the term “mutation” means any change in a polypeptide or nucleic acid molecule relative to a wild-type polypeptide or nucleic acid molecule from which the ‘mutant’ is derived and may, for example, comprise single or multiple amino acid or nucleotide changes, or both nucleotide and amino acid changes, including point mutations, null mutations, frame-shift mutations, and may comprise deletions, or insertions, or substitutions of one or more nucleic acids or amino acids, which may comprise naturally or non-naturally occurring nucleotides or amino acids or analogues thereof.

A “nucleic acid”, as referred to herein, refers to deoxyribonucleotides or ribonucleotides and polymers thereof in either single-, double-stranded or triplexed form. The term may encompass nucleic acids containing known analogues of natural nucleotides having similar binding properties as the reference nucleic acid. A particular nucleic acid sequence may also implicitly encompass conservatively modified variants thereof (e.g. degenerate codon substitutions) and complementary sequences. The terms “nucleic acid”, “nucleic acid sequence” or “polynucleotide” may also be used interchangeably with gene, cDNA, and mRNA encoded by a gene.

The terms “polypeptide”, “peptide” and “protein” may be used interchangeably herein to refer to a polymer of amino acid residues. Included within the scope of these terms are polymers in which one or more amino acid residues may comprise artificial chemical analogue(s) of corresponding naturally occurring amino acid(s), as well as, or instead of naturally occurring amino acid polymers. The terms “polypeptide”, “peptide” and “protein” may also include polymers including modifications, including post-translational modifications, such as, but not limited to, glycosylation, lipid attachment, sulfation, phosphorylation, gamma-carboxylation of glutamic acid residues, hydroxylation and ADP-ribosylation.

The term “primer” as used herein means a single-stranded oligonucleotide capable of acting as a point of initiation of template-directed DNA synthesis. An “oligonucleotide” is a short nucleic acid, typically ranging in length from 2 to about 500 bases. The precise length of a primer will vary according to the particular application, but typically ranges from 15 to 30 nucleotides. A primer need not reflect the exact sequence of the template but must be sufficiently complementary to hybridize to the template.

Within the scope of the terms “protein”, “polypeptide”, “polynucleotide” and “nucleic acid” as used herein are fragments and variants thereof, including but not limited to reverse compliment and antisense forms of polynucleotides and nucleic acids.

### DETAILED DESCRIPTION OF THE INVENTION

Cell-to-cell communication mechanisms coordinate cellular proliferation and differentiation in plants. Recently, new signal molecules have emerged that preside over the positional information required to co-ordinate plant growth. Amongst these are growth regulating peptides that act primarily as extracellular signals. Growth regulating plant peptides regulate all aspects of plant growth and development. The CLE (CLAVATA3/EMBRYO SURROUNDING REGION-related) peptides are well understood: different classes of plant CLEs regulate the differentiation and renewal of stem cell and control the developmental competency of legume roots for root nodule formation. Similarly, root growth factors (RGFs) are regulatory peptides that maintain the stem cell niche and transit cell proliferation.

An Arabidopsis gene, AtCEP1 (C-terminal encoded peptide), encoding a 14 or 15 amino acid secreted ligand has been previously described and reported to influence primary root growth (Ohyama K, Ogawa M, and Matsubayashi Y (2008), “Identification of a biologically active, small, secreted peptide in Arabidopsis by in silico gene screening, followed by LC-MS-based structure analysis”, The Plant Journal 55(1):152-160). AtCEP1 corresponds to AtRAR1 according to the nomenclature used herein.

In the present studies, a number of RAR peptides have been identified across a broad range of plant families (angiosperms and gymnosperms), and some of these characterised. Phylogenetic and genetic tools were used to examine the distribution and function of this multigene family, and analyses indicate that this family of genes is unique to higher plants and, surprisingly, occur in root knot nematode (RKN) genomes. Generally, these genes encode secreted peptides that contain 14-15 amino acid long conserved domains. It has been found that in the legume Medicago truncatula (barrel medic) these genes are regulated by N-limited conditions and elevated CO₂ and control the expression of genes (MtAGL1, MtNRT2.5 and MtLBD38) likely to coordinate N-status and uptake. Over-expression studies were used to demonstrate that one member of this gene family, designated as MtRAR1, which encodes two 15 amino acid peptides of this family, affects multiple aspects of root architecture and development including lateral root, nodule and root hair development, as well as shoot to root ratio. Multiple periodic bumps (CCP sites) form on roots at the expense of lateral root emergence and confocal imaging corroborates that these bumps share features with RKN-induced galls. Results also revealed RAR ligand mimicry by RKNs suggesting a role for these nematode peptides in gall/knot formation, including hyperplasia of cortical, epidermal and pericycle cells. Our results also revealed RAR ligand mimicry by RKNs suggesting a role for nematode RAR peptides in gall formation.

As the present studies place M. truncatula RARs at the crossroads of root development and responses to nutritional cues, we have renamed CEPs to RARs (Root Architecture Regulators) to more accurately reflect this function, and will be referred to as such from hereon.

The present invention therefore relates to methods for modulating the root architecture of plants, to create plants more suited to their environments, with increased or decreased lateral root development, or with increased nodulation (which in turn should lead to increased nitrogen assimilation).

Nematodes are represented by a wide number of species and constitute a major proportion of the diversity in the entire animal kingdom. A subset of the nematodes is plant parasitic and these have significant negative impacts on plant growth and yield. Of these, root knot nematodes (RKNs) (and not other nematodes) possess and express the RAR family of genes. This was surprising and intriguing. RKNs are obligate parasites infecting a broad spectrum of (probably all) vascular plants, causing crop yield losses estimated at more than USD50 billion per annum. Crop losses of 10-20% are common. Effective RKN control strategies are limited; current strategies are toxic and non-specific as they also kill the much larger and diverse population of benign and ecologically important nematodes in the soil. Central to the RKN-plant interaction is the de novo induction of a root organ (called a knot) in which vascular parenchyma cells develop into multinucleate giant cells that provide a feeding site for the RKN and surrounding cells exhibit various degrees of hypertrophy and hyperplasia. The proliferation of surrounding cells is called a gall. It has long been postulated that factors secreted by RKNs are required to subvert plant development pathways for knot formation. Remarkably, gall formation and symbiotic root nodule formation, a developmental program that provides a reliable N-supply to legumes, share mechanistic features.

During the course of the present studies it has been found that RKNs possess genetic sequences coding for homologues of the RARs, as well as other types of plant growth regulating peptides involved in root development and morphology, including the root growth factors (RGFs), and inflorescence deficient in abscission (IDA) families. This finding supports the view that RKNs subvert plant root development pathways, and that this may be done through secretion of one or more of these plant peptide homologues. Means that limit nematodes from secreting these peptides may enable strategies for at least controlling or limiting RKN damage to plant roots. An RKN-specific virulence target would be welcome, since the vast majority of other soil nematodes are benign to plant growth and serve important ecological functions.

Thus, the present invention not only relates to methods for modulating the root architecture of plants, but also to methods for at least limiting damage to plant roots by RKNs. Such methods may involve: contacting the root zone of a plant with a RAR or a homologue or analogue of a RAR, or a RAR signaling agonist; contacting the root zone of a plant with a RAR and/or a RGF signaling antagonist; introducing one or more mutations or exogenous nucleic acids into one or more plant cells which mutation or introduced exogenous nucleic acid results in modulated RAR or RGF expression by root cells of a plant or by a RKN; introducing one or more mutations or exogenous nucleic acids into one or more plant cells which mutation or introduced exogenous nucleic acid results in modulated RAR or RGF receptor expression by root cells of a plant; introducing one or more mutations or exogenous nucleic acids into one or more plant cells which results in modulated affinity of one or more RARs or RGFs for their respective receptors, which modulated affinity arises through modifications in the RAR(s), RGF(s), their respective receptor(s) or in both expressed RAR(s)/RGF(s) and their respective receptor(s); or introducing one or more exogenous nucleic acids into one or more plant cells which results in expression of one or more root knot nematode RAR or RGF binding agents or antagonists by said cell(s).

The invention also relates to isolated RKN RAR genes, RKN RAR-encoding nucleotide sequences and vectors and transformed cells comprising them, and isolated RKN RARs. The present invention further encompasses isolated RKN RGF-encoding nucleotide sequences and vectors and transformed cells comprising them, and isolated RKN RGFs. Also contemplated by the present invention are RKN IDA-encoding nucleotide sequences and vectors and transformed cells comprising them and isolated RKN IDAs, as well as at least methods for inhibiting RKN damage to plants relating to such materials.

### RARs and Encoding Nucleic Acids and Genes

Herein described and provided are RARs from a wide range of plants, including gymnosperms and angiosperms, as well as root knot nematodes (RKNs), as well as their encoding nucleotides. Previously described Arabidopsis thaliana CEP1 and its encoding nucleotide sequence is excluded from RAR peptides and encoding nucleotides according to the invention per se, but may be used in methods of the invention for modulation of plant root architecture.

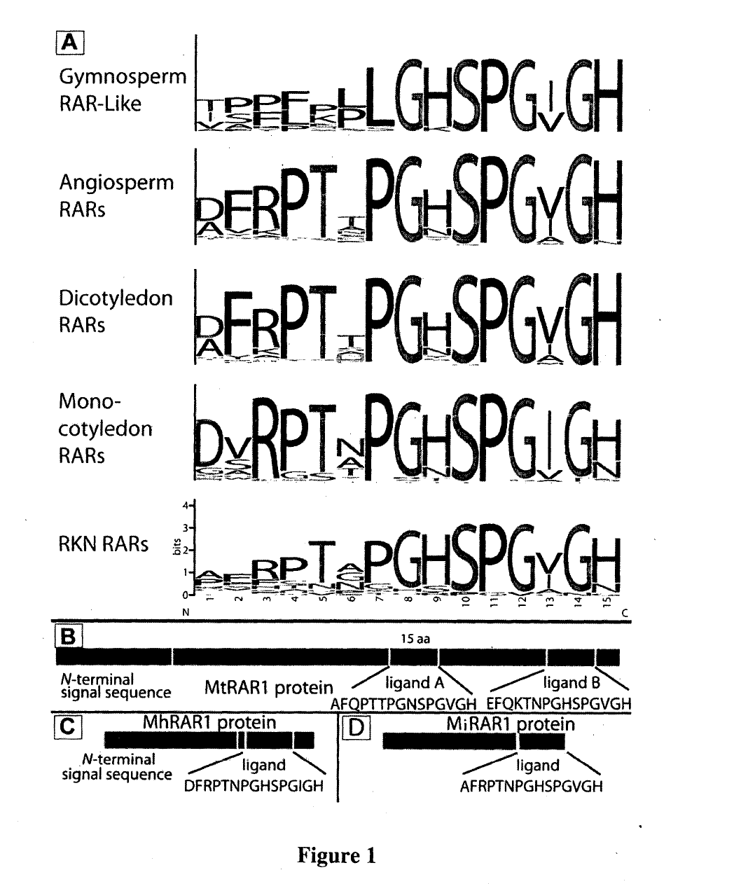

The RARs may broadly have features as shown in FIG. 1A and, according to an embodiment, the RAR may comprise an amino acid sequence selected from SEQ ID NOs: 4, 6, 8, 10, 12, 14, 16, 18, 20, 22, 24, 26, 28, 30, 32, 34, 36 to 147, 338-350, 365, 367, 369, 371, 373, 375, 377, 379, 381, 383, 385, 387-395), or an RAR domain having an amino acid sequence selected from SEQ ID Nos: 148-336, 351-363 or 396-415, or may comprise an amino acid sequence sharing at least 60% identity, at least 70% identity, at least 80% identity, at least 85% identity, at least 90% identity, at least 95% identity, or at least 99% identity with said amino acid sequences.

RAR peptides according to the invention may include modifications to one or more of the amino acids. Such modifications may include natural modifications, such as post-translational modifications, including, for example, phosphorylation, hydroxylation, sulphonation and glycosylation. Such modifications may also be artificially created or instigated. According to an embodiment, an RAR peptide may comprise such modifications may comprise phosphorylation of one or more threonine or serine residues, where present, hydroxylation of one or more proline residues, where present, and sulphonation of the tyrosine at position 2, or at any other position when present, especially when preceded by aspartic acid.

According to another embodiment, the RAR-encoding nucleic acid may comprise a nucleotide sequence selected from SEQ ID NOs 3, 5, 7, 9, 11, 13, 15, 17, 19, 21, 23, 25, 27, 29, 31, 33, 35, 364, 366, 368, 370, 372, 374, 376, 378, 380, 382, 384 or 386, or may comprise a nucleotide sequence sharing at least 60% identity, at least 70% identity, at least 80% identity, at least 85% identity, at least 90% identity, at least 95% identity, or at least 99% identity with a nucleotide sequence selected from SEQ ID NOs 3, 5, 7, 9, 11, 13, 15, 17, 19, 21, 23, 25, 27, 29, 31, 33, 35, 364, 366, 368, 370, 372, 374, 376, 378, 380, 382, 384 or 386.

According to another embodiment, the RAR is a root knot nematode RAR and may have the RKN RAR features shown in FIG. 1A. According to a further embodiment, the RKN RAR may comprise an amino acid sequence selected from SEQ ID NOs 365, 367, 369, 371, 373, 375, 377, 379, 381, 383, 385, 387-395 or comprise an RAR domain having an amino acid sequence selected from SEQ ID Nos: 396-415, or may comprise an amino acid sequence sharing at least 60% identity, at least 70% identity, at least 80% identity, at least 85% identity, at least 90% identity, at least 95% identity, or at least 99% identity with an amino acid sequence selected from SEQ ID. NOs 365, 367, 369, 371, 373, 375, 377, 379, 381, 383, 385, 387-415.

According to another embodiment, the RKN RAR-encoding nucleic acid may comprise a nucleotide sequence selected from SEQ ID NOs 364, 366, 368, 370, 372, 374, 376, 378, 380, 382, 384 or 386, or may comprise a nucleotide sequence sharing at least 60% identity, at least 70% identity, at least 80% identity, at least 85% identity, at least 90% identity, at least 95% identity, or at least 99% identity with a nucleotide sequence selected from SEQ ED NOs 364, 366, 368, 370, 372, 374, 376, 378, 380, 382, 384 or 386.

During the course of the studies leading to the present invention, the whole gene coding for the Medicago truncatula (MtRAR1) was identified, and the promoter found to be regulated by nutrient levels, especially available nitrogen. It is contemplated that such a promoter may be beneficial for expressing RAR peptides during periods of nitrogen limitation. Thus, according to a further embodiment, there is provided a RAR-encoding gene comprising the nucleotide sequence as shown in SEQ ID NO. 101, or comprising a nucleotide sequence sharing at least 60% identity, at least 70% identity, at least 80% identity, at least 85% identity, at least 90% identity, at least 95% identity, or at least 99% identity with the nucleotide sequence as shown in SEQ ID NO. 101. According to a yet further embodiment, there is provided a promoter sequence comprising the nucleotide sequence as shown in SEQ. ID NO. 337, or comprising a nucleotide sequence sharing at least 60% identity, at least 70% identity, at least 80% identity, at least 85% identity, at least 90% identity, at least 95% identity, or at least 99% identity with the nucleotide sequence as shown in SEQ ID NO. 337.

Nucleic acid molecules for identifying other RAR-encoding sequences (and thereby the encoded peptides), or for suppressing the expression of RAR-encoding sequences (plant or RKN) are also contemplated by the present invention. Suitable nucleic acid molecules may be any appropriate sequence which is designed based on any one of the RAR-encoding sequences as disclosed herein (SEQ ID NOs: 3, 5, 7, 9, 11, 13, 15, 17, 19, 21, 23, 25, 27, 29, 31, 33, 35, 364, 366, 368, 370, 372, 374, 376, 378, 380, 382, 384 or 386).

The nucleotide sequence of said nucleic acid molecule may be identical to, or be complementary to at least a portion of any one of SEQ ID NOs: 3, 5, 7, 9, 11, 13, 15, 17, 19, 21, 23, 25, 27, 29, 31, 33, 35, 364, 366, 368, 370, 372, 374, 376, 378, 380, 382, 384 or 386, and may comprise the full sequence, or complement thereof or, may comprise an oligonucleotide from about 10 nucleotides in length to about 100 nucleotides in length, such as from about 10 to about 50 nucleotides in length, about 15 to about 100 nucleotides in length, about 15 to about 50 nucleotides in length, about 10 to about 30 nucleotides in length, or about 15 to about 30 nucleotides in length.

Alternatively, a nucleic acid molecule of the invention may comprise a nucleotide sequence designed based on the amino acid sequence of one of the RARs disclosed herein (SEQ ID NOs: 4, 6, 8, 10, 12, 14, 16, 18, 20, 22, 24, 26, 28, 30, 32, 34, 36 to 147, 338-350, 365, 367, 369, 371, 373, 375, 377, 379, 381, 383, 385, 387-395), or an RAR domain having an amino acid sequence selected from SEQ ID Nos: 148-336, 351-363 or 396-415, using degeneracy of the genetic code, and optionally preferred codon usage information. Suitable nucleic acid molecule sizes are as already discussed immediately above.

Nucleotide sequences according to the invention as described above which may be employed as, or which may be comprised in primers, probes, antisense molecules, microRNA molecules or strands in double-stranded RNAi molecules may comprise one or more modifications as known in the art for stabilising the molecule(s) (for example, against enzymic degradation by ribonucleases), or for increasing the strength of hybridization with complementary molecule(s).

### RGFs and IDAs and Encoding Nucleic Acids and Genes

Also herein described and provided are novel root growth factor (RGF) and putative Inflorescence Deficient for Abscission (IDA) peptide sequences from root knot nematodes, as well as their encoding nucleotide sequences. RGFs and IDAs have been previously described in plants, but not in RKNs. It is contemplated that such RGFs or IDAs, their encoding nucleotide sequences, or both may be used to modulate plant root architecture. It is also contemplated that RKN RGFs or IDAs, or RKN RGF- or IDA-specific binding agents or their encoding nucleotide sequences (or fragments thereof), may be used to control the effects or infestation of plants by RKNs.

The RKN RGF may be any RGF identified from RKNs. According to an embodiment an RKN RGF according to the invention comprises an amino acid sequence as shown in SEQ ID NOs: 416 or 417 or comprise an RGF domain comprising an amino acid sequence as shown in SEQ ID NO: 460 or 461, or may comprise an amino acid sequence sharing at least 60% identity, at least 70% identity, at least 80% identity, at least 85% identity, at least 90% identity, at least 95% identity, or at least 99% identity with said amino acid sequences.

According to another embodiment, the RKN RGF-encoding nucleic acid may comprise a nucleotide sequence designed based on an amino acid sequence as shown in SEQ ID NOs: 416, 417, 460 or 461 using degeneracy of the genetic code, and optionally preferred codon usage information. Suitable nucleic acid molecule sizes are as already discussed immediately above.

The RKN IDAs may be any IDA identified from RKNs. According to an embodiment an RKN IDA according to the invention comprises an amino acid sequence as shown in SEQ ID NO: 418 or may comprise an IDA domain comprising an amino acid sequence as shown in SEQ ED NO: 496, or may comprise an amino acid sequence sharing at least 60% identity, at least 70% identity, at least 80% identity, at least 85% identity, at least 90% identity, at least 95% identity, or at least 99% identity with said amino acid sequences.

Nucleic acid molecules for identifying other RKN RGF- or IDA-encoding sequences (and thereby the encoded peptides), or for suppressing the expression of RKN RGF-encoding sequences are also contemplated by the present invention. Suitable nucleic acid molecules may be any appropriate sequence which is designed based on an amino acid sequence as shown in SEQ ID NOs: 416, 417, 460 or 461 (for RGF-encoding sequences) or SEQ ID NOs: 418 or 496 (for IDA-encoding sequences).

The nucleotide sequence of nucleic acid molecules encoding RGFs or IDAs as described above may comprise the full sequence, or complement thereof or, may comprise an oligonucleotide of from about 10 nucleotides in length to about 100 nucleotides in length, such as from about 10 to about 50 nucleotides in length, about 15 to about 100 nucleotides in length, about 15 to about 50 nucleotides in length, about 10 to about 30 nucleotides in length, or about 15 to about 30 nucleotides in length.

Alternatively, a nucleic acid molecule of the invention may comprise a nucleotide sequence designed based on the amino acid sequence of SEQ ID NOs: 416, 417, 460 or 461 (RGFs) or SEQ ID NOs: 418 or 496 (IDAs) using degeneracy of the genetic code, and optionally preferred codon usage information. Suitable nucleic acid molecule sizes are as already discussed immediately above.

Nucleotide sequences according to the invention as described above which may be employed as; or which may be comprised in, for example, primers, probes, antisense molecules, microRNA molecules, or strands in double-stranded RNAi molecules may comprise one or more modifications as known in the art for stabilising the molecule(s) (for example, against enzymic degradation by ribonucleases), or for increasing the strength of hybridization with complementary molecule(s).

### RAR, RGF or IDA Receptors and Encoding Sequences

Receptors for RARs, RGFs or IDAs and their encoding sequences may be identified, isolated and sequenced by methods well known and understood in the art using RAR, RGF or IDA sequences as disclosed herein. Methods for identifying and characterising plant receptors through knowledge of their ligands are well established and have been described in, for example, Shinya T et at (2010), Plant Cell Physiol 51(2): 262-270, which describes a use of affinity cross-linking with biotinylated ligands to isolate receptors.

### Methods for Modulating Root Architecture

In agriculture it would generally be desirable to be able to create plants which are better suited to the soil environment they are to be grown in, physically or chemically. Such adaptations may include greater nutrient uptake efficiency, a root system having nitrogen-fixing nodules, or a root system having greater extent of nodulation, or a root system with altered horizontal or vertical growth patterns (ie. reduced or greater lateral root development).

One manner of achieving such adaptation(s) may be through increased or decreased expression of RAR or RGF genes (and possibly IDA genes), use of the expressed peptides, binding agents, their receptors and modulation of RAR or RGF signaling.

According to an aspect, methods of the present invention for modulating root architecture may include:

(a) contacting the root zone of said plant with a root architecture regulator peptide (RAR), an analogue thereof or an RAR signaling agonist;

(b) contacting the root zone of said plant with a RAR signaling antagonist;

(c) introducing at least one mutation or exogenous nucleic acid into one or more plant cells which results in modulated RAR expression by root cells of a plant regenerated from or comprising said one or more plant cells or in a root knot nematode;

(d) introducing at least one mutation or exogenous nucleic acid into one or more plant cells which results in modulated RAR receptor expression by root cells of a, plant regenerated from or comprising said one or more plant cells;

(e) introducing at least one mutation or exogenous nucleic acid into one or more, plant cells which results in modulated affinity of one or more RARs for their respective RAR receptors, which modulated affinity arises through modifications in the RAR(s), RAR receptor(s) or in both expressed RAR(s) and RAR receptor(s) expressed by root cells of a plant regenerated from or comprising said one or more plant cells; or

(f) introducing at least one exogenous nucleic acid into one or more plant cells which results in expression of one or more root knot nematode RAR binding agents or antagonists and secretion thereof into the root zone of said plant.

Similar techniques could be applied in the context of RGFs and possibly IDAs.

Of great interest to agriculture is the prospect of increasing nutrient uptake from soils (which may be achieved by increasing or reducing lateral root development, depending on the circumstances), developing nitrogen-fixation in non-legumes, and increased nodulation in legumes.

Methods for decreasing lateral root development by a plant relative to an untreated or wild-type plant, may comprise contacting the root zone of said plant with at least one RAR, an RAR analogue, or an RAR signaling agonist. Methods for decreasing lateral root development by a plant relative to an untreated or wild-type plant, may also comprise treating seeds of plants with at least one RAR, an RAR analogue, or an RAR signaling agonist prior to sowing.

RARs for use in such methods may be any RAR as mentioned above. According to an embodiment, the RAR is not an Arabidopsis thaliana CEP1 peptide. According to another embodiment, the RAR is an RAR comprising an amino acid sequence as shown in SEQ ID NOs: 4, 6, 8, 10, 12, 14, 16, 18, 20, 22, 24, 26, 28, 30, 32, 34, 36 to 147, 338-350, 365, 367, 369, 371, 373, 375, 377, 379, 381, 383, 385, 387-395 or comprises an RAR domain having an amino acid sequence selected from SEQ ID Nos: 148-336, 351-363 or 396-415. According to another embodiment, the RAR is an RKN RAR and, according to a further embodiment, comprises an amino acid sequence as shown in SEQ ID NOs 365, 367, 369, 371, 373, 375, 377, 379, 381, 383, 385, 387-395 or comprises an RAR domain having an amino acid sequence selected from SEQ ID Nos: 396-415 or is encoded by a nucleic acid comprising a nucleotide sequence selected from SEQ ID NOs 364, 366, 368, 370, 372, 374, 376, 378, 380, 382, 384 or 386.

An RAR analogue for use in a method of the present invention may be any artificial or natural substance that resembles the protein in function. For example, an RAR analogue may bind an RAR receptor and thereby bring about the same or similar result as if a natural RAR had bound to the receptor. In an embodiment such analogues may also resemble the protein in structure. Analogues contemplated in an embodiment of the present invention include fully or partially peptidomimetic compounds based on the structures of the RARs disclosed herein. Peptidomimetic compounds (compounds designed to mimic biologically active peptides, but comprising structural differences—to provide advantages, especially in terms of stability, but also interaction with ligands/binding partners or substrates—and comprising unnatural amino acids or other unusual compounds) and their design is well-studied and is described in, for example, Floris M. et al (2011), Nucleic Acids Research 39(18): W261-269. Alternatively, an RAR analogue may be a peptide that resembles an RAR in function and activity, but comprise one or more amino acid substitutions, deletions or insertions compared to the subject RAR, and may share at least about 50% amino acid identity, at least about 60% identity, at least about 70% identity, at least about 80% identity, at least about 90% identity, at least about 95% identity, or at least about 99% identity with the amino acid sequence of the subject RAR.

An RAR signaling agonist for use in a method of the present invention may be any molecule that binds with a RAR receptor to trigger a physiological response Usually triggered by a RAR peptide when it binds to the receptor. For example, a RAR agonist may be a molecule that binds to a RAR receptor to trigger a root architectural response.

Methods for decreasing lateral root development by a plant relative to an untreated or wild-type plant, may also comprise introducing into one or more plant cells at least one exogenous RAR-encoding nucleic acid into one or more plant cells. Plants with increased RAR expression by root cells of the plants may be regenerated from, or comprise such transformed plant cells.

Transgenic plants with an introduced RAR-encoding sequence may be generated using standard plant transformation methods known to those skilled in the art including, for example, Agrobacterium-mediated transformation, cation or polyethylene glycol treatment of protoplasts, calcium phosphate precipitation, electroporation, microinjection, viral infection, protoplast fusion, microparticle bombardment, agitation of cell suspensions in solution with microbeads or microparticles coated with the transforming DNA, direct DNA uptake, liposome-mediated DNA uptake, and the like, as also described in a wide range of publicly available texts, such as: “Methods for Plant Molecular Biology” (Weissbach & Weissbach, eds., 1988); Clough, S. J. and Bent, A. F. (1998) “Floral dip: a simplified method for Agrobacterium-mediated transformation of Arabidopsis thaliana” Plant J. 16, 735-743; “Methods in Plant Molecular Biology” (Schuler & Zielinski, eds., 1989); “Plant Molecular Biology Manual” (Gelvin, Schilperoort, Verma, eds., 1993); and “Methods in Plant Molecular Biology-A Laboratory Manual” (Maliga, Klessig, Cashmore, Gruissem & Varner, eds., 1994). See also Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000), these references being incorporated herein by cross-reference.

The coding region may also be operably linked to an appropriate 3′ regulatory sequence. For example, the nopaline synthetase (NOS) polyadenylation region or the octopine synthetase (OCS) polyadenylation region may be used.

The preferred method of transformation may depend upon the plant to be transformed. Agrobacterium vectors are often used to transform dicot species. For transformation of monocot species, biolistic bombardment with particles coated with transforming DNA and silicon fibers coated with transforming DNA are often useful for nuclear transformation. However, Agrobacterium-mediated transformation of monocotyledonous species, including wheat, are now known (see, for example, International patent publications WO 97/48814; see also Hiei, Y. et al (1994), Plant J. 6(2):271-282 and international patent publication WO 92/06205).

An RAR-encoding sequence can be comprised in a vector. Representative vectors include plasmids, cosmids, and viral vectors. Vectors can also comprise nucleic acids including expression control elements, such as transcription/translation control signals, origins of replication, polyadenylation signals, internal ribosome entry sites, promoters, enhancers, etc., wherein the control elements are operatively associated with a nucleic acid encoding a gene product. Selection of these and other common vector elements are conventional and many such sequences can be derived from commercially available vectors. See, for example, Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000).

According to an embodiment, the vector is an expression vector capable of directing the transcription of an RAR-encoding sequence into RNA.

DNA constructs for transforming a selected plant may comprise a coding sequence of interest operably linked to appropriate 5′ regulatory sequences (e.g., promoters and translational regulatory sequences) and 3′ regulatory sequences (e.g., terminators). In a preferred embodiment, the coding region is placed under a powerful constitutive promoter, such as the Cauliflower Mosaic Virus (CaMV) 35S promoter or the figwort mosaic virus 35S promoter. Other constitutive promoters contemplated for use in the present invention include, but are not limited to: T-DNA mannopine synthetase, nopaline synthase (NOS) and octopine synthase (OCS) promoters.

Using an Agrobacterium binary vector system for transformation, the selected coding region, under control of a constitutive or inducible promoter as described above, may be linked to a nuclear drug resistance marker, such as kanamycin resistance. Other useful selectable marker systems include, but are not limited to: other genes that confer antibiotic resistances (e.g., resistance to hygromycin or bialaphos) or herbicide resistance (e.g., resistance to sulfonylurea, phosphinothricin, or glyphosate).

According to an embodiment, the RAR-encoding sequence is operably linked to a promoter which is constitutive or inducible. An inducible promoter, for the purposes of the present invention, may be inducible by any appropriate stimulus. According to certain embodiments, an inducible promoter for use according to the present invention may be inducible by nutrient, drought, or other abiotic stress. According to an embodiment, an inducible promoter for use according to the present invention is inducible by nutrient status, such as by nitrogen starvation or by high carbon dioxide.

According to another embodiment, the RAR-encoding sequence is operably linked to a promoter which is root-specific.

According to another embodiment, the RAR-encoding sequence comprises a secretion signal sequence.

According to an embodiment, the RAR-encoding sequence is operably linked to a promoter comprising the nucleotide sequence as shown in SEQ ID NO: 337 or a homologue thereof sharing at least 60% identity with SEQ ID NO: 337. Alternatively, the promoter may be a root-specific glutamine synthetase gene promoter.

According to an embodiment, a method of the invention for modulating the root architecture of a plant, relative to a wild-type plant, comprises introducing into one or more plant cells the Medicago truncatula RAR1 gene, including the promoter sequence, disclosed herein as SEQ ID NO: 337, and the RAR-encoding sequence, disclosed herein as SEQ ID NO: 15.

The coding region may also be operably linked to an appropriate 3′ regulatory sequence. For example, the nopaline synthetase (NOS) polyadenylation region or the octopine synthetase (OCS) polyadenylation region may be used.

Using an Agrobacterium binary vector system for transformation, the selected coding region, under control of a constitutive or inducible promoter as described above, may be linked to a nuclear drug resistance marker, such as kanamycin resistance. Other useful selectable marker systems include, but are not limited to: other genes that confer antibiotic resistances (e.g., resistance to hygromycin or bialaphos) or herbicide resistance (e.g., resistance to sulfonylurea, phosphinothricin, or glyphosate).

Any of the methods of the present invention, as discussed above or below, can be used to transform any plant cell. In this manner, genetically modified plants, plant cells, plant tissue, seed, and the like can be obtained. The plant cell(s) to be transformed may be a plant cell from any plant selected from angiosperms or gymnosperms. Non-exhaustive examples of angiosperms for treatment or transformation by a method of the invention may include any member of the Aceraceae, Anacardiaceae, Apiaceae, Asteraceae, Betulaceae, Brassicaceae, Buxaceae, Chenopodiaceae/Amaranthaceae, Compositae, Cucurbitaceae, Fabaceae, Fagaceae, Gramineae, Juglandaceae, Lamiaceae, Lauraceae, Leguminosae, Moraceae, Myrtaceae, Oleaceae, Platanaceae, Poaceae, Polygonaceae, Rosaceae, Rutaceae, Salicaceae, Solanaceae, Ulmaceae or Vitaceae. Examples of gymnosperms for treatment or transformation by a method of the invention may include any member of the Cuppressaceae, Pinaceae, Taxaceae or Taxodiaceae.

Cells which have been transformed may be grown into plants in accordance with conventional methods as are known in the art (See, for example, McCormick, S. et al (1986), Plant Cell Reports 5:81-84). The resulting plants may be self-pollinated, pollinated with the same transformed strain or different strains or hybridised, and the resulting plant(s) having modulated root architecture compared to wild-type plants identified. Two or more generations may be grown to ensure that this phenotypic characteristic is stably maintained. Alternatively, in vegetatively propagated crops, mature mutant/transgenic plants may be propagated by cutting or by tissue culture techniques to produce identical plants. Selection of mutant/transgenic plants can be carried out and new varieties may be obtained and propagated vegetatively for commercial use. For a general description of plant transformation and regeneration see, for example, Walbot et al. (1983) in “Genetic Engineering of Plants”, Kosuge et al. (eds.) Plenum Publishing Corporation, 1983 and “Plant Cell, Tissue and Organ Culture: Fundamental. Methods”, Gamborg and Phillips (Eds.), Springer-Verlag, Berlin (1995). See also Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000).

Plants transformed/mutated by the methods of the invention may be screened based on expression of a marker gene, by detecting root architecture modulation by the introduced nucleotide sequence, molecular analysis using specific oligonucleotide probes and/or amplification of the target gene.

Modulation of root architecture may also be achieved through increasing or decreasing RAR signaling by modulation of the affinity of one or more RARs for the corresponding RAR receptor(s) through mutation of the RAR(s) or RAR receptor(s), or introducing exogenous sequences coding for one or more RAR(s) or RAR receptor(s) with desirable signaling interaction attributes. Methods for introducing mutations into target nucleotide sequences, and screening thereof, are described further below.

Furthermore, it is also known that microRNAs (small post-transcriptional regulators that bind to complementary sequences on target mRNAs, resulting in translational repression or target degradation and gene silencing) are expressed by plants, and that these play a significant role in control of most, if not all, plant development regulatory mechanisms. See, for example, Voinnet O (2009) Cell 136(4): 669-687; Jones-Rhoades M W et al (2006) Annual Review of Plant Biology 57:19-53. It is therefore contemplated that RARs and RAR receptor(s) would be subject to such regulation, the amount of mRNA encoding these species present in plant cells being regulated by expression of such microRNAs. Control or inhibition of expression of such microRNAs, or control of their interaction with targeted RAR or RAR receptor mRNAs or their inactivation (such as by use of microRNA decoys—see, for example, Ivashuta S et al (2011) PLoS ONE 6(6): e21330) is therefore contemplated as a further, or as a complementary means for modulating plant root architecture. Identification of endogenous plant microRNAs which target RAR-encoding or RAR receptor-encoding mRNAs may be achieved using the nucleotide sequence encoding the subject RAR or RAR receptor, or homologues thereof by methods well known in the art. Alternatively an artificial microRNA approach could be adopted as disclosed in Schwab R et al. (2006), “Highly Specific Gene Silencing by Artificial MicroRNAs in Arabidopsis”, Plant Cell 18:1121-1133, hereby incorporated in its entirety by cross-reference. RNAi gene silencing is another approach to silence plant or nematode genes in planta, as disclosed in Rosso M N et al. (2009) “RNAi and Functional Genomics in Plant Parasitic Nematodes” Annual Review of Phytopathology 47: 207-232, and Plant Biotechnol J. (2011) 10:1467-7652. “Biotechnological application of functional genomics towards plant-parasitic nematode control”.

Alternatively, avoidance of microRNA suppression of RAR or RAR receptor expression may be achieved by introducing into a subject plant, as described above, an exogenous RAR-encoding or RAR receptor-encoding sequence sufficiently different to any endogenous homologue sequences such that the microRNA is insufficiently homologous to the introduced sequence to achieve suppression. RKN RAR-encoding sequences may be advantageous in this regard.

The methods described above, may result in plants having greater primary root development, at the expense of lateral root development, and may exhibit greater density of root hairs at periodic root bumps. The methods may also result in an increase in symbiotic nitrogen-fixing nodule numbers on the root(s) of plants, such as members of the Fabaceae or Leguminoseae, or even nitrogen-fixing nodules on the roots of plants which normally do not have nitrogen-fixing nodules (such as, for example, cereals).

Methods of the present invention for modulating root architecture, based on the herein disclosed understanding of RARs and their effects, may also include methods for promoting lateral root development in plants compared to wild-type plants. The studies leading to the present invention found that overexpression of RARs leads to suppression of lateral root development. It is contemplated that suppression of RAR expression will, conversely, promote lateral root growth and development.

Methods of the invention for promoting lateral root growth and development, relative to an untreated or wild-type plant, may comprise contacting the root zone of said plant with a RAR antagonist or introducing at least one mutation or at least one exogenous nucleic acid into one or more plant cells which at least one mutation or nucleic acid results in:
(i) decreased expression of one or more RARs, decreased expression of one or more RAR receptors, or decreased expression of one or more RARs and one or more RAR receptors by root cells of a plant regenerated from or comprising said one or more plant cells, wherein said decreased expression of said RAR(s) or RAR receptor(s) occurs under conditions which would otherwise promote expression of said RAR(s) or RAR receptor(s); or
(ii) reduced affinity of one or more RARs for their respective RAR receptors, which reduced affinity arises through modifications in the RAR(s), RAR receptor(s) or in both expressed RAR(s) and RAR receptor(s) expressed by root cells of a plant regenerated from or comprising said one or more plant cells.

A RAR signaling antagonist for use in a method of the present invention may be any substance that interferes with the physiological response usually triggered by an RAR when it binds to its receptor, or which interferes with binding of the RAR to its receptor. For example, a RAR antagonist may be a substance that binds to a RAR or a RAR receptor to inhibit interaction between the RAR and the RAR receptor, which interaction would trigger a root architectural response. RAR antagonists may include antibodies to RARs or RAR receptors.

Decreased expression of one or more RARs, one or more RAR receptors, or both, may be achieved by any suitable technique, many being known in the art, including, antisense technology, interfering RNA technology, ribozyme technology, mutation of the gene(s) to create null mutants, and replacement or mutation of regulatory regions to reduce or obviate gene expression.

For example, a method of the invention may comprise inserting into said one or more plant cells exogenous nucleic acid which inhibits expression of the activity of an endogenous RAR (for example, via regulatory regions controlling expression of a RAR, via the RAR-encoding sequence, or via mRNA translated from the RAR-encoding sequence), or which replaces expression of an endogenous RAR or homologue thereof with expression of an exogenous protein. The exogenous protein may be an exogenous mutant RAR or homologue thereof, or any other suitable protein, such as a protein providing a screenable phenotype.

According to an embodiment for carrying out a method of the invention, a plant with promoted lateral root growth or development, relative to a wild-type plant, may be created by inhibiting translation of an RAR mRNA by RNA interference (RNAi), antisense or post-transcriptional gene silencing techniques. The RAR gene targeted for down-regulation, or a fragment thereof, may be utilized to control the production of the encoded protein. Full-length antisense molecules can be used for this purpose. Alternatively, double stranded oligonucleotides, sense and/or antisense oligonucleotides, or a combination thereof targeted to specific regions of the RAR-encoded RNA may be utilized. The use of oligonucleotide molecules to decrease expression levels of a pre-determined gene is known in the art (see, for example, Hamilton, A. J. and Baulcombe, D. C. (1999), Science 286:950-952; Waterhouse P. M. et al (1998), Proc. Natl. Acad. Sci. USA 95:13959-13964; Fire et al. (1998) Nature 391: 806-811; Hammond, et al. (2001) Nature Rev, Genet. 2: 110-1119; Hammond et al. (2000) Nature 404: 293-296; Bernstein et al. (2001) Nature 409: 363-366; Elbashir et al (2001) Nature 411: 494-498; and International patent publications WO 99/53050, WO 99/49029, WO 99/32619, the disclosures of which are incorporated herein by reference). RNA interference (RNAi) refers to a means of selective post-transcriptional gene silencing by destruction of specific mRNA by small interfering RNA molecules (siRNA). The siRNA is typically generated in vivo by cleavage of double stranded RNA, where one strand is identical to the message to be inactivated. Double-stranded RNA molecules may be synthesised in which one strand is identical to a specific region of the mRNA transcript and introduced directly. Alternatively corresponding dsDNA can be employed, which, once presented intracellularly is converted into dsRNA. Methods for the synthesis of suitable single or double-stranded oligonucleotides, or constructs capable of expressing them in planta for use in antisense or RNAi and for achieving suppression of gene expression are known to those of skill in the art. The skilled addressee will appreciate that a range of suitable single- or double-stranded oligonucleotides capable of inhibiting the expression of the disclosed polynucleotides, or constructs capable of expressing them in planta can be identified and generated based on knowledge of the sequence of the gene in question using routine procedures known to those skilled in the art without undue experimentation. Oligonucleotide molecules may be provided in situ by transforming plant cells with a DNA construct which, upon transcription, produces double stranded and/or antisense RNA sequences, which may be full-length or partial sequences. The gene silencing effect may be enhanced by over-producing both sense and/or antisense sequences (which may be full-length or partial) so that a high amount of dsRNA is produced.

Suitable molecules can be manufactured by chemical synthesis, recombinant DNA procedures or by transcription in vitro or in vivo when linked to a promoter, by methods known to those skilled in the art, and may be modified by chemistries well known in the art for stabilising the molecules in vivo and/or enhancing or stabilising their interaction with target complexes or molecules.

Those skilled in the art will appreciate that there need not necessarily be 100% nucleotide sequence match between the target sequence and the RNAi sequence. The capacity for mismatch is dependent largely on the location of the mismatch within the sequences. In some instances, mismatches of 2 or 3 nucleotides may be acceptable but in other instances a single nucleotide mismatch is enough to negate the effectiveness of the siRNA. The suitability of a particular siRNA molecule may be determined using routine procedures known to those skilled in the art without undue experimentation.

Sequences of/for antisense constructs may be derived from various regions of the target gene(s). Antisense constructs may be designed to target and bind to regulatory regions of the nucleotide sequence, such as the promoter, or to coding (exon) or non-coding (intron) sequences. Antisense constructs of the invention may be generated which are at least substantially complementary across their length to the region of the gene in question. Binding of an antisense construct to its complementary cellular sequence may interfere with transcription, RNA processing, transport, translation and/or mRNA stability.

In particular embodiments of the invention, suitable sequences encoding inhibitory nucleic acid molecules may be administered in a vector. The vector may be a plasmid vector, a viral vector, or any other suitable vehicle adapted for the insertion of foreign sequences and introduction into eukaryotic cells. Preferably the vector is an expression vector capable of directing the transcription of the DNA sequence of an inhibitory nucleic acid Molecule of the invention into RNA.

Transgenic plants expressing a sense and/or antisense RAR-encoding sequence, or a portion thereof under an inducible promoter are also contemplated to be within the scope of the present invention. Promoters inducible by nutrient conditions, such as low nitrogen are especially contemplated by the present invention. Promoters which may be used according to the invention may include, for example, the root-specific glutamine synthetase gene promoters, or promoters such as the M. truncatula promoter disclosed herein as SEQ ID NO: 337, for expression in the transformed plant.

Suitable constructs and vectors and transformation techniques for introducing inhibitory nucleic acids or constructs encoding them into plants, as well as methods for regenerating plants from transformed cells have already been discussed above.

As mentioned above, a further means of inhibiting gene expression may be achieved by introducing catalytic antisense nucleic acid constructs, such as ribozymes, which are capable of cleaving RNA transcripts and thereby preventing the production of the native protein. Ribozymes are targeted to and anneal with a particular sequence by virtue of two regions of sequence complementarity to the target flanking the ribozyme catalytic site. After binding, the ribozyme cleaves the target in a site-specific manner. The design and testing of ribozymes which specifically recognize and cleave sequences of interest, such as RAR-encoding sequences and RAR receptor-encoding sequences, can be achieved by techniques well known to those in the art (for example Lieber and Strauss, (1995) Mol. Cell. Biol. 15:540-551, and de Feyter R and Gaudron J (1998) “Expressing Ribozymes in Plants”, Methods in Molecular Biology 74: 403-415, the disclosures of which are incorporated herein by reference).

Suitable constructs and vectors and transformation techniques for introducing ribozymes or constructs encoding them into plants, as well as methods for regenerating plants from transformed cells have already been discussed above.

Similar to the situation described above, where RAR or RAR receptor expression or overexpression is promoted to modulate root architecture, microRNA manipulation may also be employed to suppress RAR or RAR receptor expression. It is contemplated that overexpression, or constitutive expression of microRNAs specifically targeting subject RAR-encoding or RAR receptor-encoding sequences may be employed to suppress expression of those sequences to promote lateral root growth or development. Alternatively, exogenous nucleotide construct(s) encoding microRNAs specific for subject RAR-encoding or RAR receptor-encoding sequences, under the control of desired regulatory sequences may be introduced into plant cells, as disclosed in Schwab R et al. (2006), “Highly Specific Gene Silencing by Artificial MicroRNAs in Arabidopsis”, Plant Cell 18:1121-1133. Modulation of root architecture by a method of the present invention may also be achieved by modulating the affinity of RAR(s) for respective RAR receptor(s). Reduced affinity or reduced expression of one or more RARs for their respective RAR receptors, or vice versa, so as to promote lateral root growth in plants, rather than primary root growth, may be effected by a number of means, such as through modifications in endogenous sequences coding for the RAR(s) or RAR receptor(s), or by replacing the RAR-encoding sequence(s) or RAR receptor-encoding sequence(s) with sequences coding for an RAR or RAR receptor having less binding affinity for the corresponding molecule.

Modifications in endogenous sequences coding for RAR(s) or RAR receptor(s) may be achieved by in situ mutation, either by physical or chemical mutagenesis or by introduction of exogenous nucleic acid which introduces mutations into the target nucleotide sequence(s).

In one embodiment the exogenous nucleic acid may comprise an oligonucleotide or polynucleotide which introduces a mutation comprising single or multiple nucleotide insertions, deletions or substitutions into the endogenous nucleotide sequence encoding an RAR or an RAR receptor, or a homologue(s) thereof via homologous recombination.

Single or multiple nucleotide insertions, deletions or substitutions may be introduced via recombination of the target mutation site with an introduced targeting nucleotide sequence. Such an introduced nucleotide sequence may, for example, comprise a nucleotide sequence to be introduced into the genome flanked either side by nucleotide sequences homologous to target sequences contiguous in or located either side of a desired mutation insertion point. In accordance with the methods of the present invention, a nucleotide sequence to be introduced into the genome may also include a selectable marker operably linked to desired regulatory regions (which may include, for example, a root-specific promoter).

The nucleotide sequences homologous to the target sequences may be isogenic with the target sequences to thereby promote the frequency of homologous recombination.

Homologous nucleotide sequences that are not strictly isogenic to the target sequences can also be used. Although mismatches between the homologous nucleotide sequences and the target sequences can adversely affect the frequency of homologous recombination, isogenicity is not strictly required and substantial homology may be sufficient. For the purposes of the present invention, the level of homology between the homologous sequences and the target sequences may be at least about 90% identity, at least about 95% identity, at least about 99% identity or 100% identity.

A targeting nucleotide sequence can be comprised in a vector. Representative vectors include plasmids, cosmids, and viral vectors. Vectors can also comprise nucleic acids including expression control elements, such as transcription/translation control signals, origins of replication, polyadenylation signals, internal ribosome entry sites, promoters, enhancers, etc., wherein the control elements are operatively associated with a nucleic acid encoding a gene product. Selection of these and other common vector elements are conventional and many such sequences can be derived from commercially available vectors. See, for example, Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000).

A targeting vector can be introduced into targeting cells using any suitable method known in the art for introducing DNA into cells, including but not limited to microinjection, electroporation, calcium phosphate precipitation, liposome-mediated delivery, viral infection, protoplast fusion, and particle-mediated uptake.

Optionally, a targeting DNA is co-administered with a recombinase, for example recA, to a target cell to thereby enhance the rate of homologous recombination. The target cell(s) may already comprise, or have been transformed to comprise suitable recombinase target sequences, if required. For example, a recombinase protein(s) can be loaded onto a targeting DNA as described in U.S. Pat. No. 6,255,113. To enhance the loading process, a targeting DNA can contain one or more recombinogenic nucleation sequences. A targeting DNA can also be coated with a recombinase protein by pre-incubating the targeting polynucleotide with a recombinase, whereby the recombinase is non-covalently bound to the polynucleotide. See, for example, A. Vergunst et al (1998), Nucleic Acids Res. 26:2729 and A. Vergunst and P. Hooykaas (1998), Plant Molec. Biol. 38:393 406, International patent publications WO 99/25821, WO 99/25840, WO 99/25855, and WO 99/25854 and U.S. Pat. Nos. 5,780,296, 6,255,113, and 6,686,515.

Suitable constructs and vectors and transformation techniques for introducing targeting sequences as discussed above, as well as methods for regenerating plants from transformed cells have already been discussed above.

Plants transformed/mutated by the methods of the invention may be screened based on the lack of or reduced expression, or of overexpression of a RAR or RAR receptor protein, or homologues thereof, or of their activity or by observation of modulated root growth compared to wild-type plants, molecular analysis using specific oligonucleotide probes and/or amplification of the target gene.

A mutation which results in reduced expression of RAR(s) or RAR receptor(s), or homologues thereof in plant cells may be introduced into the one or more plant cells by any appropriate methods as are known in the art. For example, suitable methods may comprise exposing the one or more plant cells (which may be plant seed cells, or cells of a part of a plant, as well as isolated plant cells) to chemical or physical mutagenic means, or insertional mutagenic means such as transposons, retrotransposons, retroviruses, or T-DNA. Suitable materials and methods for introducing mutations into a plant genome are also described in, for example, International patent publication WO 98/26082, “Arabidopsis Protocols” (2^{nd} Edition, Salinas, J. and Sanchez-Serrano, J., eds, Methods in Molecular Biology 323 (2006), Humana Press), and “Current Protocols in Molecular Biology” (Ausubel et al. (eds), John. Wiley & Sons (2000)), herein incorporated by reference.

The mutation may also be introduced into the one or more plant cells by crossing a wild-type plant with a plant comprising a desirable mutation (as determined previously by genetic screening and/or analysis—plants comprising a desired mutation may already exist in available plant germplasm/culture/seed collections/varieties), and plants may be generated from the resulting seed and then screened for inheritance of the mutation.

The mutation(s) may be introduced into one or more sequence(s) encoding RAR(s) or RAR receptor(s), or may be introduced into other sequences affecting expression of those proteins (such as upstream sequences, including promoters).

According to an embodiment of the invention, a mutation is introduced into a nucleotide sequence encoding a RAR or RAR receptor or a homologue thereof in one or more plant cells, and may comprise an insertion, deletion or substitution of one or more nucleotides in the nucleotide sequence encoding the RAR or RAR receptor or homologue thereof. In one embodiment the mutation is a RAR or RAR receptor null mutation. Alternatively, the mutation may result in an expressed product which, however, has at least reduced affinity for its binding partner.

The methods of the present invention can employ any mutagenic agent known in the art (employing methods also known in the art) including, but not limited to ultraviolet light, X-ray radiation, gamma radiation or fast neutron mutagenesis, N-ethyl-N-nitrosourea (ENU), methylnitrosourea (MNU), procarbazine (PRC), triethylene melamine (TEM), acrylamide monomer (AA), chlorambucil (CHL), melphalan (MLP), cyclophosphamide (CPP), diethyl sulfate (DES), ethyl methane sulfonate (EMS), methyl methane sulfonate (MMS), 6-mercaptopurine (6-MP), mitomycin-C(MMC), N-methyl-N′-nitro-N-nitrosoguanidine (MNNG), ³H₂O, and urethane (UR).

The frequency of genetic modification upon exposure to one or more mutagenic agents can be modulated by varying dose and/or repetition of treatment, and can be tailored for a particular application. In one embodiment, the treatment dose and regimen does not induce substantial cytotoxicity to the one or more cells.

Mutations in RAR(s) or RAR receptor(s) or homologues thereof can be detected and followed (through generations) by probing with known RAR-encoding sequence(s), such as are disclosed herein (see SEQ ID NOs: 3, 5, 7, 9, 11, 13, 15, 17, 19, 21, 23, 25, 27, 29, 31, 33, 35, 364, 366, 368, 370, 372, 374, 376, 378, 380, 382, 384 or 386) or RAR receptor-encoding sequence(s), which may be identified as described above, using techniques well known in the art and suitable probes or primers based on the gene(s) or nucleotide sequence(s) encoding RAR(s), RAR receptor(s) or homologues thereof.

If the mutation is in a sequence other than RAR-encoding sequence(s) or RAR receptor-encoding sequence(s), the mutation may need to be identified, located and/or characterised before it can be traced/followed through plant generations. Suitable methods for identifying, locating and characterising unknown mutations are known to those in the art and are described in a number of well-known standard texts, such as Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000). See also Rossel, J. B., Cuttriss, A. and Pogson, B. J. “Identifying Photoprotection Mutants in Arabidopsis thaliana” in Methods in Molecular Biology 274: 287-299 (Carpentier, R. ed, Humana Press). More recent methods for identifying mutant alleles include ‘Tilling’ and high resolution melts (HRMs).

TILLING (Targeting Induced Local Lesions in Genomes) is a method in molecular biology that allows directed identification of mutations in a specific gene. The method combines a standard technique (for example, mutagenesis with a chemical mutagen such as Ethyl methanesulfonate (EMS)) with a sensitive DNA screening-technique that identifies single base mutations (also called point mutations) in a target gene. The first paper describing TILLING in Arabidopsis (McCallum C M, Comai L, Greene E A, Henikoff S, “Targeted screening for induced mutations”, Nat Biotechnol. (2000) April; 18(4):455-7, hereby incorporated by cross-reference) used dHPLC HPLC to identify mutations. The method was made more high throughput by using the restriction enzyme Cel-1 combined with a gel based system to identify mutations (Colbert T, Till B J, Tompa R, Reynolds S, Steine M N, Yeung A T, McCallum C M, Comai L, Henikoff S, “High-throughput screening for induced point mutations”, Plant Physiol. (2001) June; 126(2):480-4, also hereby incorporated by cross-reference). Other methods of mutation detection, such as resequencing DNA, have been combined for TILLING. TILLING has since been used as a reverse genetics method in other organisms such as zebrafish, corn, wheat, rice, soybean, tomato and lettuce. See also: McCallum C M, Comai L, Greene E A, Henikoff S. “Targeting induced local lesions in genomes (TILLING) for plant functional genomics” Plant Physiol. (2000) June; 123(2):439-42; Colbert T, Till B J, Tompa R, Reynolds S, Steine M N, Yeung A T, McCallum C M, Comai L, Henikoff S. High-throughput screening for induced point mutations”, Plant Physiol. (2001) June; 126(2):480-4; Draper B W, McCallum C M, Stout J L, Slade A J, Moens C B, “A high-throughput method for identifying N-ethyl-N-nitrosourea (ENU)-induced point mutations in zebrafish”, Methods Cell Biol. (2004); 77:91-112; and Slade A J, Fuerstenherg S I, Loeffler D, Steine M N, Facciotti D, “A reverse genetic, nontransgenic approach to wheat crop improvement by TILLING”, Nat Biotechnol. (2005) January; 23(1):75-81, also hereby incorporated by cross-reference.

HRM (High Resolution Melt) is a recent development that can greatly extend the utility of traditional DNA melting analysis by taking advantage of recent improvements in high resolution melt instrumentation and the development of double strand specific DNA (dsDNA) binding dyes that can be used at high enough concentrations to saturate all double stranded sites produced during PCR amplifications (see http://www.corbettlifescience.com/control.cfm?page=Introduction_{—}4 &bhcp=1), as well as: Dufresne S D, Belloni D R, Wells W A, Tsongalis G J, “BRCA1 and BRCA2 Mutation. Screening using SmartCyclerII high-resolution melt curve analysis”, Arch Pathol Lab Med (2006) 130: 185-187; Graham R, Liew M, Meadows C, Lyon E, Wittwer C T, “Distinguishing different DNA heterozygotes by high resolution melting”, Clinical Chemistry (2005) 51: 1295-1298; Hermann M G, Durtschl J D, Bromley K, Wittwer C T, Voelkerding K V, “Amplicon DNA melting analysis for mutation scanning and genotyping: cross-platform comparison of instruments and dyes”, Clinical Chemistry (2006) 52: 494-503; Liew M, Pryor R, Palais R, Meadows C, Erali M, Lyon E, Wittwer C, “Genotyping of single nucleotide polymorphisms by high resolution melting of small amplicons”, Clinical Chemistry (2004) 50: 1156-1164; Margraf R L, Mao R, Highsmith W E, Holtegaard L M, Wittwer C T, “Mutation Scanning of the RET protooncogene using high resolution melting analysis”, Clinical Chemistry (2006) 52: 138-141; NGRL (Wessex) Reference Reagent Report January 2006, “Plasmid based generic mutation detection reference reagents; production and performance indicator field trial” (www.ngrl.org.uk/Wessex/downloads.htm); NGRL (Wessex) Reference Reagent Report January 2006. “Production and field trial evaluation of reference reagents for mutation screening of BRCA1, BRCA2, hMLH1 and MHS2” (www.ngrl.org.uk/Wessex/downloads.htm); NGRL (Wessex) Reference Reagent Report June 2006, “Mutation Scanning by High Resolution Melts: Evaluation of Rotor-Gene™ 6000 (Corbett Life Science), HR-1™ and 384 well LightScanner™ (Idaho Technology)” (www.ngrl.org.uk/Wessex/downloads.htm); Reed G H, Wittwer C T, “Sensitivity and specificity of single-nucleotide polymorphism scanning by high resolution melting analysis”, Clinical Chemistry (2004) 50: 1748-1754; Willmore-Payne C, Holden J A, Tripp S, Layfield U, “Human malignant melanoma: detection of BRAF- and c-kit-activating mutations by high-resolution amplicon melting analysis”, Human Pathology (2005) 36: 486-493; Wittwer C T, Reed G H, Gundry C N, Vandersteen J G, Pryor R J, “High-resolution genotyping by amplicon melting analysis using LCGreen” Clinical Chemistry (2003) 49: 853-860; Worm J, Aggerholm A, Guldberg P, “In-tube DNA methylation profiling by fluorescence melting curve analysis” Clinical Chemistry (2001) 47: 1183-1189; Zhou L, Myers A N, Vandersteen J G, Wang L, Wittwer C T, “Closed-tube genotyping with unlabeled oligonucleotide probes and a saturating DNA dye”, Clinical Chemistry (2004) 50: 1328-1335; and Zhou L, Wang L, Palais R, Pryor R, Wittwer C T, “High-resolution DNA melting analysis for simultaneous mutation scanning and genotyping in solution”, Clinical Chemistry (2005) 51: 1770-1777.

Oligonucleotide primers can be designed or other techniques can be applied to screen lines for mutations/insertions in RAR-encoding sequence(s) or RAR receptor-encoding sequence(s). Through breeding, a plant line may then be developed that is homozygous for the mutated copy of the RAR-encoding sequence(s) or RAR receptor-encoding sequence(s). PCR primers for this purpose may be designed so that a large portion of the coding sequence of the desired sequence is specifically amplified using the sequence of the sequence from the species to be probed (see, for example, Baumann, E. et al. (1998), “Successful PCR-based reverse genetic screens using an En-1-mutagenised Arabidopsis thaliana population generated via single-seed descent”, Theor. Appl. Genet. 97:729 734).

Other RAR or RAR receptor mutants may be isolated from mutant populations or existing germplasm using the distinctive phenotypes characterized in accordance with the present invention (such as modulated root architecture compared to the wild-type plants). That the phenotype is caused by a mutation in RAR-encoding sequence(s) or RAR receptor-encoding sequence(s) or a homologue thereof may then be established by molecular means well known in the art.

RAR or RAR receptor mutants, including mutants heterozygous for the allele, and which may not express the modulated phenotype, may also be screened for, as described herein, and the mutants used for breeding programs to introgress the mutation into homozygous line, or the mutant gene isolated and used in recombinant techniques for generating mutant plants.

While mutants of the present invention may be generated by random mutagenesis (or may already exist), any plant may be recombinantly engineered to display a similar phenotype, for example once the genetic basis of the mutation, such as a mutated RAR-encoding gene, has been determined. For a general description of plant transformation and regeneration see, for example, Walbot et al. (1983) in “Genetic Engineering of Plants”, Kosuge et al. (eds.) Plenum Publishing Corporation, 1983 and “Plant Cell, Tissue and Organ Culture: Fundamental Methods”, Gamborg and Phillips (Eds.), Springer-Verlag, Berlin (1995). See also Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000).

Screening a plant for the presence of at least one mutant allele of a nucleotide sequence encoding a RAR, RAR receptor, or homologue thereof, may comprise analysing DNA of the plant using at least one nucleic acid molecule suitable as a probe or primer which is capable of hybridising to a RAR gene, RAR receptor gene, or homologue thereof under stringent conditions. In a more specific method, the screening method may comprise the use of at least one oligonucleotide primer pair suitable for amplification of a region of the RAR gene, RAR receptor gene, or homologue thereof, comprising a forward primer and a reverse primer to detect the presence or absence of a mutation in said region. The region may comprise the whole RAR gene, RAR receptor gene, or homologue thereof, or may comprise only a portion thereof.

DNA from the plant to be assessed may be extracted by a number of suitable methods known to those skilled in the art, such as are described in a wide range of well known texts, including (but not limited to) Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000), incorporated herein by cross-reference. See also the methods described in Lukowitz, W., Gillmor, C. S. and Scheble, W-R. (2000) “Positional Cloning in Arabidopsis: Why It Feels Good to Have a Genome Initiative Working for You” Plant Physiology 123, 795-805, and references cited therein.

Once suitable DNA has been isolated, this may be analysed for the presence or absence of a mutation by any suitable method as known in the art, and which method/strategy is employed may depend on the specificity desired, and the availability of suitable sequences and/or enzymes for restriction fragment length polymorphism (RFLP) analysis. Suitable methods may involve detection of labelled hybridisation product(s) between a mutation-specific probe and at least a portion of the RAR gene, RAR receptor gene, or homologue thereof or, more typically, by amplification of at least a portion of the RAR gene, RAR receptor gene, or homologue thereof using either a primer and suitable probe, or using a pair of primers (forward and reverse primers) for amplification of a specific portion of the RAR gene, RAR receptor gene, or homologue thereof, followed by either direct partial and/or complete sequencing of the amplified DNA, or RFLP analysis thereof. Suitable primer pairs for amplifying portions of RAR genes from Medicago truncatula are provided in Table 1—other suitable primers or primer pairs for analysing RAR genes or homologues thereof may be designed based on any one of SEQ ID NOs: 3, 5, 7, 9, 11, 13, 15, 17, 19, 21, 23, 25, 27, 29, 31, 33, 35, 364, 366, 368, 370, 372, 374, 376, 378, 380, 382, 384 or 386.

The methods and reagents for use in a PCR amplification reaction are well known to those skilled in the art. Suitable protocols and reagents will largely depend on individual circumstances. Guidance may be obtained from a variety of sources, such as for example Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein and Ausubel et al. (eds) Current Protocols in Molecular Biology, John Wiley & Sons (2000), incorporated herein by cross-reference.

A person skilled in the art would readily appreciate that various parameters of the PCR reaction may be altered without affecting the ability to amplify the desired product. For example the Mg²⁺ concentration and temperatures employed may be varied. Similarly, the amount of genomic DNA used as a template may also be varied depending on the amount of DNA available.

Other methods of analysis of the amplified DNA to determine the presence or absence of a mutation are well known to those skilled in the art. For instance, following digestion of the amplified DNA with a suitable restriction enzyme to detect a mutation in a RAR gene, RAR receptor gene, or homologue thereof, the DNA may be analysed by a range of suitable methods, including electrophoresis. Of particular use is agarose or polyacrylamide gel electrophoresis, a technique commonly used by those skilled in the art for separation of DNA fragments on the basis of size. The concentration of agarose or polyacrylamide in the gel in large part determines the resolution ability of the gel and the appropriate concentration of agarose or polyacrylamide will therefore depend on the size of the DNA fragments to be distinguished.

Detection and/or determination of the existence of a mutation in a RAR gene, RAR receptor gene, or homologue thereof may be aided by computer analysis using any appropriate software. Suitable software packages for comparison of determined nucleotide sequences are well known in the art and are readily available.

Methods for modulating root architecture in plants, as compared to wild-type plants, may also be developed along any of the lines described above based on RKN RGFs (see SEQ ID NOs: 416 or 417) and their encoding sequences, and/or RKN IDAs (see SEQ ID NO: 418) and their encoding sequences.

### Inhibition of RKN-Modulation of Root Architecture

According to an important aspect, the present invention relates to methods for inhibiting root architecture modulation by root knot nematodes (RKNs). As mentioned previously, RKNs are obligate parasites infecting a broad spectrum of vascular plants, causing crop yield losses estimated at more than USD50 billion per annum, yet effective RKN control strategies are limited. Similarities observed during the present studies between the bumps formed on developing roots in plants, overexpressing RARs, development of nodules in legumes, and development of galls formed on plant roots as a result of RKN infestation led to our discovery that RKNs, but not other nematodes possess RAR sequences. The ‘knots’ induced by RKN appear to incorporate two developmental responses: gall formation, which involves limited proliferation of mainly cortical cells in a circumferential manner around the root and the formation of RKN feeding sites in the plant vascular tissue, which include giant cells. RKN and Medicago RAR peptides only induce the circumferential cell proliferation (CCP sites), not RKN feeding sites. The apparent unique distribution of RAR only in root knot; but not other nematode genomes (including cyst nematodes and other plant associating nematodes), and their expression during RKN infection, suggests that RAR may be required as a virulence determinant.

Thus, it is contemplated that modulation of plant root architecture by RKNs may be inhibited by inhibiting expression of RKN RARs, and/or by interfering with RKN RAR interaction with plant RAR receptors or by mimicking endogenous plant RAR ligands and thereby disrupting normal RAR function. This may be achieved by a number of means, including:

(a) contacting the root zone of said plant with a root knot nematode RAR signaling antagonist;

(b) introducing at least one exogenous nucleic acid into one or more plant cells which results in modulated RGF expression in a root knot nematode;

(c) introducing at least one mutation or exogenous nucleic acid into one or more plant cells which results in modulated affinity of one or more root knot nematode RARs for plant RAR receptors, which modulated affinity arises through modifications in the plant RAR receptor(s); or

(d) introducing at least one exogenous nucleic acid into one or more plant cells which results in expression of one or more root knot nematode RGF binding agents or antagonists by said cell(s).

An RKN RAR signaling antagonist for use in a method of the present invention may be any substance that interferes with the physiological response usually triggered by an RKN RAR when it binds to a plant RAR receptor, or which interferes with binding of the RKN RAR to a plant RAR receptor. For example, a RKN RAR antagonist may be a substance that binds to a RKN RAR to inhibit interaction between the RKN RAR and the plant RAR receptor, which interaction would trigger a root architectural response. RKN RAR antagonists may include antibodies to RKN RARs.

An antibody (or fragment thereof) may be raised against a RKN RAR or immunogenic portion thereof using any suitable method known in the art. For instance, a monoclonal antibody, typically containing Fab portions, may be prepared using the hybridoma technology described in Antibodies-A Laboratory Manual, Harlow and Lane, eds., Cold Spring Harbor Laboratory, N.Y. (1988), the disclosure of which is incorporated herein by reference. Any technique that provides for the production of antibody molecules by continuous cell lines in culture may be used. Suitable techniques include the hybridoma technique originally developed by Kohler et al., Nature, 256:495-497 (1975), the trioma technique, the human B-cell hybridoma technique [Kozbor et al., Immunology Today, 4:72 (1983)], and the EBV-hybridoma technique to produce human monoclonal antibodies [Cole et al., in Monoclonal Antibodies and Cancer Therapy, pp. 77-96, Alan R. Liss, Inc., (1985)]. Immortal, antibody-producing cell lines can be created by techniques other than fusion, such as direct transformation of B lymphocytes with oncogenic DNA, or transfection with Epstein-Barr virus. See, e.g., M. Schreier et al., “Hybridoma Techniques” (1980); Hammerling et al., “Monoclonal Antibodies and T-cell Hybridomas” (1981); Kennett et al., “Monoclonal Antibodies” (1980); wherein the disclosures of each of these citations are also incorporated herein by reference.

For convenient production of adequate amounts of antibody(ies), these may be manufactured by batch fermentation with serum free medium, and then purified via a multistep procedure incorporating chromatography and viral inactivation/removal steps. For example, the antibody may be first separated by Protein A affinity chromatography and then treated with solvent/detergent to inactivate any lipid enveloped viruses. Further purification, typically by size-exclusion, reverse-phase, anion and/or cation exchange chromatographies, may be used to remove residual undesired contaminants such as proteins, solvents/detergents and nucleic acids. The antibody(ies) thus obtained may be further purified and formulated as desired.

These antibodies can include but are not limited to polyclonal, monoclonal, chimeric, single chain, Fab fragments, and a Fab expression library.

A monoclonal antibody refers to an antibody secreted by a single clone of antibody-producing cells and which is monospecific for a particular antigen or epitope. Therefore, a monoclonal antibody displays a single binding affinity for any antigen with which it immunoreacts.

There are also various procedures known in the art which may be used for the production of polyclonal antibodies to a RKN RAR or immunogenic portion thereof. For example, one or more host animals can be immunised by injection with the relevant polypeptide, or a derivative (e.g., fragment or fusion protein) thereof. Suitable hosts include, for example, rabbits, mice, rats, sheep, goats, etc.

The antibody (or fragment thereof) will have a binding affinity or avidity. This binding affinity or avidity may be greater than about 10⁵ M⁻¹, such as greater than about 10⁶M⁻¹, greater than about 10⁷M⁻¹, or greater than about 10⁸M⁻¹.

Rather than apply antibodies to the root zone of a plant, it may be advantageous to transform a plant to produce antibodies specific for RKN RARs. Once monoclonal antibodies have been raised against a RKN RAR, by methods referred to above, sequences encoding the desired antibody component(s) may be cloned from cDNAs, assembled into plant expression vectors, and transfected into plant cells. Methods for expressing antibodies in plant cells have been well described in the art. See, for example, Stoger E et al (2004), “Antibody production in transgenic plants”, in Lo BKC (ed) “Methods in Molecular Biology. Antibody Engineering: Protocols and Methods” (2^{nd} edn). Humana Press Inc., NJ, pp 301-318; Fischer R et al (2003), “Production of antibodies in plants and their use for global health”, Vaccine 21: 820-825; Wycoff K L (2005), “Secretory IgA antibodies from plants”, Curr Pharm Des. 11(19): 2429-37; and Prins M et al (2008), “Strategies for antiviral resistance in transgenic plants”, Molecular Plant Pathology 9(1): 73-83.

According to an embodiment of the invention, the antibody(ies) are secreted by the transformed plant cells.

Other methods for inhibiting root architecture modulation by RKNs may include introducing into plant cells exogenous nucleotide sequences coding for nucleotide molecules which, once taken up or incorporated by RKNs, suppress expression of RKN RARs. Such nucleotide sequences may encode antisense, RNAi or microRNA molecules, as described previously. Suitable constructs and vectors and transformation techniques for introducing targeting sequences, or nucleotide constructs for expressing them, as well as methods for regenerating plants from transformed cells have already been discussed above.

Yet further methods for modulation of root architecture may comprise decreasing RKN RAR signaling by modulation of the affinity of one or more RKN RARs for plant RAR receptor(s) through mutation of the plant RAR receptor(s), or introducing exogenous sequences coding for one or more plant RAR receptor(s) with less affinity for RKN RAR(s). Methods for introducing mutations into target nucleotide sequences, and screening thereof, have been described further above.

Methods as described above for inhibiting root architecture modulation by RKNs may also be developed based on the RKN root growth factors (RGFs) and IDA peptides disclosed herein. Exemplary RKN RGF peptides are disclosed herein as SEQ ID NOs: 416 and 417, having RGF domains comprising amino acid sequences as shown in SEQ ID NOs: 460 and 461 (Meloidogyne hapla and M. incognita respectively). An exemplary RKN IDA peptide is disclosed herein as SEQ ID NOs: 418, having′ an IDA domain comprising the amino acid sequence as shown in SEQ ID NO: 496 (Meloidogyne hapla).

Another route for inhibiting root architecture modulation by RKNs may be based on the fact that RKNs have been found to secrete a number of analogs of plant signaling peptides, including CLEs, as well as RARs, RGFs and IDAs, as shown by the present studies. All of these peptides are predicted to be secreted, after post-translational modifications of the product translated from the encoding sequence, and after cleavage of signal peptides from the nascent peptides.

A component of SPC (Signal peptidase complex), signal peptidase I exists in RKN genomes and is a contemplated as a target for in nematodes to disrupt the nematode secretory pathway that is critical for their parasitism. Being an early component of the protein secretory pathway in the endoplasmic reticulum (ER), the SPC cleaves signal peptides off nascent polypeptides that are destined for intracellular compartments and the extracellular matrix (Paetzel M et al. 2002 Chem Rev. 102(12):4549-80. Signal peptidases). Engineered disruption of signal peptidase I (SPI) are contemplated to disable proper secretion of extracellular proteins and peptide including RARs, RGFs, CLEs and IDAs, and thus suppress plant parasitise nematodes.

Thus, it is contemplated that modulation of plant root architecture by RKNs may be inhibited by inhibiting a component of a root knot nematode signal peptidase complex. This may be achieved by introducing at least one exogenous nucleic acid into one or more plant cells which suppresses, or the product of which suppresses expression of a component of a root knot nematode signal peptidase complex. According to an embodiment, the component is signal peptidase I, which may comprise a nucleotide sequence as shown in SEQ ID NO: 419, or an amino acid sequence as shown in SEQ ID NO. 420.

Thus, and as described above in the context of RARs and RGFs, methods for inhibiting root architecture modulation by RKNs may include introducing into plant cells exogenous nucleotide sequences coding for nucleotide molecules which, once ingested by RKNs, suppress expression of such components of the RKN SPC. Such nucleotide sequences may encode antisense, RNAi or microRNA molecules, as described previously. Suitable constructs and vectors and transformation techniques for introducing targeting sequences, or nucleotide constructs for expressing them, as well as methods for regenerating plants from transformed cells have already been discussed above.

Plants with Modulated Root Architecture or which Resist Root Architecture Modulation by RKNs

Plants having modulated root architecture, or which resist root architecture modulation by RKNs, obtained by any of the methods described above, are also encompassed within the ambit of the present invention.

Also encompassed are plant parts, including but not restricted to leaves, stems, roots, tubers, flowers, fruits and seeds obtained from such plants.

Preferred forms of the present invention will now be described, by way of example only, with reference to the following examples, including comparative data, and which are not to be taken to be limiting to the scope or spirit of the invention in any way.

### EXAMPLES

### Example 1

### Materials and Methods

### Plant Materials and Growth Conditions

Seeds of M. truncatula cv Jemalong genotype A17 wild-type and M. truncatula 2HA line carrying either GH3 promoter-GUS reporter fusion gene (GH3:GUS) were grown under standard conditions (Holmes P, Goffard N, Weiller G F, Rolfe B G, & Imin N (2008), “Transcriptional profiling of Medicago truncatula meristematic root cells”, BMC Plant Biol 8:21).

### Identification of RARs in Nematodes

All available genome sequence for the plant parasitic nematodes Meloidogyne hapla, M. incognita, and M. chitwoodi, Globodera rostochiensis, Heterodera glycines, Pratylenchus coffeae, Radopholus similis as well as the free-living nematode C. elegans were processed to discover open reading frames between 30 and 150 amino acids long, from ATG to stop, using the program getorf. SignalP was used to search for signal sequences in all resulting ORFs, using both neural network (NN) and Hidden Markov Model (HMM) modes. A custom-made database of ORFs with an identifiable signal sequence was created and searched for the pattern “xfrPTxpGxSPGxGx” (SEQ ID NO: 421) using a double-affine Smith-Waterman algorithm from TimeLogic (TimeLogic DeCypher systems). Resulting matches were hand-curated for conservation of RAR domains as compared to RAR domains found in A. thaliana and M. truncatula.

### Identification of RARs in Plants

The pervasiveness of genes with RAR domains in plant genomes was examined using the conserved 15-amino-acid M. truncatula RAR sequences as queries for BLAST searches (http://blast.ncbi.nlm.nih.gov/Blast.cgi).

RNA Extraction, cDNA Synthesis and qRT-PCR Analysis.

RNA extraction, cDNA synthesis and qRT-PCR analysis was performed as described in L. Kusumawati, N. Imin, M. A. Djordjevic, (2008), “Characterization of the secretome of suspension cultures of Medicago species reveals proteins important for defense and development” J. Proteome Res. 7: 4508. The primers used are listed in Table 1 (see over). Normalization was conducted by calculating the differences between the C_{T} of the target gene and the C_{T} of MtUBQ10 (MtGI accession number TC161574). Normalization for relative quantification for the transcript level of each gene was carried out according to ‘delta-delta method (2). According to the method, the average C_{T} values of the gene of interest from the technical triplicate of a sample is subtracted with the average C_{T} values of the housekeeper gene (MtUBQI0) from the same sample as shown in the formula below: ΔCt=C_{T}^{gene of interest}−C_{T}^{housekeeper gene (MtUBQ10)}. The same calculation was carried out for both the control sample and the sample of study. The ΔC_{T} value obtained from the above calculation was then used to calculate the ‘delta-delta’ Ct value

TABLE 1
Primers used for cloning of M. truncatula RAR gene and the real-time qRT-
PCR analysis. Accession numbers are from either Affymetrix probe IDs or M. truncatula
gene index IDs (compbio.dfci.harvard.edu) or from International Medicago Genome
Annotation (www.medicago.org/genome/IMGAG/) IDs. MtRAR6-9 sequences are from
unannotated sequences.

| | Accession | Forward primer | Reverse primer |
|---|---|---|---|
| Name | number | (5′-3′) | (5′-3′) |
| Gateway cloning | | | |
| MtRAR1 | Mtr.7265.1.S1_at | CACCATGGCTTATAAATTTCAAT | TCAATTTCCAATTTTGTTTTGGT |
| | | ACACAATGA | (SEQ ID NO: 423) |
| | | (SEQ ID NO: 422) | |
| qRT-PCR analysis | | | |
| MtRAR1 | Mtr.7265.1.Sl_at | CCGATGAAGATATCGACGTGAA | GAACTCATTTGTAGTATCCTCAG |
| | | (SEQ ID NO: 424) | TCACAT (SEQ ID NO: 425) |
| MtRAR2 | META519TF | TAGCTCGCATTTGCTTGTTC | GGCTGAATGCTTTGTCTCAA |
| | | (SEQ ID NO: 426) | (SEQ ID NO: 427) |
| MtRAR3 | TC125059 | ACGTTGAGCTCCACCATTTT | GAGCGCTCCACCTCCTATTA |
| | | (SEQ ID NO: 428) | (SEQ ID NO: 429) |
| MtRAR4 | Medtr5g025790.1 | CATGGAGGTGGTGTTTGATG | TTTTCGCCCTACAAGTCCAG |
| | | (SEQ ID NO: 430) | (SEQ ID NO: 431) |
| MtRAR5 | Medtr5g017710.1 | GTGTTGTTTTGAGCCCAAGG | TGTTGGTCGAAAAGCTTCAA |
| | | (SEQ ID NO: 432) | (SEQ ID NO: 433) |
| MtRAR6 | AC233112_1004.1 | GCTCATCATGGAGGGAAGTC | TATGCCCTGGAGATGTAGGC |
| | | (SEQ ID NO: 434) | (SEQ ID NO: 435) |
| MtRAR7 | AC233112_1013.1 | CCGGATGTTGAGGTTTTTGT | GGCCAACTCCAGGACTATGA |
| | | (SEQ ID NO: 436) | (SEQ ID NO: 437) |
| MtRAR8 | AC233112_1014.1 | TCCAACAATATTGCCACCAA | GGGTTGTGGGTCTAAAAGCA |
| | | (SEQ ID NO: 438) | (SEQ ID NO: 439) |
| MtRAR9 | AC233112_1014.1 | TGATGCCAAATCATGGTGTC | GGACTGCTTCCTGGTGTTGT |
| | | (SEQ ID NO: 440) | (SEQ ID NO: 441) |
| MtRAR10 | Medtr5g030490.1 | TCAATGGAAGCATCAAGGTTT | TATATGTCCCACCCCAAGAC |
| | | (SEQ ID NO: 442) | (SEQ ID NO: 443) |
| MtRAR11 | Medtr8g086660.1 | AGCTCCTTCCATTGGCTTTT | CCCCACCAGGACTATGACC |
| | | (SEQ ID NO: 444) | (SEQ ID NO: 445) |
| MtNRT2.5 | Mtr.35456.1.Sl_at | GGAGAAGGAGAAAGGGTCTCA | TCAGAAGGCCTAGTTGAAATG |
| | | (SEQ ID NO: 446) | (SEQ ID NO: 447) |
| MtAGL1 | Mtr.15656.1.S1_at | GAACCGAAGGGAAGCATAA | TGTCGTGCCATACACCTTTT |
| | | (SEQ ID NO: 448) | (SEQ ID NO: 449) |
| MtLBD38* | Mtr.22734.1.S1_at | GCCACGCTACTGTTTTCGTA | GAGCTGGTCTCTGTGGTTCA |
| | | (SEQ ID NO: 450) | (SEQ ID NO: 451) |
| MhRAR10 | Mh_Contig368 | GCACCTCAACCTCCTTTCTGCA | TGTCCATTTACTGGTGGCTTACA |
| | | (SEQ ID NO: 452) | TGG (SEQ ID NO: 453) |
| MtUB010 | TC100142 | AACTTGTTGCATGGGTCTTGA | CATTAAGTTTGACAAAGAGAAAG |
| | | (SEQ ID NO: 454) | AGACAGA (SEQ ID NO: 455) |

according to the formula below: ΔΔC_{T}=ΔC_{T}^{sample of study}−ΔC_{T}^{control}. These values were then used to calculate for the fold differences of each sample by using the following formula: Fold difference=2^{-ΔΔC}_{T}. From the calculation, the control samples were valued close to 1 and all the other samples had a relative value to the controls. These values were then calculated in Excel for their standard error and their P-values using Student's t-test. Three biological (independent root samples), two experimental (independent cDNA synthesis) and three technical repeats (independent real-time PCR) were done for each sample.
Agrobacterium rhizogenes-Mediated Hairy Root Transformation

A PCR fragment corresponding to the full-length open reading frames of MtRAR1 was amplified from M. truncatula cDNA and cloned into the pK7WG2D vector by methods as as described in Karimi M, Inze D, & Depicker A (2002) “GATEWAY vectors for Agrobacterium-mediated plant transformation”, Trends Plant Sci 7(5):193-195. The respective constructs were transformed into A. rhizogene, strain Arqua1 as described in Saur I M, Oakes M, Djordjevic M A, & Imin N (2011), “Crosstalk between the nodulation signaling pathway and the autoregulation of nodulation in Medicago truncatula”, New Phytol, 190(4):865-874. Transgenic roots were identified by the presence of green fluorescent protein (GFP) with an Olympus SZX16 stereomicroscope equipped with a GFP filter unit (Model SZX2-FGFPA, Shinjuku-ku, Tokyo, Japan).

Nodulation with Sinorhizobium meliloti and Assessment of Nodule Numbers

The 3-weeks old transformed hairy-roots plants were first transferred to a modified Fahraeus media without NH₃NO₄ and kanamycin to starve the plants of nitrogen for 4 days. Inoculation with Sinorhizobium meliloti was done as described in Saur I M, Oakes M, Djordjevic M A, & Imin N (2011), “Crosstalk between the nodulation signaling pathway and the autoregulation of nodulation in Medicago truncatula”, New Phytol 190(4):865-874.

### Exogenous Application of Synthetic Peptides

The RAR peptides were synthesized at the Biomolecular Resource Facility, The Australian National University. The 15 amino acid (aa) peptides corresponding to the conserved domains of MtRAR1 (AFQHypTTPGNSHypGVGH and EFQKTNPGHSHypGVGH, where Hyp indicates hydroxy proline residue) and M. hapla MhRAR2 (AFRHypTAPGHSHypGVGH) were synthesized and validated as previously described in Djordjevic M A, et al. (2011), “Border sequences of Medicago truncatula CLE36 are specifically cleaved by endoproteases common to the extracellular fluids of Medicago and soybean”, J Exp Bot 62(13):4649-4659. The root length of wild-type plants was measured four days after transfer to Fahraeus-medium containing the synthetic peptide. For the hormone assays, A17 plants were grown on Fåhraeus-medium for 10 days before transferring to Fåhraeus-medium containing 10⁻⁶ M of the respective phytohormones; 1-aminocyclopropane-1-carboxylic acid (ACC), 6-benzylaminopurine (BAP), gibberellic acid (GA), synthetic analog of strigolactone (GR24), methyl jasmonate (MeJA) and 1-naphthaleneacetic acid (NAA).

### B-Glucuronidase (GUS) Staining and Sectioning

GUS activity was localized in transgenic hairy roots carrying GH3:GUS or MtRAR1:GUS constructs. For the promoter analysis of MtRAR1, the upstream 2.2-kb promoter region of MtRAR1 was amplified by genomic PCR, then cloned into the binary vector pKGWFS7. M. truncatula (A17) roots was transformed with these constructs via Agrobacterium rhizogenes by hairy root transformation method as described in Saur I M, Oakes M, Djordjevic M A, & Imin N (2011), “Crosstalk between the nodulation signaling pathway and the autoregulation of nodulation in Medicago truncatula”, New Phytol 190(4):865-874. Histochemical analysis of GUS gene expression in the transformed plant roots was performed as described in Vitha S, Benes K, Phillips J P, & Gartland K M (1995), “Histochemical GUS analysis”, Methods Mol Biol 44:185-193. Staining and sectioning was performed three times, each time taking roots of four plants, and similar results were obtained each time. Staining was examined with a Nikon SMZ1500 stereomicroscope and photographed with a mounted Digital Sight DS-Ri1 camera (Nikon Inc., Melville, N.Y., USA). Sectioning of the roots was done as described in Saur I M, Oakes M, Djordjevic M A, & Imin N (2011), “Crosstalk between the nodulation signaling pathway and the autoregulation of nodulation in Medicago truncatula”, New Phytol 190(4):865-874.

### Confocal Microscopy

Root samples were fixed in fixative (50% methanol and 10% acetic acid) at 4° C. for overnight, rinsed with water and stained with 10 μg/ml propidium iodide in water at room temperature (avoiding light) until plants were visibly stained (less than 3 h). Then the roots were examined by multiphoton imaging using a LSM 780 confocal microscopy (Carl Zeiss, Jena, Germany).

### Example 2

### Plant RARs

We examined the distribution and function of a multigene family we call RARs (Root Architecture Regulators). Phylogenetic analyses indicate that RAR genes are unique to the genomes of higher plants and RKN, and encode a conserved 15 amino acid RAR domain that is predicted to be secreted. Using expression analysis we show that in the model legume Medicago truncatula, RARs are regulated by lowered N-status and elevated CO₂ and they play an important role in controlling root development and the expression of genes integral to the control of N-status and uptake including ANR1, NRT2.1, NRT2.5 and LBD38. Due to the technological difficulties experienced with knockdown strategies for large multigene families we used over-expression studies to demonstrate that the RAR domain encoding gene, MtRAR1, profoundly affect multiple aspects of root architecture and development including lateral root and nodule formation and root hair development. Superficially, the periodic bumps induced resemble galls, and this is corroborated by confocal imaging.

Ohyama et al (2008) had previously shown that an Arabidopsis gene AtCEP1 (C-terminal encoded peptide) produces a 14 or 15 amino acid secreted ligand that affects primary root growth only (Ohyama K, Ogawa M, & Matsubayashi Y (2008), “Identification of a biologically active, small, secreted peptide in Arabidopsis by in silico gene screening, followed by LC-MS-based structure analysis”, Plant J 55:152-160). AtCEP1 corresponds to AtRAR1, and is thus the historical canonical member of this family. However, since our results place M. truncatula RARs at the crossroads of root development and responses to nutritional cues, we renamed the CEPs to reflect this key function. In addition, our results also point to RAR ligand mimicry by RKNs suggesting a role for these nematode peptides in gall formation.

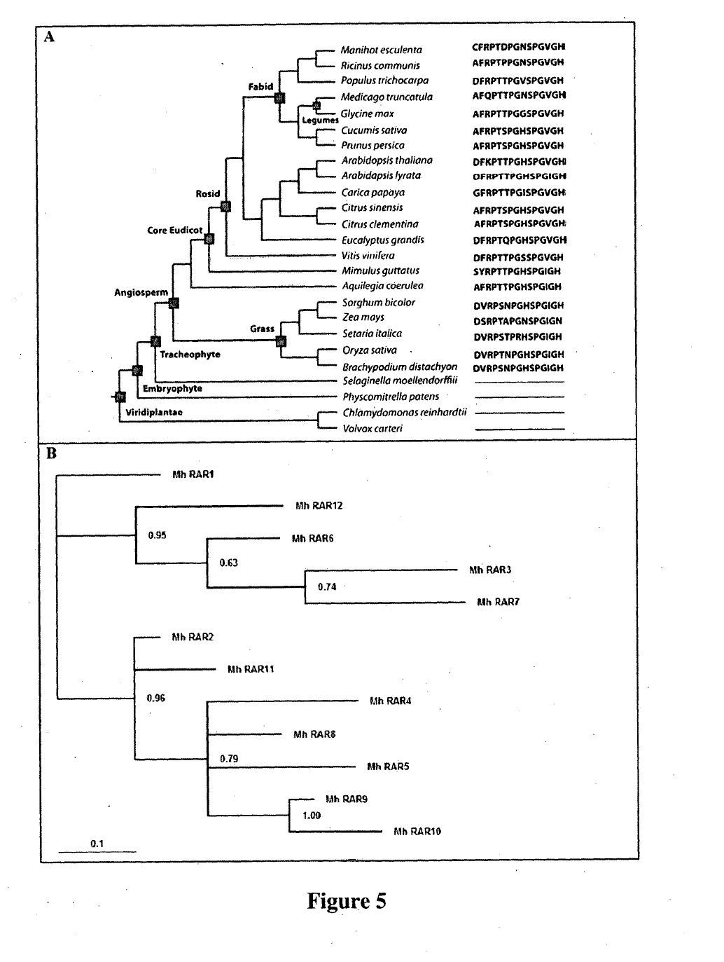

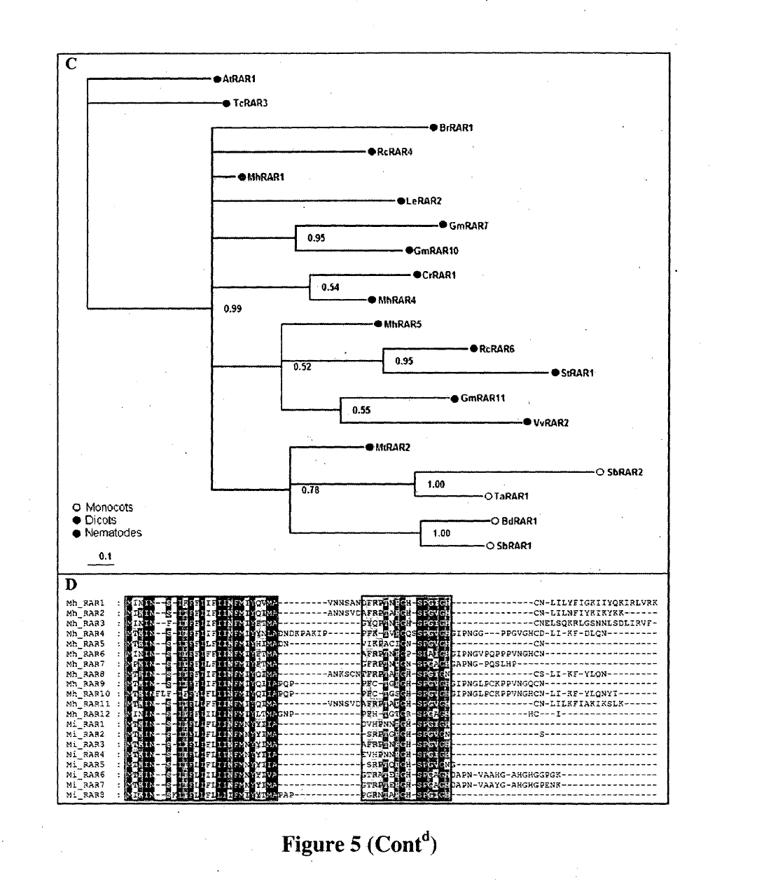

Conserved RAR domains are widely dispersed in angiosperms as multigene families (FIG. 1A, FIG. 5A and SEQ ID NOs: 2, 4, 6, 8, 10, 12, 14, 16, 18, 20, 22, 24, 26, 28, 30, 32, 34 and 36-147 for peptide sequences, and SEQ ID NOs: 1, 3, 5, 7, 9, 11, and 13 for Arabidopsis thaliana RAR-encoding sequences, and SEQ ID NOs: 15, 17, 19, 21, 23, 25, 27, 29, 31, 33 and 35 for Medicago truncatula RAR-encoding sequences).

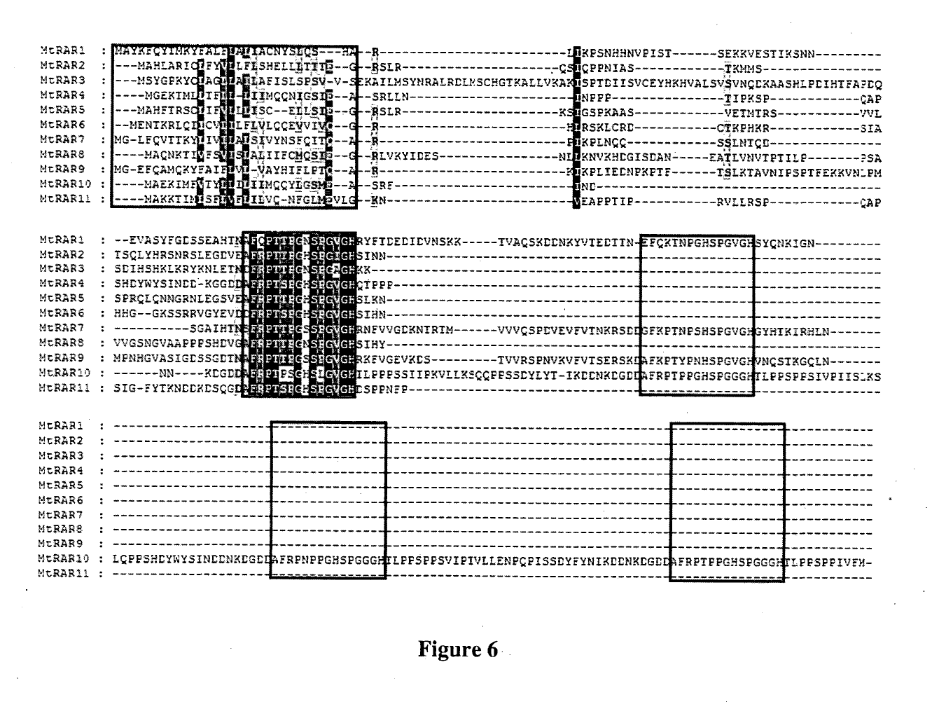

M. truncatula was found to encode eleven RAR loci (see SEQ ID NOs: 15, 17, 19, 21, 23, 25, 27, 29, 31, 33 and 35 for Medicago truncatula RAR-encoding sequences and SEQ ED NOs: 16, 18, 20, 22, 24, 26, 28, 30, 32, 34 and 36 for Medicago truncatula RAR translated peptide sequences). Except for the peptide domains, little sequence conservation exits amongst RAR genes (see FIGS. 1A and 6). However, RAR genes encode an amino-terminal signal peptide or a non-classical secretion signal (see, for example, FIGS. 1B to 1D, 5D and 6), which is a feature of secreted regulatory plant peptide families. A strongly conserved functional RAR sub-domain occurs at the C′-terminus of the RAR domain (FIG. 1A).

RAR genes can encode single or multiple RAR peptides. For example, MtRAR1 (SEQ ID NO: 15) encodes two peptides (see FIG. 1B and SEQ ID NOs: 166 and 167; see also SEQ ED NO: 16 for fully translated sequence), MtRAR10 (SEQ ID NO: 33) encodes four peptides (see SEQ ID NOs: 178 to 181; see also SEQ ID NO: 34 for fully translated sequence) and the poplar gene, PtRAR2, encodes seven (see SEQ ID NOs: 231-237 and SEQ ID NO: 72 for fully translated sequence).

RAR domains in monocots are distinctive to those in dicots (FIG. 1A and FIG. 5A, and see also SEQ ID NOs: 301 to 336—monocot RAR peptide domain sequences—vs SEQ ID NOs: 148 to 300—dicot RAR peptide domain sequences). Monocot RAR peptides universally lack the conserved phenylalanine residue common to dicot RAR peptides, and all dicot RAR domains terminated with histidine whereas monocot RAR domains terminated with histidine or asparagine (FIGS. 1A and 5A). We also found genes encoding RAR-like domains in gymnosperms (white spruce and lodgepole pine—peptide sequences: SEQ ID NOs: 338-350; domain sequences: SEQ ID NOs: 351-363) but not in the evolutionary more primitive plants, Selaginella or mosses (FIG. 1A, Table 2), unlike CLEs (found in Selaginella and moss) or RGFs (found in Selaginella). Angiosperm RAR genes encoded an amino-terminal secretion signal, lacked introns, and consisted of one to seven, 15 amino acid, RAR. Apart from the secretion signals and the RAR domains themselves, plant RAR genes had little other sequence conservation (See, for example, FIG. 1A). The gymnosperm RAR-like domains are different from angiosperm RAR in that they exhibit divergence at the first 6 amino acids and have a highly conserved leucine, instead of proline, at position 7. However, the remaining′ eight carboxyl amino acids of gymnosperm RAR-like domains are strongly conserved with those of angiosperm RAR domains (FIG. 1 A).

### Example 3

### RAR Genes are Found Exclusively in Higher Plants and Root-Knot Nematodes

Apart from higher plants (angiosperms and gymnosperms) only the obligate plant parasitic animals, root knot nematodes (RKNs), were found to encode RAR genes.

```
TABLE 2
Existence of growth regulatory peptide coding
genes in plants and nematodes. A representative
  domain sequence is given for species
  in each Glade.
```

| | RAR (or | RGF |
|---|---|---|
| | RAR-like*) | |
| Moss (Physcomitrella | X | X |
| patens) | | |
| Pteridophyte | X | DYTPVHRKPPINN |
| (Selaginella | | (SEQ ID |
| moellendorffii) | | NO: 462) |
| Gymnosperms | ISPFKPLG | DYSTPGDE |
| (Pinus contorta & | HSPGIGH* | PDRQKN |
| Picea sitchensis) | (SEQ ID | (SEQ ID |
| | NO: 359) | NO: 463) |
| Angiosperms | DFRPTNPG | DYSNPGH |
| (Arabidopsis) | NSPGVGH | HPPRHN |
| | (SEQ ID | (SEQ ID |
| | NO: 148) | NO: 475) |
| Root-Knot | DFRPTNPG | DYTGPKH |
| Nematodes | HSPGIGH | HGPRNN |
| (M. hapla) | (SEQ ID | (SEQ ID |
| | NO: 396) | NO: 460) |
| Cyst Nematodes | X | X |
| (Heterodera glycines) | | |

Eight and twelve RAR genes occur in the genomes of Meloidogyne incognita and M. hapla, respectively (FIG. 5D, Table 2 and SEQ ID NOs: 364, 366, 368, 370, 372, 374, 376, 378, 380, 382, 384 and 386 for M. hapla RAR-encoding sequences, SEQ ID NOs: 365, 367, 369, 371, 373, 375, 377, 379, 381, 383, 385 and 387 for M. hapla RAR peptide sequences, and SEQ ID NOs: 388-395 for M. incognita RAR peptides), but none are found in non-root-knot nematodes including C. elegans or the plant parasitic cyst nematodes (see Table 2).

Like plant RAR genes, each RKN RAR identified encoded a putative amino terminal secretion signal but only a single RAR domain peptide, and some RKN RAR genes (e.g. MiRAR 1, 3 and 4) encode only an amino terminal signal sequence and a RAR domain (FIG. 5D).

There are two types of RKN RARs: type one has flanking sequences between the signal sequence and RAR domain and at the C-terminus end of the RAR domain; type two has no flanking sequences between the signal sequence and RAR domain or at the C-terminus end of the RAR domain (FIGS. 1C&D). The juxtaposition of a signal sequence to a RKN RAR domain would obviate the need for processing of several of the RAR domains by additional protease cleavage. Conservation exists between RKN RAR domains and the RAR domains of plant hosts (FIG. 1A; FIG. 5C to 5D and SEQ ID NOs: 148-336, 351-363 and 396-415).

Because the precise evolutionary history of the RAR genes is not known, it is not possible to ascribe orthology, either within the genus, or with plant RARs. Thus, we named the RKN RARs according to their genome assembly coordinates.

RAR loci are absent from the available non-RKN plant parasitic nematode genomes including those of the soybean cyst nematode (Heterodera glycines), potato cyst nematode (Globodera rostochiensis) and the migratory plant parasitic nematodes (Radopholus similis and Pratylenchus coffeae) as well as C. elegans.

Phylogenetic analysis has shown that different MhRAR are more similar to dicot RAR than to each other. (FIG. 5c). For instance, the RAR domain sequence (AFRPTAPGHSPGVGH) of M. hapla MhRAR2 and MhRAR11 was identical to RAR domains of Euphorhia esula (Green spurge; EeRAR2.1, 2.2, 2.3 and 2.5), and the RAR domain-sequence (AFRPTAPGHSPGVGH) of M. incognita MiRAR3 was identical to the RAR domains in Ricinus communis (castor oil plant; RcRAR3 and RcRAR7) and Jatropha curcas (physic nut; JcRAR1). This result may point to RKN and plant RARs sharing an overlapping functional space.

RKN RAR peptides exhibit remarkable similarity to plant RAR peptides (FIGS. 1A and 5), and an overall consensus sequence based on the RAR domains (plant and RKN) may be represented as a 14 to 15 amino acid peptide represented as (X₁)X₂X₃X₄X₅X₆PGX₉SPGX₁₃GX₁₅ (SEQ ID NO:459). Typically, the peptide will comprise 15 amino acids. Aspartic acid, glycine, proline and alanine are typically present at position X₁, although serine and valine, and to a lesser extent other amino acids may be present at this position. Phenylalanine or valine are typically present at position X₂, although threonine, serine, alanine, lysine and tyrosine are often also found at this position, with other amino acids occasionally being observed. Arginine is the predominant amino, acid found at position X₃, especially in monocots and RKNs, proline is the predominant amino acid found at position X₄, threonine, serine or glycine predominantly at position X₅, threonine, alanine or asparagine at position X₆, asparagine or histidine is predominant at position X₉, isoleucine, alanine or valine predominant at position X₁₃ and asparagine or histidine is predominant at position X₁₅. While amino acid substitutions have been observed at positions 7, 8, these are infrequent (and only proline observed at position 7 in monocots, and isoleucine, serine, asparagine and glutamine observed at position 8 in dicots and RKNs). The SPG motif at positions 10-12 is particularly strongly conserved, especially in monocots and RKNs, with very few substitutions being observed in dicots, and only rare substitutions (arginine or threonine) have been observed at position 14, in monocots and dicots.

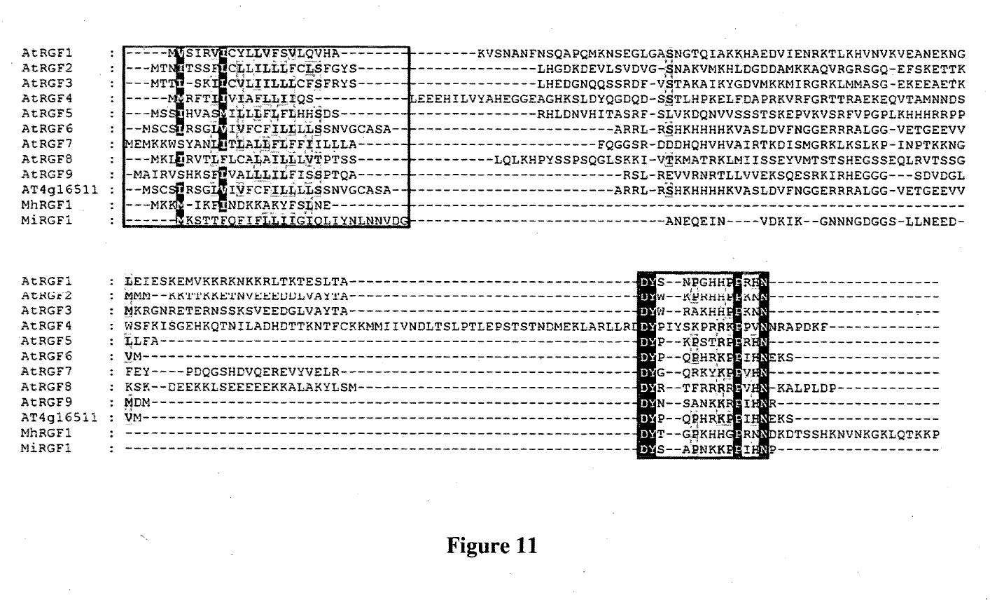

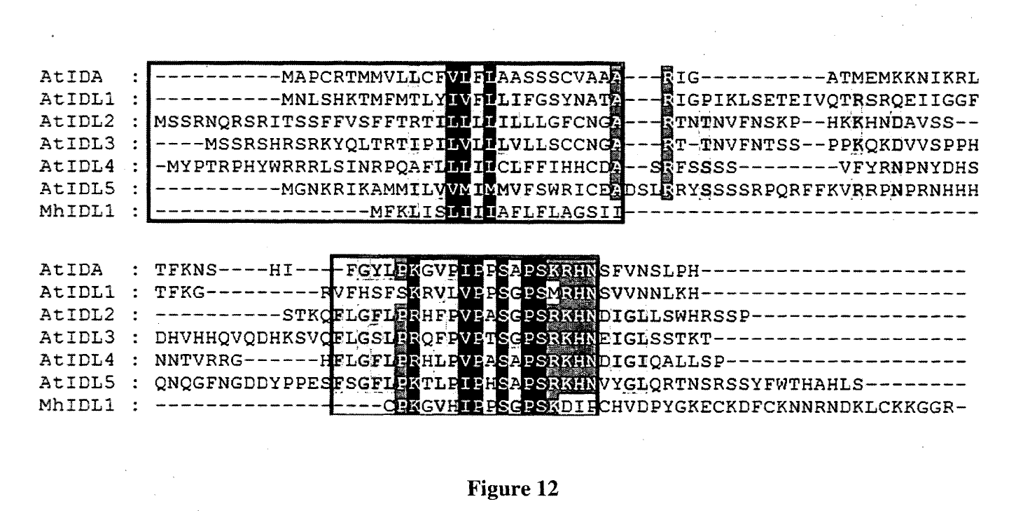

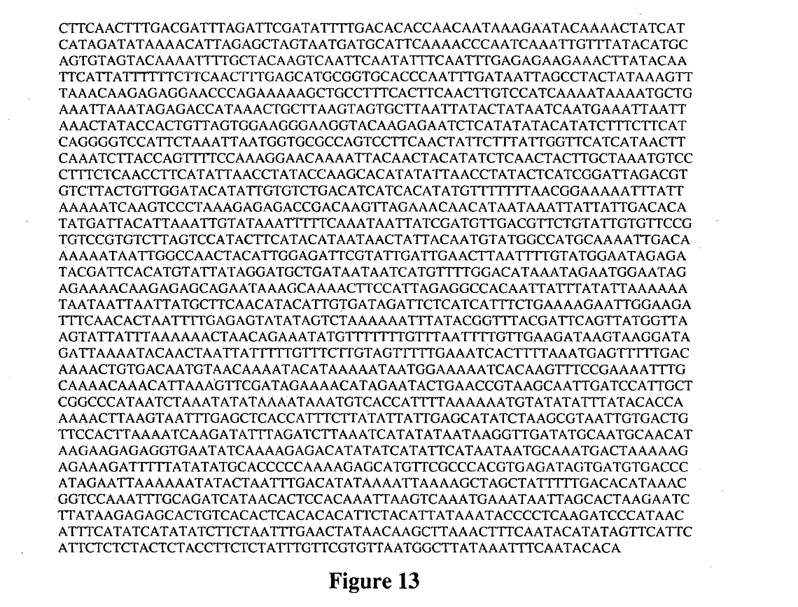

Surprisingly, RKNs were also found to have genes encoding root growth factors (RGFs; see FIG. 11 and SEQ ID NOs: 416 and 417 for full peptide sequences for M. hapla and M. incognita, respectively, as well as Table 2 and SEQ ID NO:460 and 461 for the RKN RGF peptide domain sequences) and Inflorescence Deficient in Abscission peptides (IDAs; see FIG. 12 and SEQ ID NO: 418 for full peptide sequence for M. hapla and SEQ ID NO:496 for the RKN IDA peptide domain sequence), and were known to secrete CLEs. Unlike nematode CLE and RGF genes, RAR gene copy number expanded in RKNs. RNA sequence analysis of galls at three weeks post-infection revealed differential transcriptional activity of each of the Meloidogyne hapla RARs. This observation was confirmed for MhRAR4 (SEQ ED NO: 371) and MhRAR10 (SEQ ID NO: 383) by real-time quantitative reverse transcription PCR, showing them to be expressed at 3-5% relative to the control MhGADPH.

Our studies show that RAR or RAR-like genes occur only in higher plants (angiosperms and gymnosperms) and RKNs. Central to the obligate parasitism of diverse higher plants by RKNs is their ability to subvert intrinsic developmental pathways to enable gall formation. The periodic bumps induced by over-expressing MtRAR1 or the ligands of MtRAR1 or M. hapla MhRAR2, outwardly resemble galls and this supports RAR peptides being bioactive. RKN RAR ligands most likely mimic plant RARs and co-opt plant RAR-dependent pathways during infection and gall formation. RKN RAR expression during gall formation and the tight distribution of RAR loci in the RKN genomes supports this.

M. truncatula bumps initiate in the zone of elongation. Bumps that lack arrested lateral roots arise due to periodic induction of cell divisions in the pericycle, cortical and epidermal cell layers and take ˜10 hours to form. Although the periodic bumps resemble galls and plant cell division is induced during gall formation, bumps do not contain the giant cells induced at RKN feeding sites in the stele. Hence, secreted RKN RARs could play an important role in establishing galls but they play no obvious role in giant cell formation. The known RKN RARs more closely resemble dicot RARs and this may in part explain why in monocots RKN fail to elicit large galls surrounding the obligatory giant cells.

We found that RKN genomes also encode RGF, IDA (inflorescence deficient in abscission) and CLE peptides. Thus, RKNs are unique in that they encode at least four distinct plant-like regulatory peptides that may work in concert during gall formation. Adding to this potential for RKN to produce diverse regulatory ligands is the role of proteolytic modification by the host. Those RKN RARs with the signal sequence directly juxtaposed to the RAR motif are presumably introduced into the apoplast as mature peptides, thereby avoiding any regulatory control by the host and any additional cleavage (beyond removal of the signal sequence) by the RKN itself. By contrast, plant RARs require proteolytic cleavage as an additional regulatory step. Analogous processing is presumably required for those RKN RARs with sequences flanking the RAR peptide. The recent finding that plant proteolytic processing machinery is active on cyst nematode CLEs (Guo Y, Ni J, Denver R, Wang X, & Clark S E (2011), “Mechanisms of molecular mimicry of plant CLE peptide ligands by the parasitic nematode Globodera rostochiensis”, Plant Physiol 157:476-84) provides a precedent.

All twelve M. hapla RAR genes lie in two clusters approximately 100 kb apart. Correlating sequence similarity of the deduced MhRAR proteins with the genomic location and orientation of their genes points to an inverted duplication as an early event in expansion of the RAR family. Such events have been implicated as nucleation events in gene family amplification (Ruiz I C & Wahl G M (1988), “Formation of an inverted duplication can be an initial step in gene amplification”, Mol Cell Biol 8:4302-4313). Analysis of three representative MhRARs in relation to plant RARs Clearly places the MhRARs with dicot RARs, including MhRAR5 which does not have the conserved phenylalanine residue in the second position of the RAR peptide (FIGS. 5C, 5D). The three M. hapla RARs used in the phylogenetic reconstruction do not cluster together, suggesting similar degrees of diversity within the species as is found amongst the plant RARs and this implies functional significance. Collectively, the evidence points to an ancient horizontal gene transfer (HGT) from dicot to the nematode as a mode of acquisition of RARs in RKN. To our knowledge, this is the first example of a plant-parasitic nematode having acquired a dicot gene.

### Example 4

### M. truncatula RAR Genes are Regulated by N-Levels, Elevated CO₂ and Show Differential Expression in Lateral Organs

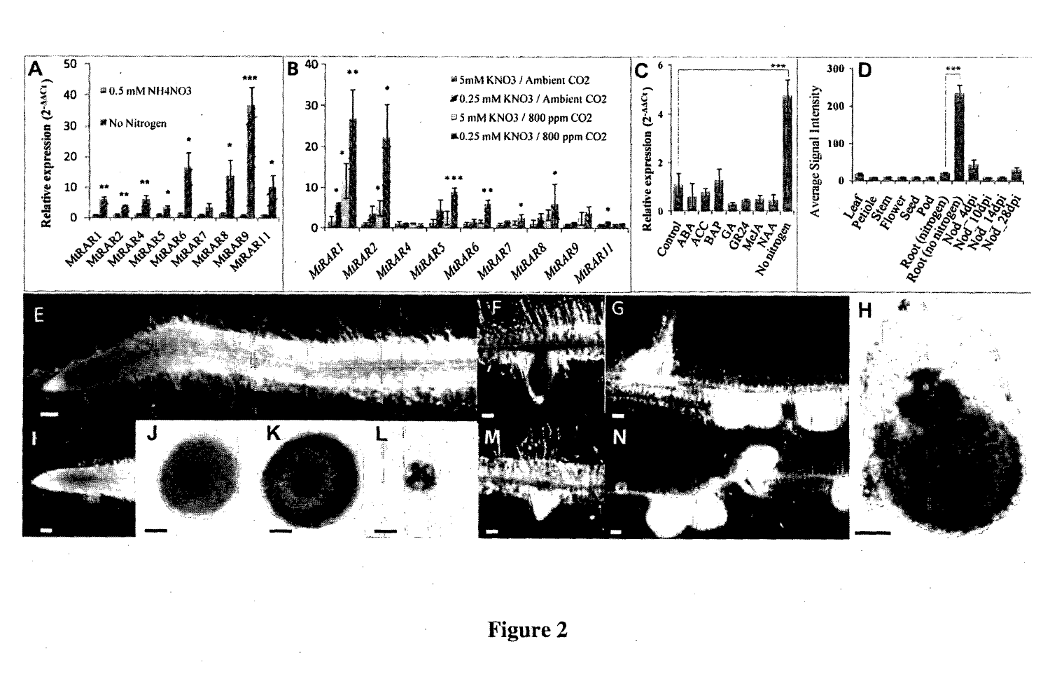

We examined RAR transcript levels in M. truncatula by real-time quantitative reverse transcription PCR (qRT-PCR). Eight of eleven MtRAR genes (MtRAR1, MtRAR2, MtRAR4, MtRAR5, MtRAR6, MtRAR8, MtRAR9 and MtRAR11) were significantly induced between 4 and 36-fold by shifting plants to nitrogen starvation conditions for four days (FIG. 2A). The available microarray data for MtRAR1 is consistent with our findings; MtRAR1 is up-regulated in M. truncatula roots after the imposition of a nitrogen limitation (FIG. 2D).

Recently, elevated CO₂ was shown to inhibit nitrate assimilation in wheat and Arabidopsis, which may lead to a less-than-expected stimulation of plant growth (Bloom A J, Burger M, Rubio Asensio J S, & Cousins A B (2010), “Carbon dioxide enrichment inhibits nitrate assimilation in wheat and Arabidopsis”, Science 328:899-903). An examination of RAR gene expression in M. truncatula grown with 800 ppm CO₂ under different nitrate regimes showed that MtRAR1, MtRAR2, MtRAR5, MtRAR6 and MtRAR8 were highly up-regulated by a combination of elevated CO₂ and low nitrate (0.25 mM KNO₃) (FIG. 2B). MtRAR1 and MtRAR2 were up-regulated by elevated CO₂ even in the presence of 5 mM nitrate. MtRAR1 and MtRAR11 were also up-regulated under low nitrate. Although MtRAR1 was highly responsive to alterations to N and C levels it did not respond to phytohormones (FIG. 2C).

Given the responsiveness of MtRAR1 expression to environmental conditions, this gene was explored further by fusing the 2 kb MtRAR1 promoter sequence to GUS, yielding proMtRAR1:GUS. N-starved roots showed strong GUS staining near root tips and in the vascular tissue (FIG. 2E) but subdued staining occurred in high-N grown roots (FIG. 2I, 2M). Serial sections through the root tip region (FIG. 2J to 2L), showed GUS staining localised in the lateral root cap and apical meristem region (FIG. 2 K) and, in the basal meristem/root elongation region, in cells destined to be vasculature (FIG. 2K). MtRAR1 did not express in columella cells (FIG. 2E, 2I, 2F, 2M). In mature roots staining occurred in the procambium and pericycle (FIG. 2L) but phloem, cortical or epidermal cells were not stained (FIG. 2L).

Subdued GUS staining occurred in roots grown with 5 mM nitrate after a 24-hour incubation in GUS substrate whereas GUS staining in low N-grown plants was already apparent after 2 hours (FIGS. 2I and 2M). Although ProMtRAR1:GUS staining was visible during lateral root initiation and emergence under N-starved conditions (FIG. 2F) it became progressively weaker during nodule development induced by the nitrogen fixing Sinorhizobium meliloti strain WSM1022, (FIG. 2G then FIG. 2N, then FIG. 2H). In roots containing one week old WSM1022 nodules, where leghemoglobin was already apparent, GUS expression occurred in zone II of the developing nodules (FIG. 2 H). However, localised suppression of ProMtRAR1:GUS expression was evident in proximal vascular tissue (FIG. 2G, 2H). As the pink nodules approached two weeks of age, ProMtRAR1:GUS expression was further suppressed in WSM1022 nodules (FIGS. 2G, compared to 2H and 2N) and the surrounding and supporting root vasculature near nodules but notably, not in nearby emerging lateral roots (FIG. 2N). Microarray data also shows negligible MtRAR1 expression during nodule initiation and formation (FIG. 2D).

In contrast to the results obtained with plant inoculated with WSM1022, ProMtRAR1:GUS staining was strongly retained in nodules induced by the model S. meliloti strain Sm1021. At day 7, there was no observable leghemoglobin production indicating that little or no nitrogen fixation occurred under the conditions used (no AVG added to roots) at this time. In two week old Sm1021 nodules, ProMtRAR1:GUS expression was reduced concurrently with the appearance of leghemoglobin. The N-starvation induction of MtRAR1 and the gradual decrease in MtRAR1 expression in one and two week old Sm 1021 induced nodules was reflected in. M. truncatula gene atlas microarray data which showed that MtRAR1 was induced predominantly by nitrogen starvation in the roots but decreased in the developing nodules induced by Sm1021.

We hypothesised that the gradual reduction in MtRAR1 expression in nodules induced by WSM1022 compared to Sm1021 was due in part to the earlier induction nitrogen fixation in WSM1022 nodules. To examine this and to investigate the effect of different levels of nitrogen sources on MtRAR1 expression, we measured the relative expression of MtRAR1 after exposure of plants to two forms of inorganic (nitrate or ammonium) and organic (asparagine or glutamine) nitrogen. Given the results in FIG. 2B, and genevestigator analyses indicated that high CO₂ was an abiotic factor that altered RAR gene expression in Arabidopsis, the experiment was also conducted under ambient and high CO₂ conditions.

MtRAR1 was significantly up-regulated at low N levels at ambient CO₂ when nitrate, asparagine or glutamine was supplied as the sole nitrogen source. However, elevated N levels suppressed expression, with the strongest suppression by asparagine and glutamine at ambient CO₂ level. This is consistent with the suppression of MtRAR1 expression in more mature nodules where assimilation of N resulting from nitrogen fixation is known to result in asparagine and glutamine which are the main products of nitrogen fixation. In addition, higher CO₂ further up-regulated MtRAR1, even at high nitrogen levels, when asparagine was supplied the sole nitrogen source. Consistent with the results in FIG. 2B, the highest up-regulation of MtRAR1 resulted from the combination of high CO₂ and low nitrogen for all nitrogen sources. Statistical analysis showed significant interactions between CO₂ levels and nitrogen levels for nitrate and asparagine, although a similar trend was observed for the other two nitrogen sources, which indicated that low N and high CO₂ independently regulated MtRAR1 expression.

The studies described herein show that M. truncatula RAR genes respond to changed N and CO₂ levels, suggesting that RARs may be involved in regulating responses to changes in C:N. Low nitrate and high CO₂ significantly elevates five of nine MtRAR genes and shifting plants to N-starvation conditions significantly induced eight MtRAR genes. MtRAR1 is unique in responding to low nitrate or N-starvation under ambient CO₂ and to elevated CO₂ in high nitrate. These results point to RARs playing an important regulatory role in responses to environmental cues. MtRAR1 may mediate at least some of its effects by regulating key transcription factors (MtAGL1 and MtLBD38), and/or through the important transporter MtNRT2.5. In Arabidopsis, ANR1 (which is very similar to MtAGL1) and LBD38, regulate N-limitation responses and lateral root formation by controlling the transcription of key nitrate transporters (Gan Y, Filleur S, Rahman A, Gotensparre S, & Forde B G (2005), “Nutritional regulation of ANR1 and other root-expressed MADS-box genes in Arabidopsis thaliana”, Planta 222:730-742; Okamoto M, Vidmar J J, & Glass A D (2003), “Regulation of NRT1 and NRT2 gene families of Arabidopsis thaliana: responses to nitrate provision”, Plant Cell Physiol 44:304-317). MADS box transcription factors are implicated in forming lateral root primordia (Burgeff C, Liljegren S J, Tapia-Lopez R, Yanofsky M F, & Alvarez-Buylla E R (2002), “MADS-box gene expression in lateral primordia, meristems and differentiated tissues of Arabidopsis thaliana roots”, Planta 214:365-372), the promotion of lateral root growth under N-limiting conditions (Gan et al (2005), supra). and have an involvement in tuberous root initiation (Ku A T, Huang Y S, Wang Y S, Ma D, & Yeh K W (2008), “IbMADS1 (Ipomoea batatas MADS-box 1 gene) is involved in tuberous root initiation in sweet potato (Ipomoea batatas)”, Ann Bot 102:57-67). Recently an ABA-regulated LBD gene was implicated in lateral root formation in M. truncatula (Ariel F D, Diet A, Crespi M, & Chan R L (2010), “The LOB-like transcription factor Mt LBD1 controls Medicago truncatula root architecture under salt stress”, Plant Signal Behav 5:1666-1668). The unresponsiveness of MtRAR1 to phytohormones, especially ABA, and the ability of MtRAR1 to control important MADS box and LBD transcription factors suggests that MtRAR1 is pivotal to the integration of environmental cues and root architecture in higher plants. The altered distribution of auxin at bump sites suggests this is downstream of RAR regulation.

### Example 5

### Ectopic Expression of MtRAR1 Profoundly Alters Root Architecture

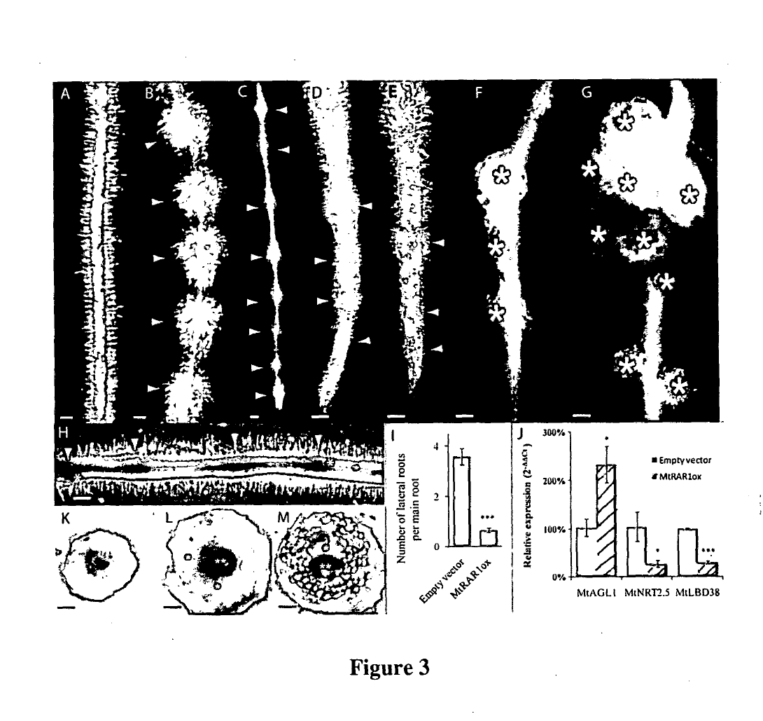

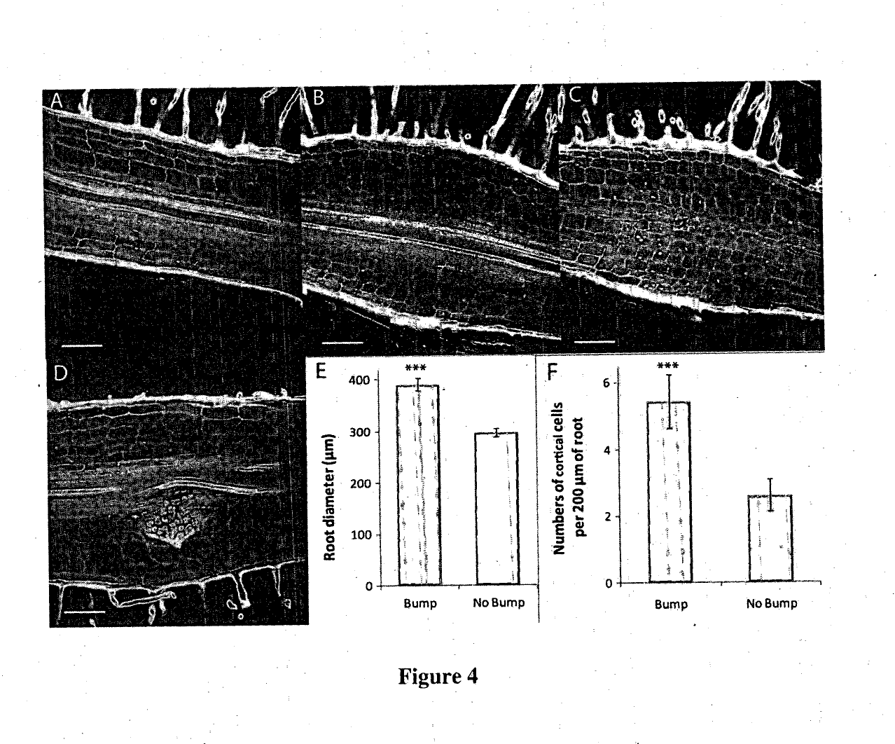

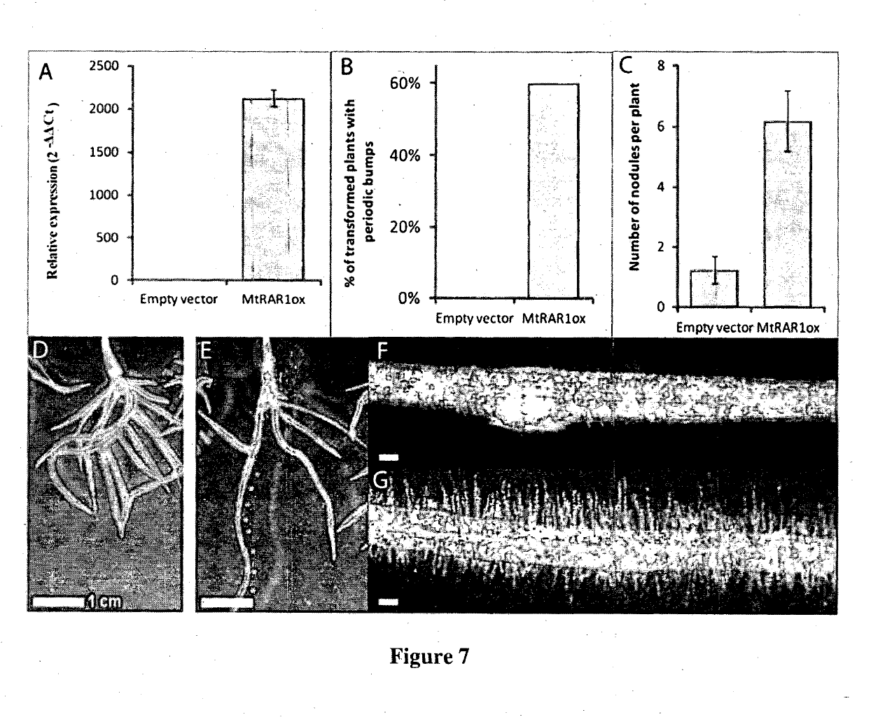

MtRAR1 was over-expressed in M. truncatula roots using the CaMV 35S promoter and confirmed by qRT-PCR (FIG. 7A). The MtRAR1ox roots exhibited five root architecture phenotypes; First, lateral root numbers were significantly reduced 4-fold compared to controls (FIGS. 3I, 7D and 7E) but the growth of the earliest forming hairy roots was not noticeably affected. Second, approximately 60% Of the transformed roots bearing MtRAR1ox formed multiple periodic “bumps”, typified by circumferential cell proliferations (CCP), at the expense of lateral roots (FIGS. 3B, 3C and 3H, 4C, 4D, 7B and 7E). The CCP sites exhibited increased root hair density (FIG. 3B, 3D, 3E). The periodicity was determined by scoring the number of CCP sites over time in several independent measurements under different conditions and it was found to be approximately 24 hours. Maximum CCP site generation was induced by the application of the 15 AA MtRAR1 and MhRAR2 peptides with their expected post translational modifications: all RAR peptide structural variants tested imparted significantly lower, or no, CCP sites. This included a RAR1 peptide with a C-terminal deletion of Histidine; it did not impart any detectible phenotype and was indistinguishable from untreated wild type plants. The timing of the appearance of the youngest CCP sites and their proximity to the root tip showed that they initiated near the basal meristem (FIG. 3C, 3E). Third, approximately 20% of bump sites sectioned contained arrested lateral roots (FIGS. 3H, L-M and 4D) that lacked connection to the root vasculature (FIG. 4D). Fourth, increased root hair development, which is an environmentally-sensitive development pathway (see Schiefelbein J, Kwak S H, Wieckowski Y, Barron C, & Bruex A (2009), “The gene regulatory network for root epidermal cell-type pattern formation in Arabidopsis”, J Exp Bot 60:1515-1521), occurred on bump surfaces but was reduced between bumps (FIGS. 3B-C and 7E). Interestingly, MtRAR1ox roots growing through the agar support medium had fewer and shortened root hairs (FIG. 7F). Finally, MtRAR1ox roots formed 6-fold more nodules (FIGS. 3G and 7C) compared to controls.

Confocal microscopy (FIG. 4) indicated that CCP sites induced by MtRAR1 over-expression were 80 μm wider than the normal root diameter (FIG. 4B to 4E), and showed that the increased root girth at CCP sites was attributable to limited cortical, epidermal and pericycle cell division (FIGS. 4E and 4F) and the formation of an extra cortical cell layer. The nucleus was centrally located in many CCP cells (FIG. 4C). Counting cells over a 200 μm² area at several CCP sites indicated a more than two-fold increase in cortical cell number (FIG. 4F). Serial vibratome sectioning of over 60 plants containing an average of 6 CCP sites each showed no evidence of lateral root initiations at the majority of CCP sites (80%) (e.g. FIG. 4B, 4C). Serial sectioning and confocal microscopy showed that the intervening root segments between the CCP sites showed normal cell patterning and root girth (FIG. 4A). We tested the possibility that CCP sites represented sites where lateral roots would have emerged in the absence of RAR treatment. To do this CCP sites were marked with a black dye and then the root tip was excised (3 mm of tissue was removed only) to release apical dominance. This resulted, within 24-48 h, in lateral roots emerging from CCP sites, but never between them. On average, approximately 24% of the CCP sites formed lateral roots when the root tip was cut. Although a small number of CCP sites generated lateral roots, lateral root number was still not fully restored to the number occurring on plants not exposed to RAR. Therefore, lateral root initiation and/or emergence was promoted by removal of the RAM in RAR treated plants but not restored fully to wild type levels. This rapid emergence of lateral roots from CCP sites contrasts strongly with the persistent inhibition of lateral root initiation and emergence seen on plants on RAR treated plants or where MtRAR1 was over-expressed. Plants exposed to non-functional RAR peptide variants, in which non-conservative substitutions were made to highly conserved amino acids, did not show the persistent inhibition of lateral root emergence.

MtRAR1 over-expression in M. truncatula plants containing GI-13:GUS, an auxin responsive reporter construct (Mathesius U, et al. (1998), “Auxin transport inhibition precedes root nodule formation in white clover roots and is regulated by flavonoids and derivatives of chitin oligosaccharides”, Plant J 14:23-34), induces periodic bumps that co-localize with elevated GH3:GUS activity (FIG. 3H) suggesting an alteration to auxin distribution at bump sites. Sectioning revealed intense GH3:GUS expression in bump vascular tissue. The histology of bumps housing arrested lateral roots confirmed their poor connection to root vasculature (FIG. 3L, 3M); the arrested lateral roots lacked GH3:GUS expression, although a patch of GH3:GUS expression was observed ahead of some arrested lateral roots (FIG. 3M).

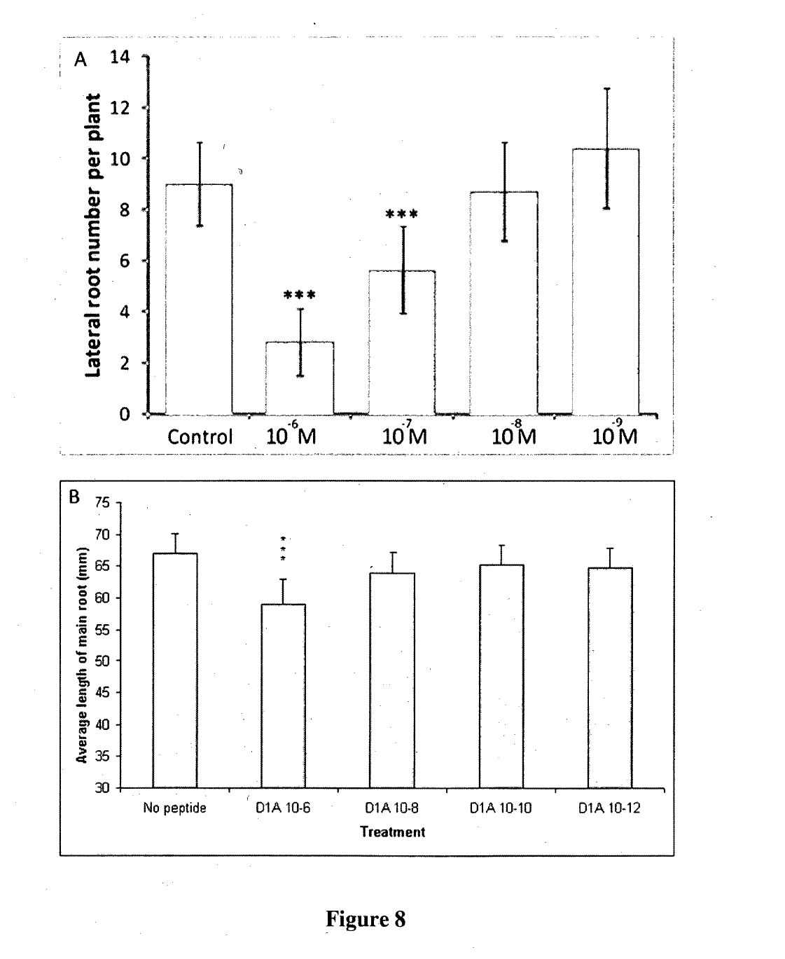

Biological activity of RAR ligands was confirmed using two synthetic M. truncatula MtRAR1 ligands and a M. hapla RAR2 ligand (FIGS. 1 and 6 vs 5D), and SEQ ID NOs 166 and 167—MtRAR1 domains—and 398—MhRAR2 domain) each made with the expected post-translational modifications. Wild-type plant roots exposed to these peptides at or above 10⁻⁷ M phenocopied MtRAR1ox roots by showing periodic bump (CCP site) formation, perturbation of root hair development (FIGS. 3D-E) and an inhibition of lateral root formation (FIG. 8A). In some instances, primary root growth was also inhibited (FIG. 8B). Confocal microscopy showed that CCP sites were 80 μm wider than a non-bump (CCP) site (FIG. 4E) which was attributable to cortical, epidermal and pericycle cell divisions. A more than two-fold increase in cortical cell number occurred at the bump sites and these cells possessed central nuclei (P<0.0001; FIGS. 4C and F). Arrested lateral roots contained small undifferentiated cells (FIG. 4D).

Inhibition of lateral root emergence was sustained over a two month period of growth by the addition to roots of the regulatory peptides: The inhibition of lateral root emergence by RAR1 peptide occurred in a dose-dependent manner at greater than 10⁻⁷ M, and was not affected by inoculation by S. meliloti. Because several residues in the 15 amino acid RAR domain were highly conserved in dicots, peptide variants were synthesised and tested for biological activity. These included peptides which were otherwise identical to MtRAR1 but which included deletion of the highly conserved C-terminal histidine, or substitution of the highly conserved glycine at position 8, or removal of the N-terminal residue or sequential removal of the proline post-translational modifications at positions 4 and 11. The PGN sequence of the MtRAR1 peptide is indicative of a beta turn which would be an important structural feature of the mature peptide in vivo, and substitution of Gly-8 would affect this structural feature. Structure activity relationship studies conducted with these MtRAR1 peptide variants showed that inhibition of lateral root emergence was abolished by removing the conserved C-terminal histidine residue or greatly attenuated by substituting the strongly conserved glycine at position eight to alanine, removing the N-terminal residue, or by not hydroxylating the proline residues.

The studies disclosed herein indicate that MtRAR1 regulates the developmental pathways of lateral roots, nodules and root hairs. Lateral root emergence is significantly reduced by over-expressing MtRAR1 and instead periodic bumps form, some of which house arrested lateral roots. The initiation of periodic bumps occurs in the zone of elongation and their rapid formation over ten hours suggests that they may coincide with the lateral root ‘pre-branch sites’ identified in Arabidopsis (Moreno-Risueno M A, et al. (2010), “Oscillating Gene Expression Determines Competence for Periodic Arabidopsis Root Branching”, Science 329:1306-1311). Alternatively RAR may inhibit lateral root formation after pre branch site formation. When arrested lateral roots occur at CCP sites (about 20% of CCP sites) they lack cellular differentiation and fail to attract vascular connections, indicating that these organs are locked at a developmental checkpoint. CCP sites that lack arrested lateral roots exhibit anticlinal cell divisions of cortical, epidermal and pericycle cells, and an extra cortical cell layer is observed which presumably arises by periclinal division. The increased number of epidermal cells at bumps could account for the increased root hair development.

Increased root nodule formation occurred on plants over-expressing. MtRAR1. This may indicate that bumps are more susceptible to rhizobial infection. Bumps partially resemble an early root nodule primordium, which initially comprises a focus of anticlinal cell divisions in the cortex, endodermis and pericycle. Rhizobia are known to ‘hijack’ sites of endogenous cell division sites in legumes, and this observation strengthens the developmental link between nodule ontogeny and gall formation.

### Example 6

### Nodulation is Enhanced by Ectopic Expression of MtRAR1 or by Peptide Application

The effect of over-expressing MtRAR1 on nodule organogenesis under different nitrogen regimes was studied.

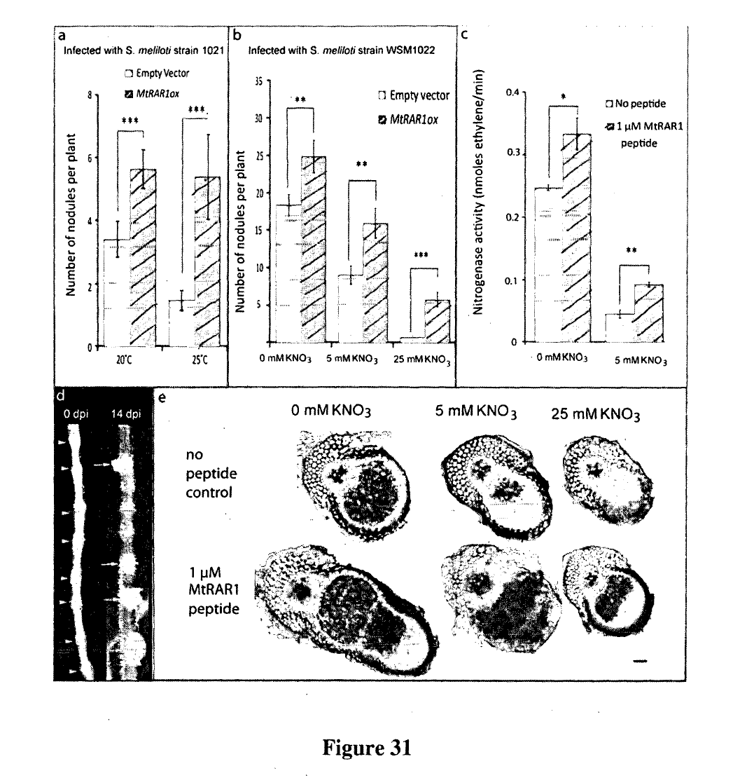

The data shown in FIG. 31 relates to studies that show that MtRAR/over-expression increases nodulation and promotes nodule development at different nitrate concentrations: (a) Comparison of nodulation between MtRAR1 over-expressing plants and control plants at different temperatures when inoculated with S. meliloti strain 1021. (b) Partial nitrate tolerance of nodule development induced by WSM1022 on MtRAR1 over-expressing plants. Three weeks old composite plants were inoculated and scored two weeks post inoculation. N≧30. student's t-test; *: P<0.05; **: P<0.01; ***: P<0.001. (c) Acetylene reduction assay. MtRAR1 treated (1 μM) and non treated wild-type plants grown in Fåhraeus medium were harvested at 2 week post inoculation by S. meliloti WSM1022. Nitrogenase activity was calculated from peak areas of samples relative to acetylene and ethylene standards to obtain moles ethylene/min. N≧3. student's t-test; *: P<0.05; **: P<0.01. Error bars indicate SE. (d) Inoculation of plants with preformed CCPs led to root nodule formation at CCP sites at 25 mM KNO₃. Arrow head: CCP sites; Arrows: nodules. (e) Nodule sections showing accelerated nodule development in RAR1 peptide-treated compared to untreated plants grown at either 0, 5 mM or 25 mM KNO₃ for three weeks. Scale bar: 100 μm. Plants were grown at 20° C.

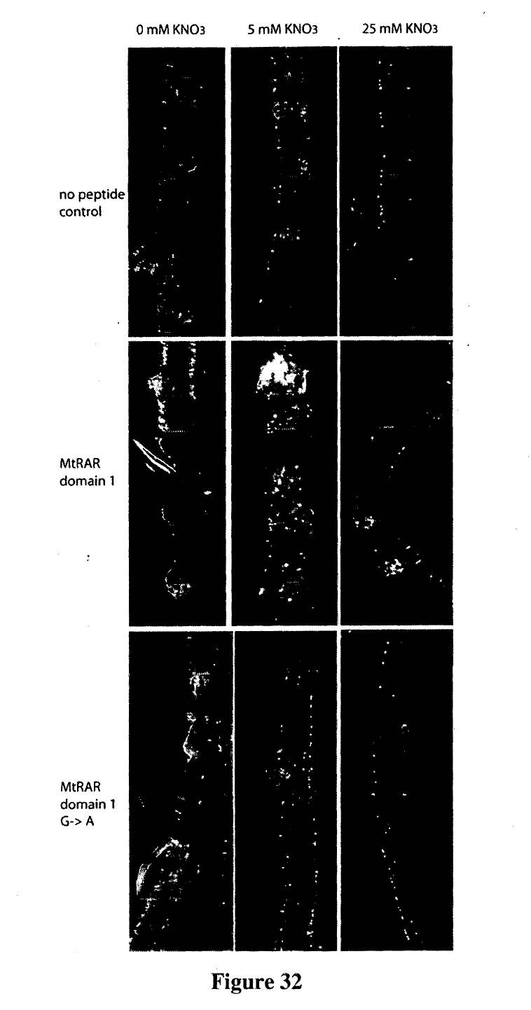

FIG. 32: M. truncatula plants were grown in 0.8% agar-containing plates supplemented with F media with or without MtRAR1 peptide. Five-day old seedlings were inoculated with S. meliloti strain WSM1022 and further grown for two more weeks. MtRAR1 Domain 1: AFQ(P)TTPGNS(P)GVGH; MtRAR1 Domain 1 with G->A transition: AFQ(P)TTPANS(P)GVGH. (P) is hydroxylproline and the altered amino acid is underlined.

The data shown/illustrated in FIGS. 31 and 32 indicates that more nodules formed on composite plants over-expressing MtRAR1 compared to controls when they were infected by S. meliloti and grown on either 20° C. or 25° C. in the absence of nitrogen (FIG. 31a). This extent of increased nodulation was more evident when the plants were inoculated with the model S. meliloti strain 1021 which induces poor nodule numbers with poorly symbiotic performance on the control plants compared to the plants inoculated with the highly effective strain WSM1022 which nodulates and fixes nitrogen more vigorously (FIG. 31a-b).

Because MtRAR1 peptide and MtRAR1 over-expression raised nodule number, the effect of MtRAR1 on symbiotic capacity, as measured by the frequency of pink nodules formed at 5 mM and 25 mM KNO₃ was also scored, as leghemoglobin production is an important indicator of symbiotic capacity. There were significantly higher numbers of pink functional nodules on plants over-expressing MtRAR1 or exposed to 1 μM MtRAR1 peptide compared to controls (FIG. 32). At 5 mM and 25 mM KNO₃, the nodule numbers formed on roots over-expressing MtRAR1 increased significantly (e.g. 10-fold higher at 25 mM nitrate; FIG. 31b). A similar trend was observed when 1 μM MtRAR1 peptide was applied to the plants but not when the non-functional MtRAR1 peptide where the central glycine residue was replaced with alanine was applied (FIG. 32). All across the nitrate regimen, the nodules were much larger on MtRAR1 peptide treated roots than on plants treated with no peptide or with the MtRAR1 peptide where the central glycine residue was replaced with alanine (FIGS. 31e and 32). After three weeks post inoculation with S. meliloti, a clear bacteroid like zone was apparent in nodule sections on MtRAR1 peptide-treated plants and nodules often spanned across two xylem poles; nodules at 25 mM nitrate possessed leghemoglobin (FIG. 31e). At 25 mM nitrate, nodules were induced at, but not between, preexisting CCP sites on plants over-expressing MtRAR1 or plants treated with peptides (FIG. 31d). The results showed that for plants exposed to elevated MtRAR1 by over-expression or ectopic application, the susceptibility to infection by S. meliloti and nodulation ability was more tolerant to nitrate and overall nodulation development was enhanced. To further confirm the enhancement of nodulation by RAR addition, we measured nitrogenase activity using acetylene reduction assay. The results showed significant increase in the nitrogenease activity from 35% at 0 mM KNO₃ to 200% at 5 mM KNO₃ when 1 μM MtRAR1 was applied (FIG. 31c).

### Example 7

### MtRAR1 Over-Expression Regulates Key Genes Involved in N-Uptake Pathways

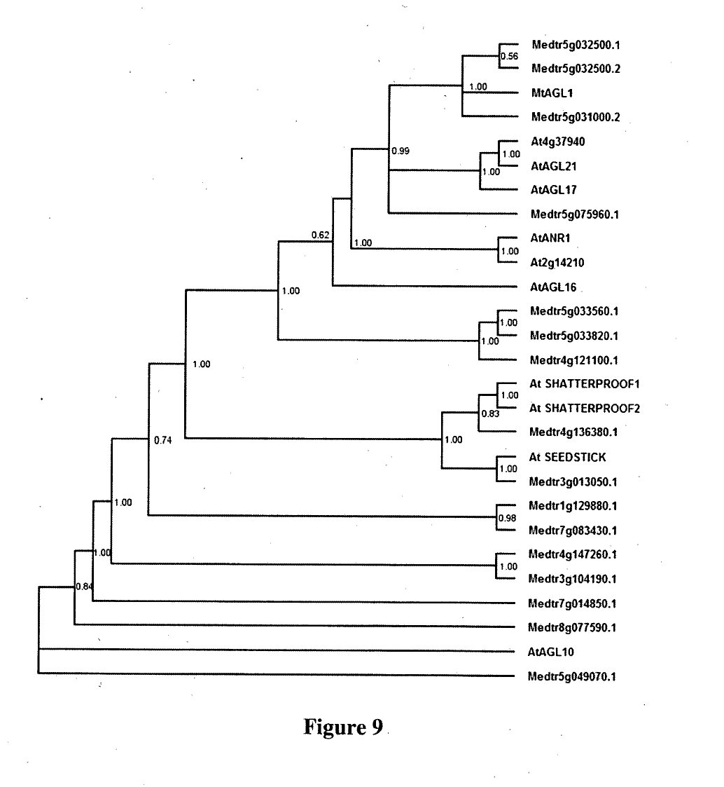

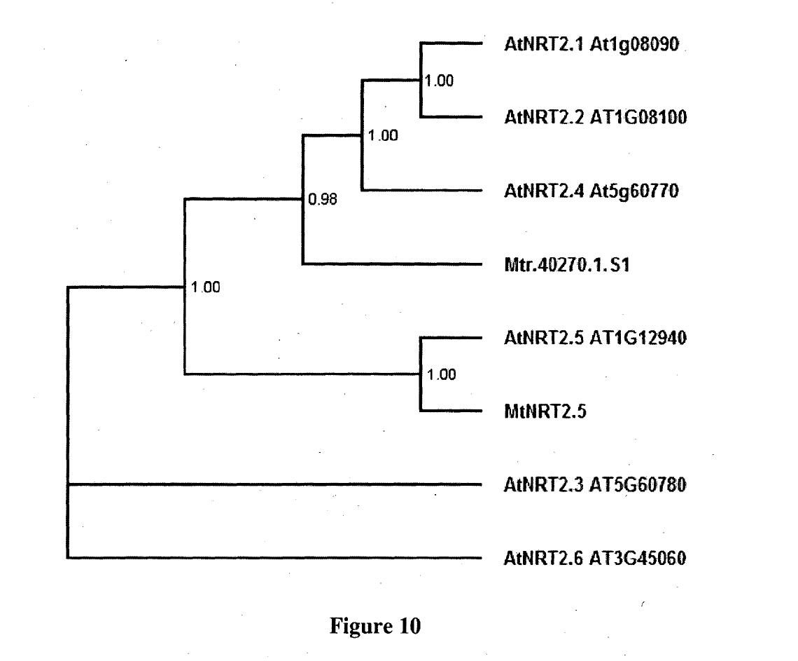

As its expression responded to N-status, we investigated the role of MtRAR1 in controlling M. truncatula homologs of key genes that regulate the N-signaling network in Arabidopsis. Arabidopsis MADS box transcription factors ANR1 and AGL21 (AGAMOUS-LIKE 21) are known to regulate lateral root growth in response to external nitrogen levels (Gan Y, Filleur S, Rahman A, Gotensparre S, & Forde B G (2005), “Nutritional regulation of ANR1 and other root-expressed MADS-box genes in Arabidopsis thaliana”, Planta 222:730-742). Over-expression of MtRAR1 up-regulates the ANR1/AGL21 homolog MtAGL1 (FIGS. 3J and 9) and down-regulates MtLBD38, a LATERAL ORGAN BOUNDARIES-domain containing transcription factor that negatively regulates N-availability signals (Rubin G, Tohge T, Matsuda F, Saito K, & Scheible W R (2009), “Members of the LBD family of transcription factors repress anthocyanin synthesis and affect additional nitrogen responses in Arabidopsis”, Plant Cell 21:3567-3584) and the high affinity nitrate transporter MtNRT2.5 (FIGS. 3J and 10).

### Example 8

### Exemplifications of RAR Peptide Effect in Lucerne, Subterranean Clover, White Clover, Tomato, Soybean and Rice Plants

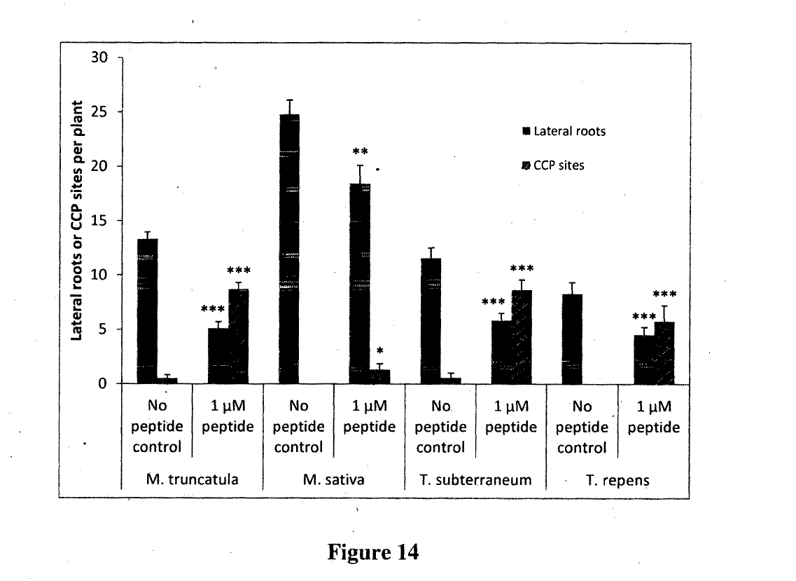

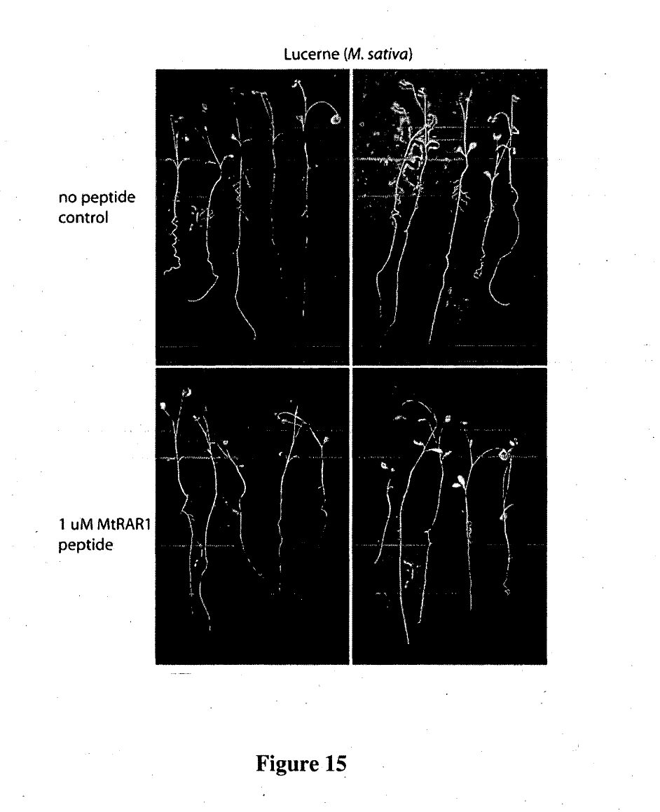

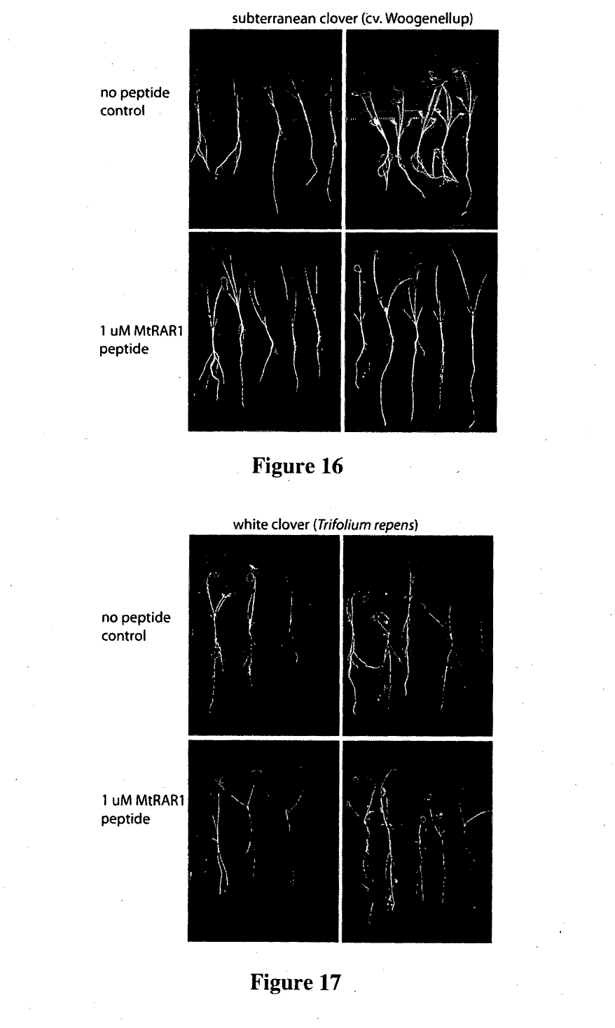

To further validate and exemplify the RAR technology, we have examined RAR peptide addition of roots of various plant species. Lateral root emergence was significantly inhibited (students' t-test P<0.05) by the addition to roots of the regulatory peptide 1 μM MtRAR1 in M. truncatula, Lucerne (M. sativa), Subterranean Clover (Cultivar Woogenellup) and white clover (Trifolium repens) as shown in FIGS. 14-17. These peptide treated legume plants not only showed altered lateral root phenotype similar to M. truncatula, they also formed CCP (circumferential cell proliferations; “bump”) sites similar to the ones in M. truncatula.

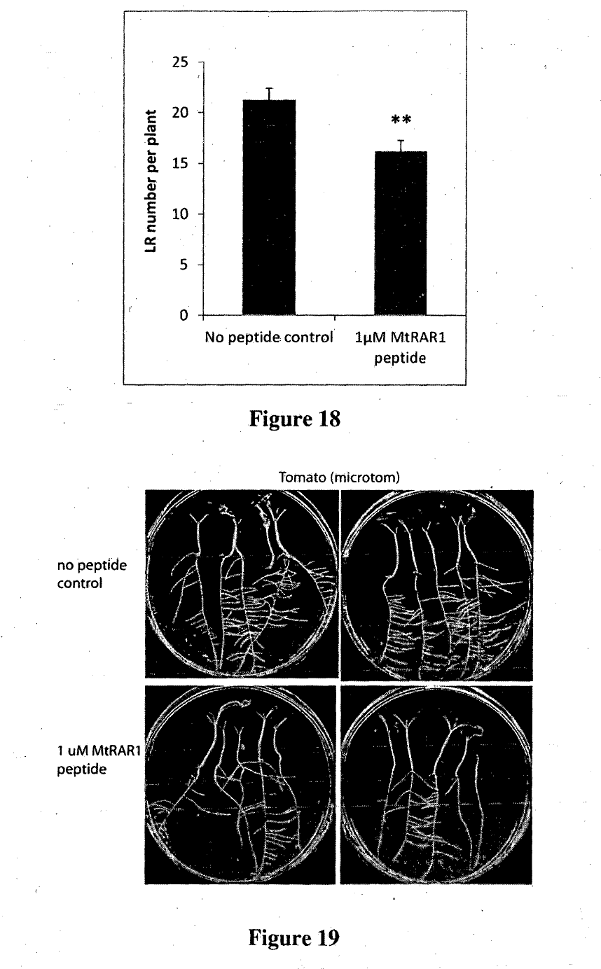

Using plate assays, we also have tested effect of 1 μM MtRAR1 peptides on microtom tomato seedlings and data showed significant (students' t-test P<0.05) reduction in number of lateral roots when 1 μM MtRAR1 was applied into the liquid medium (FIGS. 18 and 19). Primary root length was not affected (FIG. 6).

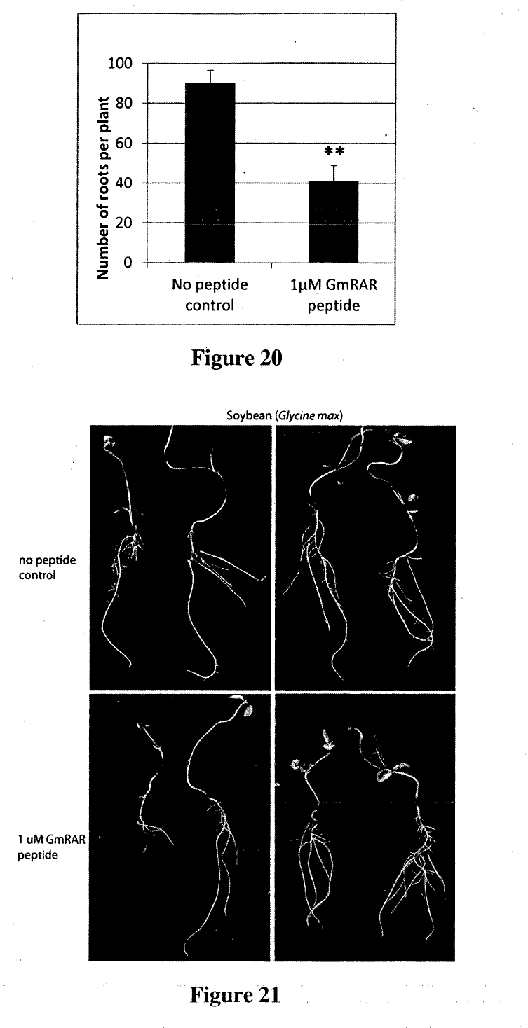

Using liquid culture system, we also have tested effect of dicot RAR peptides on soybean seedlings and data showed significant (students' t-test P<0.05) reduction in number of roots when 1 μM dicot RAR was applied into the liquid medium (FIGS. 20 and 21). Primary root length was also affected (FIG. 21).

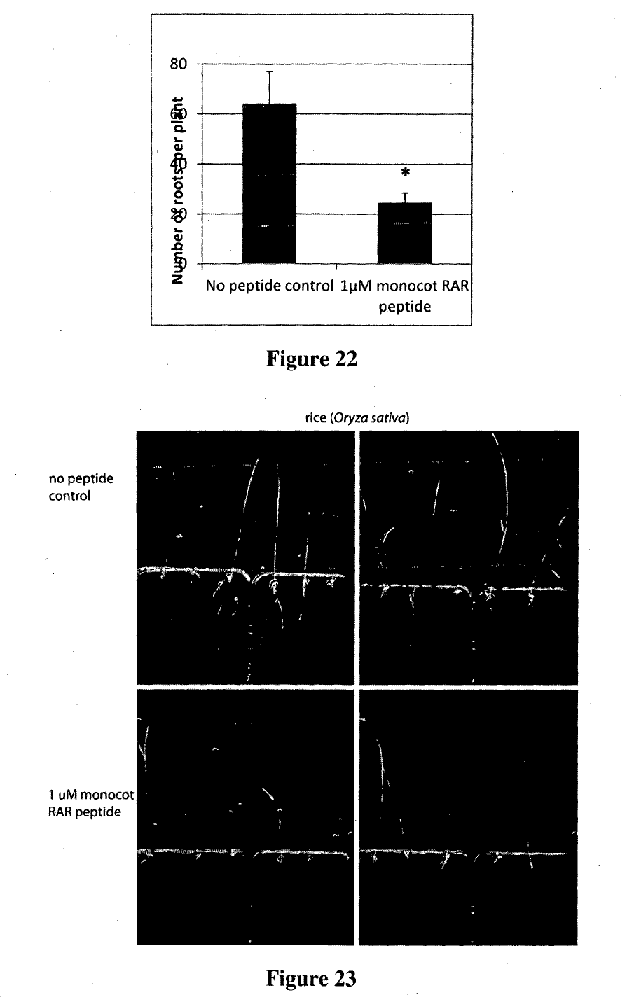

Using the same liquid culture system but supplemented with half stress MS medium, we have tested effect of monocot RAR peptides on rice seedlings and data showed significant (students' t-test P<0.05) reduction in number of roots when 1 μM dicot RAR was applied into the liquid medium (FIGS. 22 and 23). Primary root length was also affected (FIG. 23).

### Example 9

### Phenotypic Analysis: RAR Peptide Addition in Arabidopsis

To test the effect or RAR peptide addition on Arabidopsis thaliana roots, wild type (WT) ecotype Columbia (Col) plants were grown vertically on ½ MS with 1% phytagel and 1 μM of RAR peptide (see Table 3, overleaf) or no peptide, with 16 hr days at 22° C. Plates were imaged at 12 days and analysed with the SmartRoot plugin in ImageJ. Several different peptides with varied modifications were assayed. Modifications: hyP=2 hydroxylated proline residues, no hyP=no hydroxylated prolinate residues, phos=phosphorylation on residue 6 plus 2 hydroxylated proline residues.

As can be seen from the results, all RAR peptides tested have the same effect in Arabidopsis: primary root length is reduced and the total number of lateral roots per plant is reduced; however the extent of the phenotype differs.

TABLE 3
List of RAR peptides assayed and sequence.

| Peptide | Sequence |
|---|---|
| RAR3 hyP | D T F R hyP T E P G H S hyP G I G H |
| RAR5 hyP | D F V hyP T S P G N S hyP G V G H |
| (domain 1) | |
| RAR5 no HyP | D F V P T S P G N S P G V G H |
| (domain 1) | |
| RAR5 phos | D F V hyP T (p-S) P G N S hyP G V G H |
| (domain 1) | |
| RAR6 hyP | D F R hyP T T P G H S hyP G I G H |
| RAR6 phos | D F R hyP T (p-T) P G H S hyP G I G H |

hyP indicates hydroxyproline residues.
(p-) indicates a phosphorylated residue.

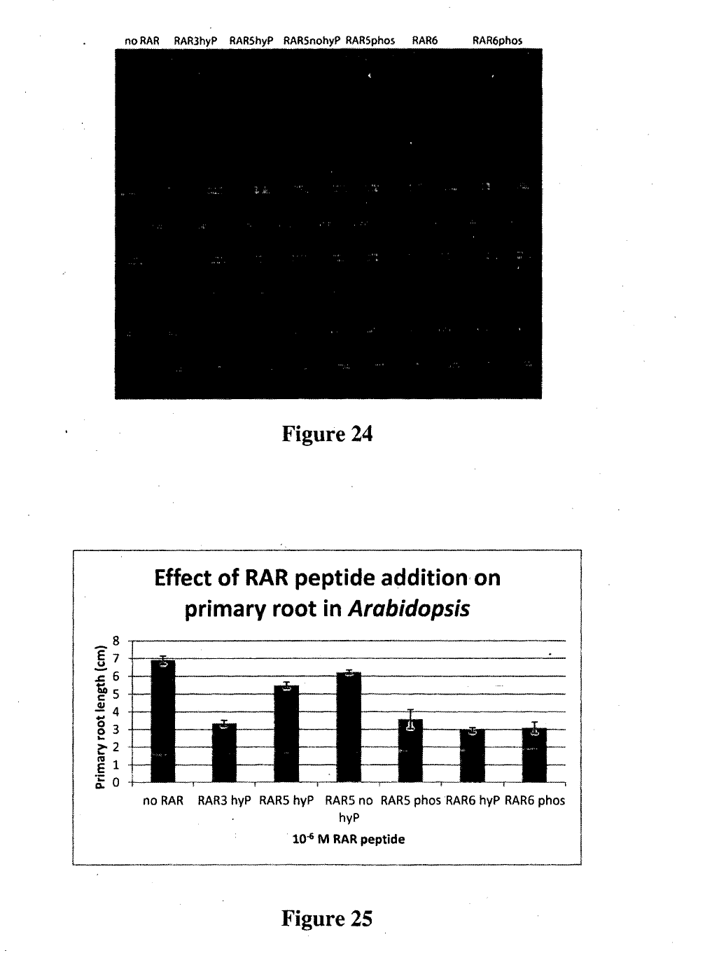

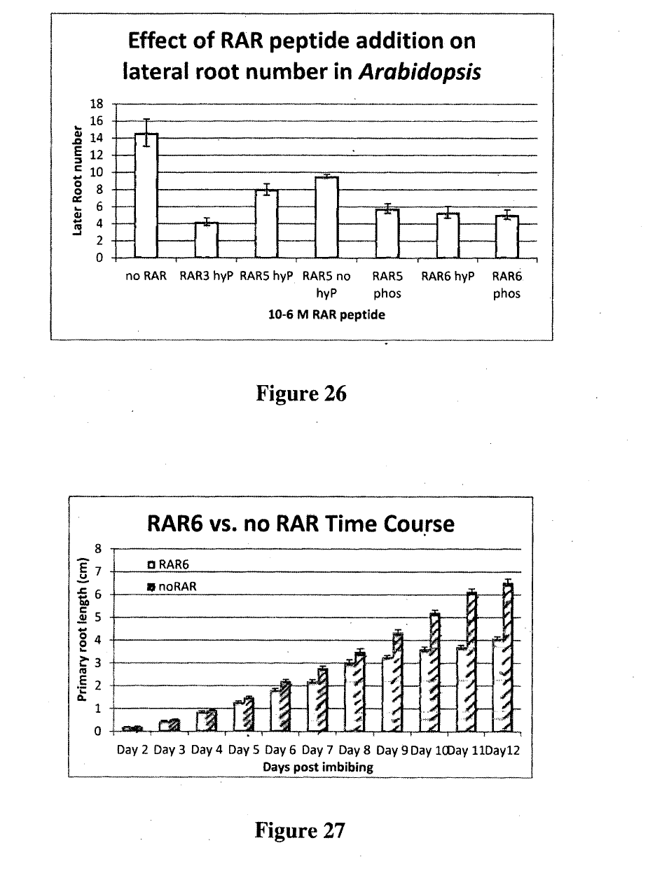

The results are shown in FIGS. 24 to 26. In FIG. 24, scale bar=1 cm. In FIGS. 25 and 26, error bars are +/−standard error. Significance:* p<0.05; ** p<0.005; ***p<0.001.

### Example 10

### RAR Bioactivity in Arabidopsis

To investigate the effect of different modifications on peptide bioactivity, several peptides were assayed for activity at different concentrations (Table 4). WT Col plants were grown vertically on ½ MS plates with 1% phytagel and containing varying concentration of RAR peptide ranging from 10⁻⁶ M to 10⁻¹² M or no peptide. Plates were imaged at 12 days.

TABLE 4
Bioactivity of different peptides at varied concentrations.

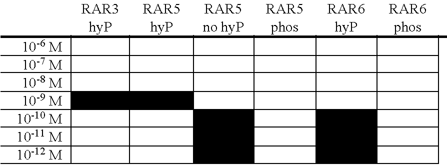

Modifications:
hyP = 2 hydroxylated praline residues,
no hyP = no hydroxylated prolinate residues,
phos = phosphorylation on residue 6 plus 2 hydroxylated proline residues.
light shading indicates biological activity (ie. primary root length and number of lateral roots are significantly lower than untreated samples, p < 0.05).
Dark shading indicates no biological activity (treated plants not significantly different to untreated samples, p > 0.05).
No shading indicates the concentration was not assayed.

The most bioactive peptide is phosphorylated RAR6, which is active at picomolar concentrations. It is 1000× more active than the non-phosphorylated RAR6 peptide. The RAR5 peptide appears to be more active when it is not hydroxyprolinated compared to a hydroxylated sample. It is active at a 10⁻⁹ M compared to the hydroxyprolinated peptide, which is active at 10⁻⁸ M. However phosphorylation of Arabidopsis RAR5 enhances activity.

In conclusion, different modifications, including hydroxylation of two proline residues and phosphorylation affect peptide bioactivity in Arabidopsis.

### Example 1.1

### Time Course: RAR Action in Arabidopsis Over Time

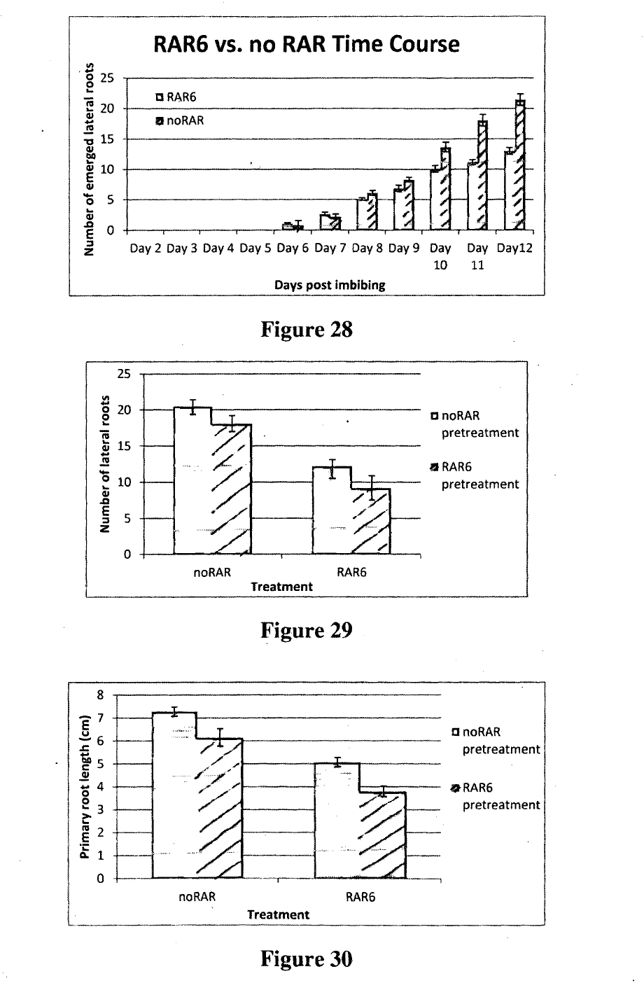

To see how RAR was exerting its effect on PR growth and lateral root emergence and growth, a time course was performed over 12 days. WT Col plants were grown vertically on ½ MS with 1% phytagel and 1 μM RAR6 hyP or no peptide, with 16 hr days at 22° C. Plates were scanned every day for 12 days. The results are shown in FIGS. 27 and 28.

In FIG. 27 it can be seen that the primary root length was significantly (p<0.05) different on all days except day 2 (marked ns=not significant). Error bars are +/−standard error.

In FIG. 28 it can be seen that the number of lateral roots was not significantly different until day 8. Error bars are +/−standard error. * p<0.05; p<0.005; ***p<0.001.

Overall the data show that germination and root establishment are not affected by RAR treatment as there was no significant difference in PR length between treated and untreated samples at 2 days post imbibing. However, from day 3 onwards, the PR length in RAR treated plants was significantly shorter than in untreated plants. The difference in lateral root number was not significant between treated and untreated samples until day 8. This was 2 days after the first lateral roots appeared in both treatments. Therefore, the emergence of the first lateral roots are not affected by RAR, however subsequent lateral root emergence is affected.

### Example 12

### Rescue: Removing RAR Releases its Effect

To investigate whether the effect of RAR peptide addition could be released, WT Col plants were grown vertically for 6 days on ½ MS plates with 1% phytagel. Plants were transferred from plates containing no RAR or 1 μM RAR6 hyP peptide (pre-treatment) to plates containing no RAR or 1 μM RAR6 hyP peptide as specified where they were left to grow for a further 6 days before being analysed.

The results are illustrated in FIGS. 29 and 30. FIG. 29 shows that the number of lateral roots is the same in plants transferred to the no RAR treatment regardless of pre-treatment (p<0.05), and FIG. 30 shows that the primary root length of plants pre-treated with RAR, and transferred to no RAR is significantly higher (p<0.05) than plants exposed to RAR continuously. Error bars are +/−standard error. Letters indicate samples that are significantly different from each other (p<0.05).

The data show that the number of lateral roots is the same in plants transferred to the no RAR treatment regardless of pre-treatment. It can therefore be concluded that the effect of RAR peptide addition on the total number of lateral roots can be released by removing RAR exposure. This is further supported by the lack of significant difference in the number of lateral roots in plants transferred to RAR6 plates from either RAR6 or no RAR pre-treatments. Additionally, primary root length of plants pre-treated with RAR and transferred to no RAR is significantly higher than plants exposed to RAR continuously, indicating that the plants begin to recover upon removal of RAR.

The results described herein indicate that RARs mediate root development in response to nutritional cues, a process that evolved most intricately in the plants with true roots. These observations may be used to affect plant root architecture to, for example, allow plants to more efficiently utilise nutrient resources, or to grow in environments that wild-type counterparts typically do not.

The present studies also show that RARs are unique regulators in higher plants and the obligate parasites that subvert their innate developmental processes. This suggests that RKNs have targeted an indispensible system in higher plants required for true root development. These observations not only provide insight into how RKNs interact with plants, but also point to mechanisms for interfering with this interaction and to inhibit or lessen the significant damage to crop plants often caused by RKNs.

It will be appreciated that, although specific embodiments of the invention have been described herein for the purpose of illustration, various modifications may be made without deviating from the spirit and scope of the invention as defined in the following claims.

1. A method for modulating or inhibiting modulation of the root architecture of a plant, said method comprising:
(a) contacting the root zone of said plant with a root architecture regulator peptide (RAR), an analogue thereof or an RAR signaling agonist; or
(b) contacting the root zone of said plant with a RAR signaling antagonist; or
(c) introducing at least one mutation or exogenous nucleic acid into one or more plant cells which results in modulated RAR expression by a root knot nematode or by cells of a plant regenerated from or comprising said one or more plant cells; or
(d) introducing at least one mutation or exogenous nucleic acid into one or more plant cells which results in modulated RAR receptor expression by cells of a plant regenerated from or comprising said one or more plant cells; or
(e) introducing at least one mutation or exogenous nucleic acid into one or more plant cells which results in modulated affinity of one or more RARs for their respective RAR receptors, which modulated affinity arises through modifications in the RAR(s), RAR receptor(s) or in both expressed RAR(s) and RAR receptor(s); or
(f) introducing at least one exogenous nucleic acid into one or more plant cells which results in expression of one or more root knot nematode RAR binding agents or antagonists.

2. The method of claim 1, for reducing lateral root development by a plant relative to an untreated or wild-type plant, comprising contacting the root zone of said plant with at least one RAR or introducing at least one exogenous RAR-encoding nucleic acid into one or more plant cells which results in increased RAR expression by cells of a plant regenerated from or comprising said one or more plant cells.

3. The method of claim 2, wherein said RAR-encoding nucleic acid is operably linked to a promoter.

4. The method of claim 3, wherein said promoter is constitutive or inducible by nutrient status.

5. The method of claim 4, wherein said promoter is inducible by nitrogen starvation or high carbon dioxide.

6. The method of claim 3, wherein said promoter is root-specific.

7. The method of claim 3, wherein said promoter is the Medicago truncatula RAR1 promoter (SEQ ID NO: 337)

8. The method of claim 2, wherein said plant comprises a greater density of root hairs at periodic root bumps.

9. The method of claim 2, wherein said RAR is not an Arabidopsis CEP1 peptide and said RAR-encoding nucleic acid is not a Arabidopsis CEP1-encoding sequence.

10. The method of claim 2, wherein said RAR comprises an amino acid sequence selected from SEQ ID NOs: 4, 6, 8, 10, 12, 14, 16, 18, 20, 22, 24, 26, 28, 30, 32, 34, 36 to 147, 338-350, 365, 367, 369, 371, 373, 375, 377, 379, 381, 383, 385, 387-395 or comprises an RAR domain having an amino acid sequence selected from SEQ ID Nos: 148-336, 351-363 or 396-415, or wherein said RAR-encoding nucleic acid comprises a nucleotide sequence selected from SEQ ID NOs 3, 5, 7, 9, 11, 13, 15, 17, 19, 21, 23, 25, 27, 29, 31, 33, 35, 364, 366, 368, 370, 372, 374, 376, 378, 380, 382, 384 or 386.

11. The method of claim 2, wherein said RAR is a root knot nematode RAR.

12. The method of claim 11, wherein said RAR comprises an amino acid sequence selected from SEQ ID NOs 365, 367, 369, 371, 373, 375, 377, 379, 381, 383, 385, 387-395 or comprises an RAR domain having an amino acid sequence selected from SEQ ID Nos: 396-415 or said RAR-encoding nucleic acid comprises a nucleotide sequence selected from SEQ ID NOs: 364, 366, 368, 370, 372, 374, 376, 378, 380, 382, 384 or 386.

13. The method of claim 2, wherein said plant is selected from members of the angiosperm families Aceraceae, Anacardiaceae, Apiaceae, Asteraceae, Betulaceae, Brassicaceae, Buxaceae, Chenopodiaceae/Amaranthaceae, Compositae, Cucurbitaceae, Fabaceae, Fagaceae, Gramineae, Juglandaceae, Lamiaceae, Lauraceae, Leguininosae, Moraceae, Myrtaceae, Oleaceae, Platanaceae, Poaceae, Polygonaceae, Rosaceae, Rutaceae, Salicaceae, Solanaceae, Ulmaceae or Vitaceae or gymnosperm families Cuppressaceae, Pinaceae, Taxaceae or Taxodiaceae.

14. The method of claim 2 which results in, or results in an increase in symbiotic nitrogen-fixing nodule numbers on the root(s) of said plant.

15. The method of claim 14, wherein said plant is a member of the Fabaceae or the Leguminoseae.

16. The method of claim 1, for modulating lateral root growth of a plant relative to an untreated or wild-type plant, comprising contacting the root zone of said plant with a RAR antagonist or introducing at least one mutation or at least one exogenous nucleic acid into one or more plant cells which at least one mutation or nucleic acid results in:
(i) decreased expression of one or more RARs, decreased expression of one or more RAR receptors, or decreased expression of one or more RARs and one or more RAR receptors by cells of a plant regenerated from or comprising said one or more plant cells, wherein said decreased expression of said RAR(s) or RAR receptor(s) occurs under conditions which would otherwise promote expression of said RAR(s) or RAR receptor(s); or
(ii) reduced affinity of one or more RARs for their respective RAR receptors, which reduced affinity arises through modifications in the RAR(s), RAR receptor(s) or in both expressed RAR(s) and RAR receptor(s) expressed by cells of a plant regenerated from or comprising said one or more plant cells.

17. The method of claim 16, which comprises introducing into said one or more plant cells exogenous nucleic acid which suppresses, or the product of which suppresses expression of at least one endogenous RAR-encoding nucleic acid.

18. The method of claim 17, wherein said exogenous nucleic acid comprises a nucleic acid sequence homologous to, or complementary to at least a portion of the endogenous RAR-encoding nucleic acid.

19. The method of claim 17, wherein said exogenous nucleic acid is, or encodes a microRNA or siRNA.

20. The method of claim 16 wherein said plant is selected from members of the angiosperm families Aceraceae, Anacardiaceae, Apiaceae, Asteraceae, Betulaceae, Brassicaceae, Buxaceae, Chenopodiaceae/Amaranthaceae, Compositae, Cucurbitaceae, Fabaceae, Fagaceae, Gramineae, Juglandaceae, Lamiaceae, Lauraceae, Leguminosae, Moraceae, Myrtaceae, Oleaceae, Platanaceae, Poaceae, Polygonaceae, Rosaceae, Rutaceae, Salicaceae, Solanaceae, Ulmaceae or Vitaceae or gymnosperm families Cuppressaceae, Pinaceae, Taxaceae or Taxodiaceae.

21. The method of claim 1 for inhibiting modulation of the root architecture of a plant by the action of a root knot nematode.

22. The method of claim 21, which comprises contacting the root zone of said plant with a root knot nematode RAR antagonist or binding agent.

23. The method of claim 21, wherein said root knot nematode antagonist comprises a root knot nematode RAR binding agent.

24. The method of claim 21, which comprises introducing into said one or more plant cells at least one exogenous nucleic acid which suppresses, or the product of which suppresses expression of a root knot nematode RAR-encoding nucleic acid.

25. The method of claim 24, wherein said exogenous nucleic acid comprises or encodes a nucleic acid sequence homologous to, or complementary to at least a portion of a root knot nematode RAR-encoding nucleic acid.

26. The method of claim 25, wherein said root knot nematode RAR-encoding nucleic acid comprises a nucleotide sequence selected from SEQ ID NOs: 364, 366, 368, 370, 372, 374, 376, 378, 380, 382, 384 or 386, or encodes a peptide comprising an amino acid sequence selected from SEQ ID NOs: 365, 367, 369, 371, 373, 375, 377, 379, 381, 383, 385, 387-415.

27. The method of claim 25, wherein said exogenous nucleic acid is, or encodes a microRNA or siRNA.

28. The method of claim 21, which comprises introducing into said one or more plant cells one or more exogenous nucleic acids which code for a root knot nematode RAR binding agent or antagonist and secretion thereof.

29. The method of claim 28, wherein said binding agent is an antibody.

30. The method of claim 28, wherein said root knot nematode RAR comprises an amino acid sequence selected from SEQ ID NOs: 365, 367, 369, 371, 373, 375, 377, 379, 381, 383, 385, 387-395 or comprises, an RAR domain having an amino acid sequence selected from SEQ ID Nos: 396-415.

31. The method of claim 21, wherein said plant is selected from members of the angiosperm families Aceraceae, Anacardiaceae, Apiaceae, Asteraceae, Betulaceae, Brassicaceae, Buxaceae, Chenopodiaceae/Amaranthaceae, Compositae, Cucurbitaceae, Fabaceae, Fagaceae, Gramineae, Juglandaceae, Lamiaceae, Lauraceae, Leguminosae, Moraceae, Myrtaceae, Oleaceae, Platanaceae, Poaceae, Polygonaceae, Rosaceae, Rutaceae, Salicaceae, Solanaceae, Ulmaceae or Vitaceae or gymnosperm families Cuppressaceae, Pinaceae, Taxaceae or Taxodiaceae.

32. A plant with modulated root architecture relative to an untreated or wild-type plant, obtained by the method of claim 1, or a part thereof.

33. A plant with reduced lateral root development relative to an untreated or wild-type plant, obtained by the method of claim 2.

34. A plant with increased lateral root development relative to an untreated or wild-type plant, obtained by the method of claim 15.

35. A plant with increased resistance to root architecture modulation by root knot nematodes relative to an untreated or wild-type plant, obtained by the method of claim 22.

36. The plant of any one of claims 32 to 35, wherein said plant is selected from members of the angiosperm families Aceraceae, Anacardiaceae, Apiaceae, Asteraceae, Betulaceae, Brassicaceae, Buxaceae, Chenopodiaceae/Amaranthaceae, Compositae, Cucurbitaceae, Fabaceae, Fagaceae, Gramineae, Juglandaceae, Lamiaceae, Lauraceae, Leguminosae, Moraceae, Myrtaceae, Oleaceae, Platanaceae, Poaceae, Polygonaceae, Rosaceae, Rutaceae, Salicaceae, Solanaceae, Ulmaceae or Vitaceae or gymnosperm families Cuppressaceae, Pinaceae, Taxaceae or Taxodiaceae.

37. An isolated root architecture regulator peptide (RAR) or an analogue thereof, with the proviso that said RAR is not an Arabidopsis CEP1 peptide and said RAR-encoding nucleic acid is not a Arabidopsis CEP1-encoding sequence.

38. The peptide of claim 37, wherein said RAR comprises an amino acid sequence selected from SEQ ID NOs 4, 6, 8, 10, 12, 14, 16, 18, 20, 22, 24, 26, 28, 30, 32, 34, 36 to 147, 338-350, 365, 367, 369, 371, 373, 375, 377, 379, 381, 383, 385, 387-395 or comprises an RAR domain having an amino acid sequence selected from SEQ ID Nos: 148-336, 351-363 or 396-415, or is encoded by a nucleic acid comprising a nucleotide sequence selected from SEQ ID NOs 3, 5, 7, 9, 11, 13, 15, 17, 19, 21, 23, 25, 27, 29, 31, 33, 35, 364, 366, 368, 370, 372, 374, 376, 378, 380, 382, 384 or 386.

39. The peptide of claim 37, wherein said RAR is a root knot nematode RAR.

40. The peptide of claim 39, wherein said RAR comprises an amino acid sequence selected from SEQ ID NOs 365, 367, 369, 371, 373, 375, 377, 379, 381, 383, 385, 387-395 or comprises an RAR domain having an amino acid sequence selected from SEQ ID Nos: 396-415 or is encoded by a nucleic acid comprising a nucleotide sequence selected from SEQ ID NOs 364, 366, 368, 370, 372, 374, 376, 378, 380, 382, 384 or 386.

41. An isolated polynucleotide comprising a nucleotide sequence coding for a RAR, with the proviso that said polynucleotide is not the Arabidopsis CEP1-encoding sequence.

42. The polynucleotide of claim 41, which also comprises a secretion signal peptide encoding sequence.

43. The polynucleotide of claim 41 which encodes a RAR comprising an amino acid sequence selected from SEQ ID NOs 4, 6, 8, 10, 12, 14, 16, 18, 20, 22, 24, 26, 28, 30, 32, 34, 36 to 147, 338-350, 365, 367, 369, 371, 373, 375, 377, 379, 381, 383, 385, 387-395 or comprises an RAR domain having an amino acid sequence selected from SEQ ID Nos: 148-336, 351-363 or 396-415, or which comprises a nucleotide sequence selected from SEQ ID NOs 3, 5, 7, 9, 11, 13, 15, 17, 19, 21, 23, 25, 27, 29, 31, 33, 35, 364, 366, 368, 370, 372, 374, 376, 378, 380, 382, 384 or 386.

44. The polynucleotide of claim 41, which encodes a root knot nematode RAR.

45. The polynucleotide of claim 43, wherein said RAR comprises an amino acid sequence selected from SEQ ID NOs 365, 367, 369, 371, 373, 375, 377, 379, 381, 383, 385, 387-395 or comprises an RAR domain having an amino acid sequence selected from SEQ ID Nos: 396-415 or is encoded by a nucleic acid comprising a nucleotide sequence selected from SEQ ID NOs 364, 366, 368, 370, 372, 374, 376, 378, 380, 382, 384 or 386.

46. A recombinant vector comprising the polynucleotide of claim 41.

47. A recombinant vector comprising the polynucleotide of claim 44.

48. A transgenic host cell comprising the polynucleotide of claim 41.

49. A transgenic host cell comprising the polynucleotide of claim 44.

50. The transgenic cell of claim 48 which is a plant cell.

51. The transgenic cell of claim 49 which is a plant cell.

52. A transgenic plant comprising the polynucleotide of claim 41.

53. A transgenic plant comprising the polynucleotide of claim 44.

54. A method for inhibiting modulation of the root architecture of a plant by the action of a root knot nematode, said method comprising:
(a) contacting the root zone of said plant with a root knot nematode RGF signaling antagonist;
(b) introducing at least one exogenous nucleic acid into one or more plant cells which results in suppression of expression of a root knot nematode RGF;
(c) introducing at least one mutation or exogenous nucleic acid into one or more plant cells which results in modulated affinity of one or more root knot nematode RGFs for plant RGF receptor(s), which modulated affinity arises through modifications in the plant RGF receptor(s); or
(d) introducing at least one exogenous nucleic acid into one or more plant cells which results in expression by said cell(s) of one or more root knot nematode RGF binding agents or antagonists.

55. The method of claim 54, which comprises contacting the root zone of said plant with a root knot nematode RGF antagonist or binding agent.

56. The method of claim 54, wherein said root knot nematode antagonist comprises a root knot nematode RGF binding agent.

57. The method of claim 54, which comprises introducing into said one or more plant cells at least one exogenous nucleic acid which suppresses, or the product of which suppresses expression of a root knot nematode RGF-encoding nucleic acid.

58. The method of claim 57, wherein said exogenous nucleic acid comprises or encodes a nucleic acid sequence homologous to, or complementary to at least a portion of a root knot nematode RGF-encoding nucleic acid.

59. The method of claim 58, wherein said root knot nematode RAR encodes a peptide comprising an amino acid sequence selected from SEQ ID NOs: 416 or 417 or comprises an RGF domain having an amino acid sequence selected from SEQ ID Nos: 460 or 461.

60. The method of claim 58, wherein said exogenous nucleic acid is, or encodes a microRNA or siRNA.

61. The method of claim 54, which comprises introducing into said one or more plant cells one or more exogenous nucleic acids which code for a root knot nematode RGF binding agent or antagonist.

62. The method of claim 61, wherein said binding agent is an antibody.

63. The method of claim 61, wherein said root knot nematode RAR comprises an amino acid sequence selected from SEQ ID NOs: 416 or 417 or comprises an RGF domain having an amino acid sequence selected from SEQ ID Nos: 460 or 461.

64. The method of claim 54, wherein said plant is selected from members of the angiosperm families Aceraceae, Anacardiaceae, Apiaceae, Asteraceae, Betulaceae, Brassicaceae, Buxaceae, Chenopodiaceae/Amaranthaceae, Compositae, Cucurbitaceae, Fabaceae, Fagaceae, Gramineae, Juglandaceae, Lamiaceae, Lauraceae, Leguminosae, Moraceae, Myrtaceae, Oleaceae, Platanaceae, Poaceae, Polygonaceae, Rosaceae, Rutaceae, Salicaceae, Solanaceae, Ulmaceae or Vitaceae or gymnosperm families Cuppressaceae, Pinaceae, Taxaceae or Taxodiaceae.

65. A plant with increased resistance to modulation of root architecture by the action of a root knot nematode, relative to an untreated or wild-type plant, obtained by the method of claim 54, or a part thereof.

66. An isolated polynucleotide comprising a nucleotide sequence coding for a root knot nematode RGF, or a portion thereof.

67. The polynucleotide of claim 66 which encodes a root knot nematode RGF comprising an amino acid sequence as shown in SEQ ID NO. 416 or 417 or comprises an RGF domain having an amino acid sequence selected from SEQ ID Nos: 460 or 461.

68. A recombinant vector comprising the polynucleotide of claim 66 or claim 67.

69. A transgenic host cell comprising the polynucleotide of claim 66 or claim 67.

70. The transgenic cell of claim 69 which is a plant cell.

71. A transgenic plant comprising the polynucleotide of claim 70.

72. A method for inhibiting modulation of the root architecture of a plant by the action of a root knot nematode, said method comprising introducing at least one exogenous nucleic acid into one or more plant cells which suppresses, or the product of which suppresses expression of a component of a root knot nematode signal peptidase complex.

73. The method of claim 72, wherein said component is signal peptidase I.

74. The method of claim 73, wherein said signal peptidase I comprises a nucleotide sequence as shown in SEQ ID NO: 419, or an amino acid sequence as shown in SEQ ID NO. 420.

75. The method of claim 72, wherein said exogenous nucleic acid comprises or encodes a nucleic acid sequence homologous to, or complementary to at least a portion of said component of a root knot nematode signal peptidase complex.

76. The method of claim 72, wherein said exogenous nucleic acid is, or encodes a microRNA or siRNA.

77. The method of claim 72, wherein said plant is selected from members of the angiosperm families Aceraceae, Anacardiaceae, Apiaceae, Asteraceae, Betulaceae, Brassicaceae, Buxaceae, Chenopodiaceae/Amaranthaceae, Compositae, Cucurbitaceae, Fabaceae, Fagaceae, Gramineae, Juglandaceae, Lamiaceae, Lauraceae, Leguminosae, Moraceae, Myrtaceae, Oleaceae, Platanaceae, Poaceae, Polygonaceae, Rosaceae, Rutaceae, Salicaceae, Solanaceae, Ulmaceae or Vitaceae or gymnosperm families Cuppressaceae, Pinaceae, Taxaceae or Taxodiaceae.

78. A plant with increased resistance to modulation of root architecture by the action of a root knot nematode, relative to an untreated or wild-type plant, obtained by the method of claim 72, or a part thereof.

79. An isolated plant promoter comprising a nucleotide sequence as shown in SEQ ID NO: 337.

80. A recombinant vector comprising the promoter of claim 79.

81. A transgenic host cell comprising the promoter of claim 79.

82. The transgenic cell of claim 81 which is a plant cell.

83. A transgenic plant comprising the promoter of claim 79.

84. A method according to any one of claim 1, 16 or 21, wherein said RAR comprises an RAR domain comprising an amino acid sequence (X₁)ₙX₂X₃X₄X₅X₆PGX₉SPGX₁₃GX₁₅ (SEQ ID NO: 459), wherein:
n may be 0 or 1
X₁ is selected from D, G, P, A, S, E and V;
X₂ is selected from F, V, R, T, S, A, K and Y;
X₃ is selected from R, K, E, H, Q, S, P, D, V, G, and A;
X₄ is selected from P and G;
X₅ is selected from T, S and G;
X₆ is selected from N, A, T, G, P, D, K and S;
X₉ is selected from N, H, Y and S;
X₁₃ is selected from I, A and V; and
X₁₅ is selected from N and H;
wherein
the amino acid at position 6, if threonine or serine, may be phosphorylated;
the P at opposition 11, a P at position 4, or both such prolines may be hydroxylated; and
tyrosine residues may be sulphonated.


SEQUENCE LISTING (496 sequences)

SEQ ID NO: 1 - DNA, 619 bp, Arabidopsis thaliana
acaccttatgttctccaatccaacacatatattttctttcacaaaaaaagacatattttaccttgttttgttttacatattcttatattttatcgatttgtctttgtccccggctcatgggaatgtcgaataggtcagtttctacatccatttttttccttgcattggtggttttgcatggaattcaggacacagaagagagacatttgaaaactacttcgttagagattgagggaatttataaaaaaactgaggccgagcatcctagcattgtggtcacatatacacggcgtggtgtccttcagaaggaggtcattgcccaccccacagactttaggccaacaaatcccggaaacagcccaggcgttggacactctaacgggcgacattgattcgatcatatcatacgtcataatcttatatcatatagaaaattacatgtattttcattcagacttgtcttctaatgctaaaggggtgtttggacatcactttatcatttcaatgttttgacagtactatgatcattatgtctttgtcgtgggtttgttacactgtcacgtattgtaagattatataatgaatgaattgcttttaaaaattaatatatacattaatg

SEQ ID NO: 2 - protein, 91 aa, Arabidopsis thaliana
MGMSNRSVSTSIFFLALVVLHGIQDTEERHLKTTSLEIEGIYKKTEAEHPSIVVTYTRRGVLQKEVIAHPTDFRPTNPGNSPGVGHSNGRH

SEQ ID NO: 3 - DNA, 381 bp, Arabidopsis thaliana
atgaagctattcattatcaccgtggtgaccattttgaccatctcaagggtatttgacaaaacaccagccaccactgaagcaagaaagtcgaaaaagatggtcggtcatgagcatttcaatgaatatttggatcctacttttgcagggcatacatttggagtagttaaagaagattttctcgaagtaaaaaagctaaagaaaattggtgatgaaaataatctaaaaaacagatttataaatgagtttgcgcctactaatccagaagatagtctcggtattgggcatccaagagttctaaacaacaaatttacaaatgattttgcgcctactaatccaggagatagtcccggtatcaggcatccaggagttgtgaatgtttaa

SEQ ID NO: 4 - protein, 126 aa, Arabidopsis thaliana
MKLFIITVVTILTISRVFDKTPATTEARKSKKMVGHEHFNEYLDPTFAGHTFGVVKEDFLEVKKLKKIGDENNLKNRFINEFAPTNPEDSLGIGHPRVLNNKFTNDFAPTNPGDSPGIRHPGVVNV

SEQ ID NO: 5 - DNA, 415 bp, Arabidopsis thaliana
aatacattttcgccttcgactaaatattataatttagcttctttttttttctaattctcgtctcggtttttctagtaatggcgacgattaatgtttacgtttttgcatttatctttcttttgactattagtgttggttcaattgaaggccgaaaactcaccaaattcaccgtaacgacgtctgaggaaatcagagctggtggctctgtattgtcgtcgtcacctccgactgagccacttgagtcgccgccgagccacggggttgataccttcagacctacggaacctggtcatagccccggtattggacattccgtacataattaacggagaggaacaatagcatcgtctatgtgattacatgttgaaaatatgattggcctggtgactttttttttctgaatatgtatttacgt

SEQ ID NO: 6 - protein, 82 aa, Arabidopsis thaliana
MATINVYVFAFIFLLTISVGSIEGRKLTKFTVTTSEEIRAGGSVLSSSPPTEPLESPPSHGVDTFRPTEPGHSPGIGHSVHN

SEQ ID NO: 7 - DNA, 261 bp, Arabidopsis thaliana
atggtgtctcgcggttgttcaatcacagttttgtttcgctttcttatagttcttttggtgatacaagtacactttgagaatacaaaagcagctcgacatgcaccagttgtttcgtggtcaccacctgagccgcctaaggatgattttgtgtggtaccacaagatcaaccgcttcaaaaacatagaacaagatgcattccgaccaacccaccaaggtcctagtcaaggtattggacacaaaaaccctccaggtgctccttaa

SEQ ID NO: 8 - protein, 86 aa, Arabidopsis thaliana
MVSRGCSITVLFRFLIVLLVIQVHFENTKAARHAPVVSWSPPEPPKDDFVWYHKINRFKNIEQDAFRPTHQGPSQGIGHKNPPGAP

SEQ ID NO: 9 - DNA, 690 bp, Arabidopsis thaliana
atggtattttaccaaacaccaatcaccactgaagcaagaagcttgaggaaaacaaacgaccaagatcattttaaagctggatttacagatgatttcgtgcccacttctccaggaaacagtcctggtgtgggacacaaaaaaggtaatgtgaatgttgaagggtttcaagatgacttcaagcccacggaaggaagaaagttgctgaaaactaacgttcaagatcatttcaaaaccggatctacagatgattttgcacctacttcccctggacacagtcccggggtgggacacaagaaaggaaatgtcaatgttgaaagttccgaagatgacttcaaacacaaggaaggaagaaagcttcaacaaacaaacggtcaaaatcatttcaaaaccggatctacggacgattttgcacctacttctccgggaaacagtcctgggataggtcacaagaaagggcatgcaaatgttaaagggtttaaagatgacttcgcacccacggaagaaatacgattgcagaaaatgaacggtcaagatcatttcaaaaccggatctaccgatgatttcgcacctacaactccaggaaacagtcccggtatgggccataagaaaggagatgacttcaaacccacgacaccaggacatagccccggggttggtcatgctgtcaagaacgatgaacctaaagcttaa

SEQ ID NO: 10 - protein, 243 aa, Arabidopsis thaliana
MKLLSITLTSIVISMVFYQTPITTEARSLRKTNDQDHFKAGFTDDFVPTSPGNSPGVGHKKGNVNVEGFQDDFKPTEGRKLLKTNVQDHFKTGSTDDFAPTSPGHSPGVGHKKGNVNVESSEDDFKHKEGRKLQQTNGQNHFKTGSTDDFAPTSPGNSPGIGHKKGHANVKGFKDDFAPTEEIRLQKMNGQDHFKTGSTDDFAPTTPGNSPGMGHKKGDDFKPTTPGHSPGVGHAVKNDEPKA

SEQ ID NO: 11 - DNA, 501 bp, Arabidopsis thaliana
acttcacatcaccactctcaaatattctcaagagtccttaacaacgatatttagttttttcccttttttctgttttctttttatctcccatttttcttccaatataatggaatcgtttatgggtcaaaagaaaacattgtacgcgtgttattttttaatgttggtgttttttttagggttcaattgtgtccatggacgaaccctaaaagttgatgataagattaatggtggtcattatgatagcaagacgatgatggcattggcaaagcacaatgatatgatggttgatgacaaggcaatgcagttctcgccgccaccaccaccaccaccgccgtcacaatcgggaggtaaagatgctgaagatttcaggcctacaacgcctggccacagccctggcattggccatagtttatcccataattgatcattttcatgcaatttcacatatgtatatatgtgttgtgaacttatgattaaatattgttcgttttaatttttctt

SEQ ID NO: 12 - protein, 105 aa, Arabidopsis thaliana
MESFMGQKKTLYACYFLMLVFFLGFNCVHGRTLKVDDKINGGHYDSKTMMALAKHNDMMVDDKAMQFSPPPPPPPPSQSGGKDAEDFRPTTPGHSPGIGHSLSHN

SEQ ID NO: 13 - DNA, 306 bp, Arabidopsis thaliana
atgaaactctcagtttatatcattcttagtattctcttcatttcgacggtattttatgaaattcagtttacggaggcgagacagttgcgaaaaaccgacgatcaagatcatgatgatcatcatttcacagtcgggtacactgatgattttgggcctacttctcctggtaacagcccgggcattggtcataagatgaaggagaatgaagaaaatgctggagggtataaagatgacttcgaacctacgacgccaggacatagtcccggcgttggacatgctgtcaagaacaatgagcctaatgcttaa

SEQ ID NO: 14 - protein, 101 aa, Arabidopsis thaliana
MKLSVYIILSILFISTVFYEIQFTEARQLRKTDDQDHDDHHFTVGYTDDFGPTSPGNSPGIGHKMKENEENAGGYKDDFEPTTPGHSPGVGHAVKNNEPNA

SEQ ID NO: 15 - DNA, 589 bp, Medicago truncatula
atgaaatatttcgctctttttcttgccctaattgcatgcaactattcccttcaatctcatgcaaggctcattaaaccatcgaaccatcacaatgttccaatttcaacatcagagaagaaagttgagtcaacaataaaatcaaacaatgaagtagctagttattttggagattcaagtgaagctcacacaaatgcattccaaccaacaacaccaggaaatagtcctggtgttggtcatagatattttaccgatgaagatatcgacgtgaattcgaaaaagacggtagctcagagcaaagatgataataaatatgtgactgaggatactacaaatgagttccaaaaaacaaaccctggtcacagtcctggtgttggtcattcttaccaaaacaaaattggaaattgatgtaaatatgcaattaataatttattttattagttaggtgtatgctgtataattaatcaatcaattaattattaagtgttctccatagtttcattctgcattacagattgtgaaattcttgcatatcccaaaccatgagcttgggtttaattaattattggctattgattgtatcattcaattc

SEQ ID NO: 16 - protein, 142 aa, Medicago truncatula
MAYKFQYTMKYFALFLALIACNYSLQSHARLIKPSNHHNVPISTSEKKVESTIKSNNEVASYFGDSSEAHTNAFQPTTPGNSPGVGHRYFTDEDIDVNSKKTVAQSKDDNKYVTEDTTNEFQKTNPGHSPGVGHSYQNKIGN

SEQ ID NO: 17 - DNA, 249 bp, Medicago truncatula
atggcacatttagctcgcatttgcttgttctatgtactattgtttttgtctcatgaactactactcactacaactgagggtaggagtttgagacaaagcattcagccaccaaacatagcctctaccaaaatgatgagcacaagccaattgtaccaccgtagcaatagaagtttggagggagatgttgaagcttttaggcccacaactcctggacacagtcctggcattggtcattccattaataattaa

SEQ ID NO: 18 - protein, 82 aa, Medicago truncatula
MAHLARICLFYVLLFLSHELLLTTTEGRSLRQSIQPPNIASTKMMSTSQLYHRSNRSLEGDVEAFRPTTPGHSPGIGHSINN

SEQ ID NO: 19 - DNA, 640 bp, Medicago truncatula
ttttaaagtatattggtacatgtgtaacacgacccaaaataatttttctaaaaaggaaatgaaaaaaagagaaaatgaacatccaaaacaatcaacgttgagctccaccattttgcagcagtttatttaaattagtaacctggctcggcagatcaatcattaataggaggtggagcgctcatgcgacattctacttcttgtgtccagcacctggactatttcctggtgttgtgggacgaaaatcattcgtctcaagattcttgtaacgtttcaatttgtgagaatggatatcggattgatcgggagcaaaagtgtgaatatctggaagatgactagcagctttgtcttgattaacactaacggataatgcaacatgcttgtgatactcacacaccgaaattatgtctgtcggggaaatcttggctttaactaataacgccttagtgccgtgacatgacatcagatcccttagagctcgattatatgacattaatatagctttttccgagactacagatggactcaaggatatgaaagccaatatggccaacaacccagccaaacaatattttggaccataactcatgttttctagcgcaatgcttattaagtgataatgcaagcacttttatattttttt

SEQ ID NO: 20 - protein, 131 aa, Medicago truncatula
MSYGPKYCLAGLLAILAFISLSPSVVSEKAILMSYNRALRDLMSCHGTKALLVKAKISPTDIISVCEYHKHVALSVSVNQDKAASHLPDIHTFAPDQSDIHSHKLKRYKNLETNDFRPTTPGNSPGAGHKK

SEQ ID NO: 21 - DNA, 243 bp, Medicago truncatula
atgggtgagaaaaccatgttattaacatttttacttcttattattatgcaacaaaacattggttcaattgaagcatcaaggttgctaaatattaatccaccaccaactattcctaaaagtccacaagctccttcacatgattattggtattcgataaacgatgataagggtggtgacgatgcttttcgccctacaagtccaggacatagccctggggtaggacatcaaacaccacctccatga

SEQ ID NO: 22 - protein, 80 aa, Medicago truncatula
MGEKTMLLTFLLLIIMQQNIGSIEASRLLNINPPPTIPKSPQAPSHDYWYSINDDKGGDDAFRPTSPGHSPGVGHQTPPP

SEQ ID NO: 23 - DNA, 249 bp, Medicago truncatula
atggaaaatactaaaaggcttcaaattatttgtgttcttattttgtttttggttttgcaacaagaagttgtgattgttcaagggaggcatttgaggtctaaattgtgtagagattgcacaaagcctcataaaagatccattgctcatcatggagggaagtcttcaagacgtgtagggtatgaagttgatgattttaggcctacatctccagggcatagtccaggtgttggtcattccatccataattaa

SEQ ID NO: 24 - protein, 85 aa, Medicago truncatula
MAHFTRSCLIFVLLLISCELLSIEGRSLRKSIGSPKAASVETMTRSVVLSPRQLQNNGRNLEGSVEAFRPTTPGHSPGVGHSLKN

SEQ ID NO: 25 - DNA, 249 bp, Medicago truncatula; identical to SEQ ID NO: 23

SEQ ID NO: 26 - protein, 82 aa, Medicago truncatula
MENTKRLQIICVLILFLVLQQEVVIVQGRHLRSKLCRDCTKPHKRSIAHHGGKSSRRVGYEVDDFRPTSPGHSPGVGHSIHN

SEQ ID NO: 27 - DNA, 375 bp, Medicago truncatula
atgggtttgtttcaagtcacaacaaaatatttgatcgttattctagcactaagtattgtatacaattcctttcaaataactcaagccaggccaattaaaccattgaatcaacaatcttcattaaacacacaagactcgggtgcaatccacactaactcttttcggccgacaacaccaggaagtagtcctggtgttggccaccgaaattttgttgtaggagataagaacacgagaacaatggtggttgttcagagcccggatgttgaggtttttgtgacgaataagagatccgatgatggtttcaaacctacaaatcctagtcatagtcctggagttggccatggttaccataccaaaattagacatttaaattag

SEQ ID NO: 28 - protein, 124 aa, Medicago truncatula
MGLFQVTTKYLIVILALSIVYNSFQITQARPIKPLNQQSSLNTQDSGAIHTNSFRPTTPGSSPGVGHRNFVVGDKNTRTMVVVQSPDVEVFVTNKRSDDGFKPTNPSHSPGVGHGYHTKIRHLN

SEQ ID NO: 29 - DNA, 309 bp, Medicago truncatula
atggcacaaaacaagaccatagttttctctgttatttccctagcattgatcattttctgcatgcagtcgatcgaggggcgccttgtaaaatacatcgatgaaagtaacctcctgaagaatgttaaacatgatggaatttcagatgcaaatgaagctactcttgttaacgtgactccaacaatattgccaccaagtgctgtggtaggttcaaatggggttgcagcacctcctccaagtcatgatgtgggtgcttttagacccacaacccctgggaacagtcctggtgtaggtcactctattcactactag

SEQ ID NO: 30 - protein, 102 aa, Medicago truncatula
MAQNKTIVFSVISLALIIFCMQSIEGRLVKYIDESNLLKNVKHDGISDANEATLVNVTPTILPPSAVVGSNGVAAPPPSHDVGAFRPTTPGNSPGVGHSIHY

SEQ ID NO: 31 - DNA, 456 bp, Medicago truncatula
atgggtgaatttcaggccatgcaaaaatattttgccatttttcttgtattagttgcctaccatattttccttccaactcaagctaggaagataaaaccattgattgaagataatcccaaacctaccttcacatcccttaaaactgctgtaaatattccttctccaacatttgagaagaaagttaaccttcccatgatgccaaatcatggtgtcgcaagtataggagattcaagcggagatacaaatgctttccgacccacaacaccaggaagcagtcctggtgttggtcatcggaagtttgtaggagaggttaaagatagtacagttgttcggagtccgaatgttaaagtttttgtgacttctgagagatcaaaagatgcttttaaacctacttacccaaatcatagcccaggtgttggacatgttaaccaaagcacaaaaggacaactaaattag

SEQ ID NO: 32 - protein, 151 aa, Medicago truncatula
MGEFQAMQKYFAIFLVLVAYHIFLPTQARKIKPLIEDNPKPTFTSLKTAVNIPSPTFEKKVNLPMMPNHGVASIGDSSGDTNAFRPTTPGSSPGVGHRKFVGEVKDSTVVRSPNVKVFVTSERSKDAFKPTYPNHSPGVGHVNQSTKGQLN

SEQ ID NO: 33 - DNA, 573 bp, Medicago truncatula
atggctgagaaaatcatgtttgtaacctatttacttatccttattattatgcaacaataccttggatcaatggaagcatcaaggtttataaatgataataataaggatggtgatgatgctttccgtccaactccttcaggtcatagtcttggggtgggacatatattaccaccaccatcaagtattatccctaaagtcttattgaaaagtcaacaacctccttcatctgattatttgtataccataaaggatgataataaggacggtgatgatcctccatcacatgattattggtattccataaatgatgataataaagatggtgatgatgctttccgtccaaatcctccaggtcatagtcctggagggggacatacgttaccaccatcaccaccaagtgttatccctacagtcttattggaaaatccacaacctatttcatctgattatttctataacataaaggatgataataaggatggtgatgatgcttttcgcccaactcctcctggtcatagccctggagggggacatacattaccaccatcaccaccaattgtttttatgaactaa

SEQ ID NO: 34 - protein, 223 aa, Medicago truncatula
MAEKIMFVTYLLILIIMQQYLGSMEASRFINDNNKDGDDAFRPTPSGHSLGVGHILPPPSSIIPKVLLKSQQPPSSDYLYTIKDDNKDGDDAFRPTPPGHSPGGGHTLPPSPPSIVPIISLKSLQPPSHDYWYSINDDNKDGDDAFRPNPPGHSPGGGHTLPPSPPSVIPTVLLENPQPISSDYFYNIKDDNKDGDDAFRPTPPGHSPGGGHTLPPSPPIVFM

SEQ ID NO: 35 - DNA, 258 bp, Medicago truncatula
atggcaaagaaaaccattatgttaagctttcttgtttttctcattcttgtgcaaaattttggtttgatggaagtgctagggaagaatgttgaagcaccaccaacaattccaagagttttgttgaggagtccacaagctccttccattggcttttataccaaaaatgatgacaaggatagtcaaggtgatgcttttcgtccaactagtcctggtcatagtcctggtgtgggccatgattcgccaccaaattttccttaa

SEQ ID NO: 36 - protein, 85 aa, Medicago truncatula
MAKKTIMLSFLVFLILVQNFGLMEVLGKNVEAPPTIPRVLLRSPQAPSIGFYTKNDDKDSQGDAFRPTSPGHSPGVGHDSPPNFP

SEQ ID NO: 37 - protein, 105 aa, Arabidopsis lyrata
MESSMGQKKTLYACIFLMMVFFLGFNCGHGRTLKVDDKIDGGHDDSKTMMALAKHNVMMVDDKTVQFSPPPPPPSPSQSGGKEAEDFRPTTPGHSPGIGHSLSHN

SEQ ID NO: 38 - protein, 231 aa, Arabidopsis lyrata
MKLLSITVMTIVISMVFDQTPITTEARRLRNTNDQDHFKAGSTDDFAPTSPGNSPGVGHRKGKVNVEGFQDDFKPTEGRKLLKTNGQDHFKTGSTDDFAPTSPGHSPGVGHRKDTANVERFQQTNGQNHFKTGSTDEFAPTSPGNSPGIGHKKGNANVKGFKDDFAPTEEIRLKKMNGKDHFKSGSTDDFAPTTPGNSPGMGHKKGDDFKPTTPGHSPGVGHAVNNNEPKA

SEQ ID NO: 39 - protein, 90 aa, Arabidopsis lyrata
MGMSNRSVSTSLFFLALVVLHGIQDTEERHLKTTSLEVEGIYKKTEAENPSIVVTYTRRSVLQKAVIAHPTDFRPTNPGNSPGVGHSHGR

SEQ ID NO: 40 - protein, 92 aa, Solanum lycopersicum
MAQPKIMYTCAFFLALIFFSYGILLSEGRVLFKKEKNNNTIFSHHEENSHTKVVKNNYFNNIDHNNMHDNINISEEGGPGHSPGVGHGGGPP

SEQ ID NO: 41 - protein, 81 aa, Solanum lycopersicum
MASSYQKSIYMVIFYVFLFLFLHQCELIVASRVVVMKFHQPMMPPSTNILSFNRYKKSEIVKDYSGPGHSPGMGHNDPPGA

SEQ ID NO: 42 - protein, 74 aa, Solanum lycopersicum
MASSYKKSIYMVLFYVFVFLLLQQCELIVASRVVVMKFHQPKPPSTNIFSFNRYKKSEVVKDYSGPGHSPGMGH

SEQ ID NO: 43 - protein, 82 aa, Lycopersicum esculentum
MASSYQKSIYMVIFYVFLFLFLHQCELIVASRVVVMKFHQPMMPPSTNILSFNRYKKSEIVKDYSGPGHSPGMGHNDPPGAP

SEQ ID NO: 44 - protein, 120 aa, Lycopersicum esculentum
MVIVTNTKIIQFFAFILVLILFSHEILCVEAIRHLKSEKMEVVSVEISVSSTQIVVTSETFNKIGKIQKSLTWLPSKDDIHKSINDPTEATKSVKVVEKMDDFGPTGPGHSPGIGHSIHS

SEQ ID NO: 45 - protein, 93 aa, Gossypium hirsutum
LFLFQPLKWPKPTSLYPLPFSPSFCLLTGSQFSKEARVLKADHKTHHHSSLNVNVKGDVLPDGSATVNNVQKAAYRTDAFRSTTPGHSPGAGH

SEQ ID NO: 46 - protein, 103 aa, Lactuca sativa
MVHFQIYPCVFFLLIISFHGLIPLFEGRKLKDVTAFRPTTPGNSPGAGHSFTENRPYFRSKEVESKDSGIHHPNSESATGFRPTKPGNSPGAGHSIHNQTAMP

SEQ ID NO: 47 - protein, 210 aa, Euphorbia esula
MAEIQKFVIFLLAIVFYLQSQSTSARPVKFVNKKGLALKKNSDSFKLHQTMKKEQMPPPVDKTGFFGDFSDKSTDDFRPTSPGYSPGVGHPKAVFANSQSDRIDHSTARKEEESTTDDFRPTEPGYSPGVGHPMEASTSSDKDDYRPTEPGHSPGAGHPKEESTDDFRPTAPGFSPGVGHRKEVVTVPEAENDFSGTKDDYRPTQPGHSP

SEQ ID NO: 48 - protein, 232 aa, Euphorbia esula
  misc_feature (212)..(215): Xaa can be any naturally occurring amino acid
ISKSPNAIYPPATSISFDDEEEEPQEAHVYAFRPTAPGHSPGVGHKEELEDAHLYAFRPTAPGHSPGVGHKEEPEDSMNSHVIISKSPNAIYPPTTSISFDEEEEPQEAHLYAFRPTAPGHSPGVGHKEELEDAHLYAFRPTAPGHSPGVGYKEEPEDSMNSHVRISKYPNAIYPPTTSISFDEEEEEPQEAHLYAFRPTAPGHSPGVGHKXXXXDAXXYAAIAKGLATPSS

SEQ ID NO: 49 - protein, 158 aa, Glycine max
MAKFQVLHEYFFIFLALVVCDGSLLTHGRKINIKPLNQLHSSLNTKTVANHPNPTSLPSLKTKVESPQHHEESSKLEDSGADNTNAFRPTTPGGSPGVGHKMITSSSEDNKVKTMVVVHSPDVEVFKTEGSKDDFKPTDPGHSPGVGHAYKNKIGDEN

SEQ ID NO: 50 - protein, 87 aa, Glycine max
MAHFTRTCLLLVLLFLSCELLCIEGRGLKATTKSPKSVSVRAMSTTKGAVAKPSQLETIAKSLNGFVEAFRPTTPGHSPGVGHSVNN

SEQ ID NO: 51 - protein, 144 aa, Glycine max
MHKYFTIFVALFACHGSLFAHGRQIKPLNQHSSLNTNPILAPLSRTSIKVIEAPIVPKFKFSDVDSGDSGADHANAFRPTTPGNSPGVGHKKFEEDKVMKVMGALVHSPDVKTSVAEGSFENDFKPTDPGHSPGVGHPRQNKRN

SEQ ID NO: 52 - protein, 94 aa, Glycine max
MAQHKFLLCLILLALIIFCQGLHSIEGRYLKSDHEIIKHQYQMHSGISTTNVAALVADVSPPTPPSAAVPGRDNDNFRPTAPGHSPGVGHAAHN

SEQ ID NO: 53 - protein, 141 aa, Glycine max
MAAQVLHKYFFIFLALVVCHGSLVAHGRKINVKPLNQQHYSLNTKTVANNNPYPSLPSLKTKVESPQYEEANKLGDSGSTGVGHKIITSSEDNKMKTMVVVQSPDVEVFVTKGSKDDFKPTDPGHSPGVGHVYQNKIGQAN

SEQ ID NO: 54 - protein, 86 aa, Glycine max
MENSSLRNIAFVLFLFLILHHQVLFVQGRNLKCPLCKECSKSQKNTMSVASYEVHQEGLRRVEYEVDDFRPTTPGHSPGVGHSINN

SEQ ID NO: 55 - protein, 109 aa, Glycine max
MANSKLGFNFMVSAIFLSLMTFHGTFSVQGRPLKMEIKEQVTTHENIIDEIAKAAEYTATWHRHTLEFEDTKNPQYDGVTNDFQPTDPGHSPGAGHSSPHANIVSISKP

SEQ ID NO: 56 - protein, 75 aa, Glycine max
  misc_feature (75)..(75): Xaa can be any naturally occurring amino acid
IKLIDAPIVPKFKFADVDSGDSGADHANAFRPTTPGNSPGVGHKKFEGEDKDAGSFENDFRPTDPGHSPGVGHPX

SEQ ID NO: 57 - protein, 87 aa, Glycine max
MAQNKFLLSLVLLALIIFCQGFHSIEGRYLKSGETIKHQMHSGISTTNVADVSPPTPPSAAVPGRDVDNFRPTAPGHSPGVGHTVHN

SEQ ID NO: 58 - protein, 108 aa, Glycine max
MANLKLVFTMSSILLVLVFFNGILPAMGRPLKKEHITTTYENSVKEMGTVEDNNILLWRRSIIENNAANDGGVDKWIDDFRPMDPGHSPGAGHSSPTPKDATNGAPRP

SEQ ID NO: 59 - protein, 81 aa, Glycine max
MAQKIIWLTFLVFLILQHNFGTMEASRKLIHTHPPPAIPRSPQAPALWYTPNDEDGGHDAFRPTCRGHSPGAGHDNPPTKP

SEQ ID NO: 60 - protein, 81 aa, Glycine max
MARLTHFVLLFVLLFLSHELLGSEGRNLRQITIQSPDATKAMSIATKSANAIPSYRSIRSLSGDVEAFRPTTPGHSPGVGH

SEQ ID NO: 61 - protein, 87 aa, Glycine max; identical to SEQ ID NO: 50

SEQ ID NO: 62 - protein, 163 aa, Glycine max
MAIFQYATRKCLVIFLLLVAFNGSLLTHGRQIKPLNQQHSSLNNDTVVKHSVNNVPTHPSSGKKKVVDSSSVVPKYGVESFGDSMSSDTNAFRPTTPGNSPGVGHRKFAPEDKDVEAMVASVQSPDHVKVYVTEGTQNQDGFKPTNPGHSPGVGHAQQNKIGQ

SEQ ID NO: 63 - protein, 76 aa, Glycine max
ESPQHHEESSKLEDSGADNTNAFRPTTPGGSPGVGHKMITSSSEDNKGSKDDFKPTDPGHSPGVGHAYKNKIGDGN

SEQ ID NO: 64 - protein, 108 aa, Glycine max
MTNLKLVFTISSILLALVFINGISSVMGRPLKKEHIITTTYENSVKEMGTVEDNNILLWRRSIIENAANDGGVDKWIDDFRPTDPGHSPGAGHSSPTPKDASNGAPRP

SEQ ID NO: 65 - protein, 95 aa, Lotus japonicus
MTNSKKLVFTISSILLLTLMFSNFIFSAHGRPLKTENKEHVTTYENNSVKEMATGENDHKVGKLINDFKPTDPGHSPGVGHSSPIPMDANEPPRS

SEQ ID NO: 66 - protein, 89 aa, Lotus japonica
MAQNKPIFSLILLALIIFCHGFQSIEGRYFKIGEGTQHLMKHGDFSTTNGVVSGASEAPSLTPSRDVSGFKQPTTGPGHSPGVGHSIHN

SEQ ID NO: 67 - protein, 99 aa, Lotus japonica
MAKTNLKFVCVVFLLLILHHQHVCVQGRHLRSCLCRGCPKTCVKIKSGVAHGVGDRGNRATTHDYDTHQGRKRLVEYEVEAFRPTSPGHSPGVGHSINN

SEQ ID NO: 68 - protein, 86 aa, Lotus japonica
MEEKTVMLTLLVILILQHNYGSMALSGNNIHPPPAIPRALLRSPQPPSPGWYTINDDKVGEGDAFRPTTPGHSPGVGHDSPPNFHA

SEQ ID NO: 69 - protein, 91 aa, Lotus japonica
MAHFARTCLLFVLLFVSCELLCIEGRTLSKNVLDHSLKSSSVKAMSIATVKTENGVVASPSQLRRSMEGYVEAFRPTTPGHSPGVGHSVHN

SEQ ID NO: 70 - protein, 125 aa, Lotus japonica
MGEFQARTIYFLVFLALFACNCSLLCHGRPLKPVNSPIMPNQDVATSGDAAGASYTNAFEPTTPGNSPGVGHRSFAGEDNKMVAAQSPDVGVSVTQGSESDFKPTDPGHSPGVGHAYQEKIGHLN

SEQ ID NO: 71 - protein, 210 aa, Populus trichocarpa
MPHPAVPSFGNSAAVYKDDFRPTTPGVSPGVGHPKTIGTNSNNEHSLTDFKDDFQPTTPGHSPGAGHALANDDDNEEVSPKAPGPSIERSGTAFKPTTPGHSPGAGHALANDDDNEEVSPKAPGSSIERSGTAFKPTTPGHSPGIGHLFSENDTDKNEITASKASSIEHSVTGVTDDFRPTVPGHSPGIGHAFRPPTPGHSPGVGHSIHN

SEQ ID NO: 72 - protein, 370 aa, Populus trichocarpa
MAETCKCAFLILAFVTCFQILFIEGRSIKQTNKQEHVTNEIEPLKEMANQSTNTNLHHNTANNQKVSLPSPPVHIPTVHHSKAGRKEMTPPMVPSFSGSPGVRHPKTPGANSVTTVKDDFKPITSGQSPGVGHNNDNSVTAFKDDFQPTTPGNSPGVGHILVDEDDSEDDDPKAPGTSSSNERSGAAFKPTTPGHSPGVGHMSSVDQSDKTDLKASKTELSVTTPAFKPTTPGHSPGVGHMSSVDQSDKIDSKASEIEHFNTEHSVTTPGHSPAVGHILSDEDEDDNEDVDPKAPGTGSSIKRSGAAFKPTTPGHSPGIGHMSSVDQSDKTDRKATNIEHSVARVPDGFRPAVPIQGPGVGHVFQAQTKN

SEQ ID NO: 73 - protein, 158 aa, Populus trichocarpa
MIQRHPVLAPVMKRSGAAFKPTTPGHSPGIGHMSSVDQTFKPTTPGHSPGIGHMSSVDQSDKTDSKASEIKHSVTTPGHSSRVGHILSDEDADDTFKPTTPGHSPGIGHMSSVDQSDKTDRKATNIEHSVARVPDGFRPAVPIQGPGVGHVFQAQTKN

SEQ ID NO: 74 - protein, 90 aa, Populus trichocarpa
MADKTRSFMLTFFTVVLLLLHQHFDLTAASRPLDIHSPAIPRSGSEPPPTDVHDRWYRINRYKNLESDAFRPTTPGHSPGVGHENPPAAP

SEQ ID NO: 75 - protein, 88 aa, Populus trichocarpa
MAKGKLIFTSTLIIVLVLCYGITSSVGRLLKTGENTSSFSLHRDLLVSEARSEPVTPGPDHADADSDDFKPTTPGHSPGAGHSTPGHN

SEQ ID NO: 76 - protein, 70 aa, Populus trichocarpa
MAQSNLLSAFVFLVLIFSHELQFIEGRYLNLKTPNKFLQKEIRRLVESNSKLHVNDNLDKPVNATKVAPP

SEQ ID NO: 77 - protein, 244 aa, Vitis vinifera
MAIIQVIHACSLLLAVITYHDILYTEGRPINSVTKQEFSSTDFEPGNETGSQGTEHKEDHWYTPPPPEPNPSVKNSVVGKDILPPITPNYSIGFGDSTAVYKDGFRPTTPGSSPGIGHQFVPTKEDIQPKALGNSPSVRHSVTAYKDDYRPTMPGHSPGEPNPGVKNSVAGKKELPPPMLPNYSVGFGDSTAVSKDDFRPTTPGSSPGVGHHSDDYRPTKPGHSPGVGHSLQKTNAEPNAEPNA

SEQ ID NO: 78 - protein, 140 aa, Vitis vinifera
MAKIRFIHAYSLLLAVITYHHILCTEARPIKSPSSIDYEPGKETGSQGTEHKDVWSGPPPPEPNPIVKNSVAGKEEILPPMIPNYSVGFGDSAAVHTDGFRPTTPGSSPGIGHSSAPTKEDIEPKAPGNSPKTFRQGKQG

SEQ ID NO: 79 - protein, 140 aa, Vitis vinifera
MAKVHIIHACSLLLAVITNHDILYTEGRPMKSLSKHEFSSIDSGPGTETGSEGIEHKDDHRSAPPPPEPNPGVKNSVAGKKELPPPMMPNYTTGLADSTAVYEDDFRPTPPGSSPGIGHHFDFRPTTPGHSPGVGHSLQN

SEQ ID NO: 80 - protein, 110 aa, Vitis vinifera
MAKVKLIMSIYVFILALVLIYGGLMSEGRKLDIEKNSKCEMCVSIDEKISVLGNLHRSSKANARPHAPARQSPGADRLFTDDGVDVQSTTPGHSPGVGHSVGPASNDPNP

SEQ ID NO: 81 - protein, 166 aa, Vitis vinifera
MDRQDAILDIQWLKKILVRHRVITNHDILYTEGRPMKSLSKHEFSSIDSGPGTETGSEGIEHKDDHRSAPPPPEPNPGVKNSVAGKKELPPPMMPNYTTGLADSTAVYEDDFRPTPPGSSPGIGHHFVPTKGDIQPKAQGNSPGVGQSVTAYKDDYPPTKPARSQP

SEQ ID NO: 82 - protein, 173 aa, Vitis vinifera
MAKIQVIHACSLVLAVITYHDILYTEGRPIKSLDKHEFSSIDSEPGTETGSQGIEHKDDHWAAPPQEPNPGVKNSVAGKKELPPPMLPNYSVGFGDSTAVSKDDFRPTTPGSSPGVGHHSVPTKDDTQPKALRNSPSVRQSVTAYKDDYRPTKPGHSPGVGHSLQKTNAEPNA

SEQ ID NO: 83 - protein, 89 aa, Vitis vinifera
MANTRFLGACAVLLVLLLCHEFSCVKGRHLRSAMCKKCSRHRQTSLRATEAGEAPSGLPQMSTSKMEHIEDFRPTSPGHSPGVGHSIHN

SEQ ID NO: 84 - protein, 267 aa, Ricinus communis
MRKQLEAFQKELAKRGVSNTINLHQSKLAGQDHQEQTHTGFSDFAAASVDAFRPTPPGNSPGVGHPKAVVTSSSTTDQHSLTGLRHDYSNLHKSSHNIPGNVQQSMSGKEETSPTSLDVFAAASTDDFRPTSPGYSPGVGHPKAVVTSSSTADQHSFTGVKDYYNNVHKSNHIGVADNVKKPVSGKGEMLPTVTTTSFDASAASTKDDFRPTAPGFSPGVGHPKKVVTSSSTKHSITGFKDDYRPTQPGHSPGVGHSYQKNNAGQDP

SEQ ID NO: 85 - protein, 101 aa, Ricinus communis
MAIAASAAATNLMGTCTCLLVLILCHEAIYVVEGRHLKPKLCKKCSRRSESSLDVSKDGHHNTTTHLLNGDQEKISKMDFVDDFRPTAPGHSPGVGHSIQN

SEQ ID NO: 86 - protein, 95 aa, Ricinus communis
MANVCYTCLFFLVMLLSYDLVCIEARQLKLRENMKCVKCLSAPDSKESITRNPRGDNAMSSSQDGIEPKDGSNNFDAFRPTNPGHSPGVGHSIQH

SEQ ID NO: 87 - protein, 86 aa, Ricinus communis
MARVKLNFSIVLVIALVVSYGITSTEERQLRMQVRAAGMEKGTGNLYFGRSLLVDNDGDSDDFRPTNPGHSPGAGHSTGPSSKNAH

SEQ ID NO: 88 - protein, 123 aa, Ricinus communis
MAQTNLLFGCIFIMLIFFQELQSISGRHLNLETNHKFSKIQVSYINFERQHRQFIGHNVDIEHNDLNKDVFAANKMSPAAPVAAAGGIGEAESPPPPPASGHVDDFRPTAPGHSPGVGHSIQN

SEQ ID NO: 89 - protein, 89 aa, Ricinus communis
MANRAFLLTLFAISFLLLHQHLDSAVASRPLHMHPPAIISQGSLKRPLPPSTALLYSINRHKFTETEAFRPTAPGHSSGVGHGNPPAAP

SEQ ID NO: 90 - protein, 267 aa, Ricinus communis; identical to SEQ ID NO: 84

SEQ ID NO: 91 - protein, 61 aa, Ricinus communis
MKCVKCLSAPDSKESITRNPRGDNAMSSSQDGIEPKDGSNNFDAFRPTNPGHSPGGHSIQH

SEQ ID NO: 92 - protein, 70 aa, Ricinus communis; identical to SEQ ID NO: 76

SEQ ID NO: 93 - protein, 84 aa, Casuarina glauca
MAHRTLMLTLSLVILLLQQTIVSVTASRPVSIHPPDVLRGSLSIPKPPSTEWFTVNRYKKLEDAFRPTSPGHSPGVGHGTPPAA

SEQ ID NO: 94 - protein, 124 aa, Artificial Sequence
  Synthetic Construct
MEFRRMHTFAVFLLIACYLVLSVEGRFLKSLSKNNSKQVLPPPTPTKASDFGDSIEGYKEDFRPTTPGNSPGVGHSFADVVEDIVEQNPASISVQGNGKRSIAVHSPGVGHSFADVVEDIVEQN

SEQ ID NO: 95 - protein, 360 aa, Jatropha curcas
MAETLVSYKWTLFLLALISWLQILFSQARPIKSTDIHQSSNDNFLPKAPAGFTSPKGANPVTSSSADDFRPTTGGHSPGAGHPKKMVTSSDVEHSVTKPEADGRTVKLHQNKLTGTTTASTANDFRPTKPGYSPGVGHPKQIVTSSNIEHSITGFKATKPVLGSDTYNLHQNKLTGTTMASTTNDFRPTSPGYSPGVGHPKKIDASSNVEHSVTGFKANIAVGGTDNLHQNKLTGTTIASTTNDFRPTSPGYSPGVGHPKKVDASSNVEHSVTGFKANIAVGGTDNLHQNKLTGTATASTTNDFRPTAPGYSPGVGHPKAVLVPSSTNSNVDDYRPTQPGHSPGVGHKKSSDLVPNPETG

SEQ ID NO: 96 - protein, 107 aa, Theobroma cacao
MAKTNLIVLAGALLLVLLFSYGITFTEERVLKTDKDVKPAGNYVTNVMTSSHKTNLNRDILEDGTVDVPTSSSGNGTAFDADDFRPTTPGHSPGAGHSTGPASNDKN

SEQ ID NO: 97 - protein, 107 aa, Theobroma cacao
MAKTNLVVLAGALLLVLLFSYGITFTEERVLKTDKDVKPAGNSVTNVMTSSRKTNLNRDNLEDGTDDVPTASSGNDTAFDADDFRPTPPGHSPGAGHSTGPASSDKN

SEQ ID NO: 98 - protein, 107 aa, Theobroma cacao
MAKTNLIVLAGALLLVLLFSYGITFTEERVLKTDKDVKPAGNYVTNVMTSSHKTNLNRDILEDGTVDVPTSSSGNGTAFDANDFRPTTPGHSPGAGHSTGPASNDKN

SEQ ID NO: 99 - protein, 121 aa, Theobroma cacao
AEGVLGTAKDVNPGGKFVTNVAAGRHKTNLIRAFLEDGTVDVPTSSSGNGTAFGANDFRPPTPGHGPGAGHSTGPASNDKNWIPLPARTIIFPLPWVATFTQSLVGYISYDFVLALPKALK

SEQ ID NO: 100 - protein, 96 aa, Malus domestica
MANGKLSFLLLVLISSYGIISTEERFLKTDHTNGGSTSMISHDNYLNSRRNVFENELSDSVPPVPGYHSASDYRPTTPGHSPGAGHSVGPQVEPNQ

SEQ ID NO: 101 - protein, 153 aa, Carica Papaya
MEYQTIVRCGILLALLFASLMITEARKIRELITGNNGDFDDSFAAHDTAGFRPTTPGISPGVGHSFQNGNKDMSGSKAAHFKPPSSDYQKETSPPRAPKAPGNSPGGIGDSFADVNSQGWSNKDDFQVTVQATSPGHSGGVGHGDNDDEPNAR

SEQ ID NO: 102 - protein, 70 aa, Carica papaya
MANVACSCLFLVVMILCSHCLHGTQGRNLKNTPSSSKNMNFPKPSSVKSTEAIVEAFRPTTPGHSPGVGH

SEQ ID NO: 103 - protein, 114 aa, Carica papaya
MARTGLMGVCVLFLVLLVCQEIVFVNARHLRDRILCEKCSTTHHHRHHHHHHHHHHHLDKIRLSVAPANGAGPVHVNDGAGSEQQRWSTKDEYVDDFRPTTPGHSPGVGHSIGN

SEQ ID NO: 104 - protein, 151 aa, Fragaria vesca
MEAKCAVVFALIACLDIASVEGIRPFWSETKSTETILIDSIEANYKRELGEQSGQHNNLKGEFKSAVVKNQGHFAKLGAPAYNDEEDFRPTTPGNSPGAGHKSLQVSEPKTVVVAGRNYFTAGTKEDYRPTQPGHSPGVGHALQENVKPMP

SEQ ID NO: 105 - protein, 101 aa, Fragaria vesca
MANATYTCLFFLLVIFSHELISCTEGRNLKVTSKKLKCGKCLSPDIDAKSIAGDQGSGGSSSSNQIQSPPVVPLPASPGRVEAFRPTTPGHSPGVGHSVHN

SEQ ID NO: 106 - protein, 103 aa, Fragaria vesca
MAKRRQTRLIATTTTCTLLIVLICCHEITLVDGRHLKPGDCKKCSRRHRELNTLSAAKVGDHNRSARLVRAAETKTSKAEHVTDDFRPTTPGHSPGVGHSINN

SEQ ID NO: 107 - protein, 87 aa, Prunus persica
MVSFFMVALLLGQNSDLVAASRPLHLHTHPPAIHIGSLNKPIPPSIGRFTINRYKMTESSSGADAFRPTSPGHSPGVGHQDPPGALL

SEQ ID NO: 108 - protein, 91 aa, Cucumis sativus
MAHRSLHLNSFFPLVALLLLLLLLHSLFVTSSRPLHGIHPHNPHAITPPAPVSLETSFSINRYKYVETDAFRPTSPGHSPGVGHNEPPGKP

SEQ ID NO: 109 - protein, 155 aa, Manihot esculenta
LHCFCCRCFRPTDPGNSPGVGHHLSQEESDEETDPKPPRKDYGPKPGHSQPVGRDIIFSNPSNTKGSQPASSSHNPVNAVPLTPTAFDASAASSMEGFRPTTPGYSPGVGHPNAEISSSNVETSVTRFEDDHRPTQPGHSPGVGHAYLENNAEPN

SEQ ID NO: 110 - protein, 80 aa, Citrus sinensis
MLTLLVVLLLSKSFDLISASRPPHIHPPTIPRGSLLNKVKPPSFHAYTANRYKLTESEAFRPTSPGHSPGVGHKGPPGSD

SEQ ID NO: 111 - protein, 80 aa, Citrus clementina; identical to SEQ ID NO: 110

SEQ ID NO: 112 - protein, 81 aa, Catharanthus roseus
MANISCKCLFMIFLLILVSIEQVPISVEGRNLRGEKVKVRILGQETRNRAEKSRRVLQGEVDSFRPTNPGRSPGIGHSTHD

SEQ ID NO: 113 - protein, 81 aa, Solanum tuberosum
MASSYKKSIYMVLFYVFVFLLLQQCELIVASRVVVMKFHQPKPPSTNIFSFNRYKKSEVVKDYSGPGHSPGMGHDNPPGAS

SEQ ID NO: 114 - protein, 94 aa, Solanum tuberosum
MAILSYHKVICMFILYIFIISIALQQFVLVDASRSFSRYPPPPPPVEITHGEVKSLSSDNFSFNGSKSKYEKKDIPYVTPGHSPGMGHDTPPSS

SEQ ID NO: 115 - protein, 99 aa, Brassica napus
MGQKKTLFVCVFFVMVLFNGFNCVHGRTLRNMKVDDKMNVGHDDSKTMKAMNNDLIVDEKAVQLSQPPPSPPPESKDAEDFRPTTPGHSPGIGHSLSHN

SEQ ID NO: 116 - protein, 56 aa, Brassica napus
NVDGHKEGIEVFQAKILKNIYAYAPTDPGNSPGIGHHKMDVHVSNDFKVVRKLKKN

SEQ ID NO: 117 - protein, 188 aa, Brassica napus
MMTIMAISIVFVQVPSTTEARPLEITENKNHFKVTSLNNFVSTIPVGHNVDGHKEGIELFQEKILKNIYAYAPTDPGNSPGIGHHKMDVHAPELSNDFKVVRPLEITENKNHFKVMSLNNFVSTVPEGHNVDGHKEGIEVFQAKILKNIYAYAPTDPGNSPGIGHHKMDVHAPARSNDFKVVRKLKKN

SEQ ID NO: 118 - protein, 154 aa, Eucalyptus grandis
MLSRHLLSRVLSLSLSLSLSPPPLPPTVPAMAPNKVLYAFAFLLLALSLELQSTQARQLKLTMQKQKSFPNKLPNVHKLLEKELRKTIAEQSRNLHGEILNKATNAAVSTTPAPPPSSTIVAATTPPPSPGRSLDDFRPTQPGHSPGVGHSLQN

SEQ ID NO: 119 - protein, 83 aa, Mimulus guttatus
MAAKVFACLVFIFAILSNQVFHMEGRNLVVRENASNAEARGENIKSPNKEIGSHRFRFDEGYMDSYRPTTPGHSPGIGHSKHD

SEQ ID NO: 120 - protein, 92 aa, Aquilegia coerulea
MAKNKLICTCTLLLVLVLSHEMIHTEGRHLKIKKRTACVKCSSSNTVRGKKESDGQKTSDVHHKITPMAGFVEAFRPTTPGHSPGIGHSIQH

SEQ ID NO: 121 - protein, 190 aa, Oryza sativa
MKGKRTFLSSLNKEHIKKFYVLERVVAQFYLFSSQGRPLPDDDGITSEMQIRRYLLSHGNGVVEGAVSPSSEIGGPMVGASGGVRPTNPGHSPGIGHHVAINGDVDDDDVRPTNPGHSPGIGHHAIVNGADDADDVRPTNPGHSPGIGHAVVNSADDDADDVRPTNPGHSPGIGHAFVNKIDGPAGKKKL

SEQ ID NO: 122 - protein, 176 aa, Oryza sativa
MANICTMLAILVFSLQLFSSQGRPLPDDDGITSEMQIRRYLLSHGNGVVEGAVSPSSEIGGPMVGASGGVRPTNPGHSPGIGHHVVINGDVDDDDVRPTNPGHSPGIGHHAIVNGADDADDVRPTNPGHSPGIGHAVVNGADDNADDVRPTNPGHSPGIGHAFVNKIDGPAGKKKL

SEQ ID NO: 123 - protein, 102 aa, Oryza sativa
MALNKSSNSISKAFFLVLIILASQVMLSHGIPLEMHRRYLLSHAADATKGVMEGTITPTEGEGFAGANDDVRPTNPGHSPGIGHAFTNNKIGRKLLLAADDV

SEQ ID NO: 124 - protein, 137 aa, Oryza sativa
MSSSKLNLIFVLGIIFFLSSDMIIVCSQGRPLIAEAAAAAAAQQQRHLLSSSSSAPRSGGDVEEAAAGGGKGTTTAMTQGTLSPDAAESGGGGGGGVGIVEDARPTAPGHSPGAGHAFTNKNGVGRRLLVVTISTLI

SEQ ID NO: 125 - protein, 102 aa, Oryza sativa
MALNKSSNSISIAFFLVLIILASQVMLSHGIPLEMHRRYLLSHAADATKGVMEGTITPTEGEGFAGANDDVRPTNPGHSPGIGHAFTNNKIGRKLLLAADDV

SEQ ID NO: 126 - protein, 182 aa, Oryza barthii
MALNKNVSNICTMLAILVFSLQLFSSQGRPLPDDDGITSEMQIRRYLLSHGNRVVEGAVSPSSEIGGPMVGASGGVRPTNPGHSPGIGHHVVINGDIDDDDVRPTNPGHSPGIGHHAIVNGADDADDVRPTNPGHSPGIGHAVVNGADDDADDVRPTNPGHSPGIGHAFVNKIDGPAGKKKL

SEQ ID NO: 127 - protein, 113 aa, Sorghum bicolor
MALNKKNTNNTCTSVLLLLSLVIFSQFLASHGRPLPTGSYITTAAAVHGRNLLSHGSGSVPKGMLEGTVSPSSEIHGDNGSMVGADDVRPSNPGHSPGIGHAFINEKGTGRKL

SEQ ID NO: 128 - protein, 96 aa, Sorghum bicolor
MASSKVVCACILIILVISSQADARRLVTATCNGKEGACKGGVVVVEGYGGFSAKQKMATATSSEQVGEGMPATTTDSRPTAPGNSPGIGNRGKTNN

SEQ ID NO: 129 - protein, 93 aa, Triticum aestivum
MAGSKHASSCTCILIILVVSSHLAPCEARRLMVASAKITGDEACKSSGCRAVQGTASGAAATSKMATTDGRGTGPGHSPGIGNKLHAAGNDRR

SEQ ID NO: 130 - protein, 99 aa, Triticum aestivum
MAGSKHVSSCTCILIMLVVSSHLASCPCEARRLMAASAKINGDEACMSAGCRAVQGTASGTAEATWKMATTDSRGTAPGHSPGIGNKLHAAGTVTVKRN

SEQ ID NO: 131 - protein, 99 aa, Triticum aestivum
MARSKVLCTCILIIILSSIQAEARRLTTATAVTVASKGKEPWCALESNSRSLRATSSETSIAGAQGLNGGAMSTATTVESRGTAPGNSPGIGNKGKINN

SEQ ID NO: 132 - protein, 88 aa, Hordeum vulgare
FRPGAPATGGRRRRRRWSGGSSRRTSTRWAAAWCCARRTSRGRRCSWRPTTPGTSPGITSWWTAGSPWRSRSTCRRAEDGGEQPEENE

SEQ ID NO: 133 - protein, 71 aa, Hordeum vulgare
RHEASRRTSTRWAAAWCCARRTSRGRRCSWRPTTPGTSPGITSWWTAGSPWRSRSTCRRAEDGGEQPEENE

SEQ ID NO: 134 - protein, 75 aa, Hordeum vulgare
MAISSKIAVVFMLLLSTTFMQLPVPADARRLEVKAPILNVHRPCTGRSTLETPPEQVESTTPGHSPSIGHNSPPN

SEQ ID NO: 135 - protein, 96 aa, Saccharum officinarum
MASSKVVCACILIILVISSRADARRLVAATCNGKEGACKGGIIVVEGYGGFSAKQKMATARSTEEVSEGMPATTMDSRPTYPGNSPGIGNKGQINN

SEQ ID NO: 136 - protein, 34 aa, Saccharum officinarum
TMDSRPTYPGNSPGIGNKGENQQLAGRVLICVLI

SEQ ID NO: 137 - protein, 60 aa, Zea mays
SSHADARRLVATTCNGTEGGACKGGIFVQGYAGLSARQKMAATATSTEQVVGGGGEGMPA

SEQ ID NO: 138 - protein, 112 aa, Zea mays
MQQPYEHSGSSGSSPACRSRQIAPAHCTAPTTVASTPRSRYLLVVQSSSATTATAYSTTKAGMIEGTVTPSEGGAPGATEDVRPTNPSHSPGIGHAFTHNKIGRKLLAAISQ

SEQ ID NO: 139 - protein, 90 aa, Zea mays
AAGLVVKPSWACIVIIVLIVTLSSGAASGEARRLLMAEKHAAEGACAGGCSPPVQGLTATTTSKMATTDGRPTAPGHSPGIGNKIAGNTR

SEQ ID NO: 140 - protein, 92 aa, Zea mays
MHSHRSRHLKPRQREAAGGVFFNGGTEGGACKGGIFVQGYAGLSARQKMAATATSTEQVVVVGEGMPATTTDSRPTAPGNSPGIGNKGKINN

SEQ ID NO: 141 - protein, 113 aa, Brachypodium distachyon
MAPSISKNTNTCTCALLLIFVVLFSQLVESQSRSLPHGSLISTMHRRYLLSHVNGASPNGLAEGAVSPPSEIHGGDGPLVDVRDGVRPSNPGHSPGIGHSFVNRNGPAGNNKL

SEQ ID NO: 142 - protein, 109 aa, Setaria italica
MALNKNPSTCTSALLLLALLVTFSQLLASQGRPFPTVSYITTMHGRTLLSHGSDSVPKGMVEGTVSPSSEIHGDKGSMVDADDVRPSTPRHSPGIGHAFINKNGLGRKL

SEQ ID NO: 143 - protein, 135 aa, Phoenix dactilifera
MAGKKQFYSCILVIVLILANDYLSSEGRHLKEEKFKSRGCRECPERGDSKIERRTSSMVSNTIEGHDNRVLMVAMDARPTAGDSNIERGTSSMTSKTIEGHDARVLTAAIDSRPTAPGHSPGVGHSINSRGGDKN

SEQ ID NO: 144 - protein, 92 aa, Phoenix dactylifera
MVGIKPVHISALFVVLILARKFALTEERHFILVKTKISEKCPKQGDTRIGRMNRGINHGDAVLAFADGDRPSVPGHSPGVGHSHESKDGGKN

SEQ ID NO: 145 - protein, 115 aa, Phoenix dactylifera
MPNLPLSLSLSLSLMAGKKHFYACALVIVLILVNECLSSEGRHLMAGKFKAKGCEECLARGGNNIEGTTSSLVSHTIEGHDDRVLIVTTEDARPTTPGHSPGVGHGIKSSGGDKN

SEQ ID NO: 146 - protein, 99 aa, Phoenix dactylifera
MAANKRFYPCALLIIMVLASETFTSEGRTLMEDKARVCRRCLVENTSFKGLVEGPTVPPAVDGDNALMADTEDARPTTPGHSPGVGHSFNGKDVINKDV

SEQ ID NO: 147 - protein, 100 aa, Phoenix dactylifera
MAANKPFYTYALLILMILAFETFTSVGRTLVEDKTKVCRRCLVQDAGAKGMVEGPISPPAIHGDDALMVGISDARPTTPGHSPGVGHSFNYKNVVINKNV

SEQ ID NO: 148 - protein, 15 aa, Arabidopsis thaliana
DFRPTNPGNSPGVGH

SEQ ID NO: 149 - protein, 15 aa, Arabidopsis thaliana
DFAPTNPGDSPGIRH

SEQ ID NO: 150 - protein, 15 aa, Arabidopsis thaliana
TFRPTEPGHSPGIGH

SEQ ID NO: 151 - protein, 15 aa, Arabidopsis thaliana
AFRPTHQGPSQGIGH

SEQ ID NO: 152 - protein, 15 aa, Arabidopsis thaliana
DFVPTSPGNSPGVGH

SEQ ID NO: 153 - protein, 15 aa, Arabidopsis thaliana
DFAPTSPGHSPGVGH

SEQ ID NO: 154 - protein, 15 aa, Arabidopsis thaliana
DFAPTSPGNSPGIGH

SEQ ID NO: 155 - protein, 15 aa, Arabidopsis thaliana
DFKPTTPGHSPGVGH

SEQ ID NO: 156 - protein, 15 aa, Arabidopsis thaliana
DFRPTTPGHSPGIGH

SEQ ID NO: 157 - protein, 15 aa, Arabidopsis thaliana
DFGPTSPGNSPGIGH

SEQ ID NO: 158 - protein, 15 aa, Arabidopsis thaliana
DFEPTTPGHSPGVGH

SEQ ID NO: 159 - protein, 15 aa, Arabidopsis lyrata
DFRPTTPGHSPGIGH

SEQ ID NO: 160 - protein, 15 aa, Arabidopsis lyrata
TFRPTAPGHSPGIGH

SEQ ID NO: 161 - protein, 15 aa, Arabidopsis lyrata
DFAPTSPGNSPGVGH

SEQ ID NO: 162 - protein, 15 aa, Arabidopsis lyrata
DFAPTSPGHSPGVGH

SEQ ID NO: 163 - protein, 15 aa, Arabidopsis lyrata
EFAPTSPGNSPGIGH

SEQ ID NO: 164 - protein, 15 aa, Arabidopsis lyrata
DFAPTTPGNSPGMGH

SEQ ID NO: 165 - protein, 15 aa, Arabidopsis lyrata
DFKPTTPGHSPGVGH

SEQ ID NO: 166 - protein, 15 aa, Medicago truncatula
AFQPTTPGNSPGVGH

SEQ ID NO: 167 - protein, 15 aa, Medicago truncatula
EFQKTNPGHSPGVGH

SEQ ID NO: 168 - protein, 15 aa, Medicago truncatula
AFRPTTPGHSPGIGH

SEQ ID NO: 169 - protein, 15 aa, Medicago truncatula
DFRPTTPGNSPGAGH

SEQ ID NO: 170 - protein, 15 aa, Medicago truncatula
AFRPTSPGHSPGVGH

SEQ ID NO: 171 - protein, 15 aa, Medicago truncatula
AFRPTTPGHSPGVGH

SEQ ID NO: 172 - protein, 15 aa, Medicago truncatula
DFRPTSPGHSPGVGH

SEQ ID NO: 173 - protein, 15 aa, Medicago truncatula
SFRPTTPGSSPGVGH

SEQ ID NO: 174 - protein, 15 aa, Medicago truncatula
GFKPTNPSHSPGVGH

SEQ ID NO: 175 - protein, 15 aa, Medicago truncatula
AFRPTTPGNSPGVGH

SEQ ID NO: 176 - protein, 15 aa, Medicago truncatula
AFRPTTPGSSPGVGH

SEQ ID NO: 177 - protein, 15 aa, Medicago truncatula
AFKPTYPNHSPGVGH

SEQ ID NO: 178 - protein, 15 aa, Medicago truncatula
AFRPTPSGHSLGVGH

SEQ ID NO: 179 - protein, 15 aa, Medicago truncatula
AFRPTPPGHSPGGGH

SEQ ID NO: 180 - protein, 15 aa, Medicago truncatula
AFRPNPPGHSPGGGH

SEQ ID NO: 181 - protein, 15 aa, Medicago truncatula
AFRPTPPGHSPGGGH

SEQ ID NO: 182 - protein, 15 aa, Medicago truncatula
AFRPTSPGHSPGVGH

SEQ ID NO: 183 - protein, 15 aa, Solanum lycopersicum
ISEEGGPGHSPGVGH

SEQ ID NO: 184 - protein, 15 aa, Solanum lycopersicum
VKDYSGPGHSPGMGH

SEQ ID NO: 185 - protein, 15 aa, Solanum lycopersicum
VKDYSGPGHSPGMGH

SEQ ID NO: 186 - protein, 15 aa, Solanum lycopersicum
DFGPTGPGHSPGIGH

SEQ ID NO: 187 - protein, 15 aa, Gossypium hirsutum
AFRSTTPGHSPGAGH

SEQ ID NO: 188 - protein, 15 aa, Lactuca sativa
AFRPTTPGNSPGAGH

SEQ ID NO: 189 - protein, 15 aa, Lactuca sativa
GFRPTKPGNSPGAGH

SEQ ID NO: 190 - protein, 15 aa, Euphorbia esula
DFRPTSPGYSPGVGH

SEQ ID NO: 191 - protein, 15 aa, Euphorbia esula
DFRPTEPGYSPGVGH

SEQ ID NO: 192 - protein, 15 aa, Euphorbia esula
DYRPTEPGHSPGAGH

SEQ ID NO: 193 - protein, 15 aa, Euphorbia esula
DFRPTAPGFSPGVGH

SEQ ID NO: 194 - protein, 15 aa, Euphorbia esula
AFRPTAPGHSPGVGH

SEQ ID NO: 195 - protein, 15 aa, Euphorbia esula
AFRPTAPGHSPGVGH

SEQ ID NO: 196 - protein, 15 aa, Euphorbia esula
AFRPTAPGHSPGVGH

SEQ ID NO: 197 - protein, 15 aa, Euphorbia esula
AFRPTAPGHSPGVGY

SEQ ID NO: 198 - protein, 15 aa, Euphorbia esula
AFRPTAPGHSPGVGH

SEQ ID NO: 199 - protein, 15 aa, Glycine max
AFRPTTPGGSPGVGH

SEQ ID NO: 200 - protein, 15 aa, Glycine max
DFKPTDPGHSPGVGH

SEQ ID NO: 201 - protein, 15 aa, Glycine max
AFRPTTPGHSPGVGH

SEQ ID NO: 202 - protein, 15 aa, Glycine max
AFRPTTPGNSPGVGH

SEQ ID NO: 203 - protein, 15 aa, Glycine max
NFRPTAPGHSPGVGH

SEQ ID NO: 204 - protein, 15 aa, Glycine max
DFKPTDPGHSPGVGH

SEQ ID NO: 205 - protein, 15 aa, Glycine max
DFRPTTPGHSPGVGH

SEQ ID NO: 206 - protein, 15 aa, Glycine max
DFQPTDPGHSPGAGH

SEQ ID NO: 207 - protein, 15 aa, Glycine max
AFRPTTPGNSPGVGH

SEQ ID NO: 208 - protein, 15 aa, Glycine max
DFRPTDPGHSPGVGH

SEQ ID NO: 209 - protein, 15 aa, Glycine max
NFRPTAPGHSPGVGH

SEQ ID NO: 210 - protein, 15 aa, Glycine max
DFRPMDPGHSPGAGH

SEQ ID NO: 211 - protein, 15 aa, Glycine max
AFRPTCRGHSPGAGH

SEQ ID NO: 212 - protein, 15 aa, Glycine max
AFRPTTPGHSPGVGH

SEQ ID NO: 213 - protein, 15 aa, Glycine max
GFKPTNPGHSPGVGH

SEQ ID NO: 214 - protein, 15 aa, Glycine max
AFRPTTPGNSPGVGH

SEQ ID NO: 215 - protein, 15 aa, Glycine max
AFRPTTPGGSPGVGH

SEQ ID NO: 216 - protein, 15 aa, Glycine max
DFKPTDPGHSPGVGH

SEQ ID NO: 217 - protein, 15 aa, Glycine max
DFRPTDPGHSPGAGH

SEQ ID NO: 218 - protein, 15 aa, Lotus japonicus
DFKPTDPGHSPGVGH

SEQ ID NO: 219 - protein, 15 aa, Lotus japonicus
KQPTTGPGHSPGVGH

SEQ ID NO: 220 - protein, 15 aa, Lotus japonicus
AFRPTSPGHSPGVGH

SEQ ID NO: 221 - protein, 15 aa, Lotus japonicus
AFRPTTPGHSPGVGH

SEQ ID NO: 222 - protein, 15 aa, Lotus japonicus
AFRPTTPGHSPGVGH

SEQ ID NO: 223 - protein, 15 aa, Lotus japonicus
AFEPTTPGNSPGVGH

SEQ ID NO: 224 - protein, 15 aa, Lotus japonicus
DFKPTDPGHSPGVGH

SEQ ID NO: 225 - protein, 15 aa, Populus trichocarpa
DFRPTTPGVSPGVGH

SEQ ID NO: 226 - protein, 15 aa, Populus trichocarpa
DFQPTTPGHSPGAGH

SEQ ID NO: 227 - protein, 15 aa, Populus trichocarpa
AFKPTTPGHSPGAGH

SEQ ID NO: 228 - protein, 15 aa, Populus trichocarpa
AFKPTTPGHSPGIGH

SEQ ID NO: 229 - protein, 15 aa, Populus trichocarpa
DFRPTVPGHSPGIGH

SEQ ID NO: 230 - protein, 15 aa, Populus trichocarpa
AFRPPTPGHSPGVGH

SEQ ID NO: 231 - protein, 15 aa, Populus trichocarpa
DFKPITSGQSPGVGH

SEQ ID NO: 232 - protein, 15 aa, Populus trichocarpa
DFQPTTPGNSPGVGH

SEQ ID NO: 233 - protein, 15 aa, Populus trichocarpa
AFKPTTPGHSPGVGH

SEQ ID NO: 234 - protein, 15 aa, Populus trichocarpa
AFKPTTPGHSPGVGH

SEQ ID NO: 235 - protein, 15 aa, Populus trichocarpa
EHSVTTPGHSPAVGH

SEQ ID NO: 236 - protein, 15 aa, Populus trichocarpa
AFKPTTPGHSPGIGH

SEQ ID NO: 237 - protein, 15 aa, Populus trichocarpa
GFRPAVPIQGPGVGH

SEQ ID NO: 238 - protein, 15 aa, Populus trichocarpa
AFKPTTPGHSPGIGH

SEQ ID NO: 239 - protein, 15 aa, Populus trichocarpa
TFKPTTPGHSPGIGH

SEQ ID NO: 240 - protein, 15 aa, Populus trichocarpa
KHSVTTPGHSSRVGH

SEQ ID NO: 241 - protein, 15 aa, Populus trichocarpa
TFKPTTPGHSPGIGH

SEQ ID NO: 242 - protein, 15 aa, Populus trichocarpa
AFRPTTPGHSPGVGH

SEQ ID NO: 243 - protein, 15 aa, Populus trichocarpa
DFKPTTPGHSPGAGH

SEQ ID NO: 244 - protein, 15 aa, Populus trichocarpa
DFRPTAPGHSPGVGH

SEQ ID NO: 245 - protein, 15 aa, Vitis vinifera
GFRPTTPGSSPGIGH

SEQ ID NO: 246 - protein, 15 aa, Vitis vinifera
DFRPTTPGSSPGVGH

SEQ ID NO: 247 - protein, 15 aa, Vitis vinifera
DYRPTKPGHSPGVGH

SEQ ID NO: 248 - protein, 15 aa, Vitis vinifera
GFRPTTPGSSPGIGH

SEQ ID NO: 249 - protein, 15 aa, Vitis vinifera
DFRPTPPGSSPGIGH

SEQ ID NO: 250 - protein, 15 aa, Vitis vinifera
DFRPTTPGHSPGVGH

SEQ ID NO: 251 - protein, 15 aa, Vitis vinifera
DVQSTTPGHSPGVGH

SEQ ID NO: 252 - protein, 15 aa, Vitis vinifera
DFRPTPPGSSPGIGH

SEQ ID NO: 253 - protein, 15 aa, Vitis vinifera
DFRPTTPGSSPGVGH

SEQ ID NO: 254 - protein, 15 aa, Vitis vinifera
DYRPTKPGHSPGVGH

SEQ ID NO: 255 - protein, 15 aa, Vitis vinifera
DFRPTSPGHSPGVGH

SEQ ID NO: 256 - protein, 15 aa, Ricinus communis
AFRPTPPGNSPGVGH

SEQ ID NO: 257 - protein, 15 aa, Ricinus communis
DFRPTSPGYSPGVGH

SEQ ID NO: 258 - protein, 15 aa, Ricinus communis
DFRPTAPGFSPGVGH

SEQ ID NO: 259 - protein, 15 aa, Ricinus communis
DYRPTQPGHSPGVGH

SEQ ID NO: 260 - protein, 15 aa, Ricinus communis
DFRPTAPGHSPGVGH

SEQ ID NO: 261 - protein, 15 aa, Ricinus communis
AFRPTNPGHSPGVGH

SEQ ID NO: 262 - protein, 15 aa, Ricinus communis
DFRPTNPGHSPGAGH

SEQ ID NO: 263 - protein, 15 aa, Ricinus communis
DFRPTAPGHSPGVGH

SEQ ID NO: 264 - protein, 15 aa, Ricinus communis
AFRPTAPGHSSGVGH

SEQ ID NO: 265 - protein, 15 aa, Ricinus communis
AFRPTNPGHSPGVGH

SEQ ID NO: 266 - protein, 15 aa, Casuarina glauca
AFRPTSPGHSPGVGH

SEQ ID NO: 267 - protein, 15 aa, Jatropha curcas
AFRPTNPGHSPGVGH

SEQ ID NO: 268 - protein, 15 aa, Jatropha curcas
DFRPTTGGHSPGAGH

SEQ ID NO: 269 - protein, 15 aa, Jatropha curcas
DFRPTKPGYSPGVGH

SEQ ID NO: 270 - protein, 15 aa, Jatropha curcas
DFRPTSPGYSPGVGH

SEQ ID NO: 271 - protein, 15 aa, Jatropha curcas
DFRPTSPGYSPGVGH

SEQ ID NO: 272 - protein, 15 aa, Jatropha curcas
DFRPTAPGYSPGVGH

SEQ ID NO: 273 - protein, 15 aa, Theobroma cacao
DFRPTTPGHSPGAGH

SEQ ID NO: 274 - protein, 15 aa, Theobroma cacao
DFRPTPPGHSPGAGH

SEQ ID NO: 275 - protein, 15 aa, Theobroma cacao
DFRPPTPGHGPGAGH

SEQ ID NO: 276 - protein, 15 aa, Malus domestica
DYRPTTPGHSPGAGH

SEQ ID NO: 277 - protein, 15 aa, Carica papaya
GFRPTTPGISPGVGH

SEQ ID NO: 278 - protein, 15 aa, Carica papaya
AFRPTTPGHSPGVGH

SEQ ID NO: 279 - protein, 15 aa, Carica papaya
DFRPTTPGHSPGVGH

SEQ ID NO: 280 - protein, 15 aa, Fragaria vesca
DFRPTTPGNSPGAGH

SEQ ID NO: 281 - protein, 15 aa, Fragaria vesca
DYRPTQPGHSPGVGH

SEQ ID NO: 282 - protein, 15 aa, Fragaria vesca
AFRPTTPGHSPGVGH

SEQ ID NO: 283 - protein, 15 aa, Fragaria vesca
DFRPTTPGHSPGVGH

SEQ ID NO: 284 - protein, 15 aa, Prunus persica
AFRPTSPGHSPGVGH

SEQ ID NO: 285 - protein, 15 aa, Cucumis sativus
AFRPTSPGHSPGVGH

SEQ ID NO: 286 - protein, 15 aa, Manihot esculenta
CFRPTDPGNSPGVGH

SEQ ID NO: 287 - protein, 15 aa, Manihot esculenta
GFRPTTPGYSPGVGH

SEQ ID NO: 288 - protein, 15 aa, Manihot esculenta
DHRPTQPGHSPGVGH

SEQ ID NO: 289 - protein, 15 aa, Citrus sinensis
AFRPTSPGHSPGVGH

SEQ ID NO: 290 - protein, 15 aa, Citrus clementina
AFRPTSPGHSPGVGH

SEQ ID NO: 291 - protein, 15 aa, Catharanthus roseus
SFRPTNPGRSPGIGH

SEQ ID NO: 292 - protein, 15 aa, Solanum tuberosum
VKDYSGPGHSPGMGH

SEQ ID NO: 293 - protein, 15 aa, Solanum tuberosum
DIPYVTPGHSPGMGH

SEQ ID NO: 294 - protein, 15 aa, Brassica napus
DFRPTTPGHSPGIGH

SEQ ID NO: 295 - protein, 15 aa, Brassica napus
AYAPTDPGNSPGIGH

SEQ ID NO: 296 - protein, 15 aa, Brassica napus
AYAPTDPGNSPGIGH

SEQ ID NO: 297 - protein, 15 aa, Brassica napus
AYAPTDPGNSPGIGH

SEQ ID NO: 298 - protein, 15 aa, Eucalyptus grandis
DFRPTQPGHSPGVGH

SEQ ID NO: 299 - protein, 15 aa, Eucalyptus grandis
SYRPTTPGHSPGIGH

SEQ ID NO: 300 - protein, 15 aa, Aquilegia coerulea
AFRPTTPGHSPGIGH

SEQ ID NO: 301 - protein, 15 aa, Oryza sativa
GVRPTNPGHSPGIGH

SEQ ID NO: 302 - protein, 15 aa, Oryza sativa
DVRPTNPGHSPGIGH

SEQ ID NO: 303 - protein, 15 aa, Oryza sativa
DVRPTNPGHSPGIGH

SEQ ID NO: 304 - protein, 15 aa, Oryza sativa
DVRPTNPGHSPGIGH

SEQ ID NO: 305 - protein, 15 aa, Oryza sativa
GVRPTNPGHSPGIGH

SEQ ID NO: 306 - protein, 15 aa, Oryza sativa
DVRPTNPGHSPGIGH

SEQ ID NO: 307 - protein, 15 aa, Oryza sativa
DVRPTNPGHSPGIGH

SEQ ID NO: 308 - protein, 15 aa, Oryza sativa
DVRPTNPGHSPGIGH

SEQ ID NO: 309 - protein, 15 aa, Oryza sativa
DVRPTNPGHSPGIGH

SEQ ID NO: 310 - protein, 15 aa, Oryza sativa
DARPTAPGHSPGAGH

SEQ ID NO: 311 - protein, 15 aa, Oryza sativa
DVRPTNPGHSPGIGH

SEQ ID NO: 312 - protein, 15 aa, Oryza barthii
GVRPTNPGHSPGIGH

SEQ ID NO: 313 - protein, 15 aa, Oryza barthii
DVRPTNPGHSPGIGH

SEQ ID NO: 314 - protein, 15 aa, Oryza barthii
DVRPTNPGHSPGIGH

SEQ ID NO: 315 - protein, 15 aa, Sorghum bicolor
DVRPSNPGHSPGIGH

SEQ ID NO: 316 - protein, 15 aa, Sorghum bicolor
DSRPTAPGNSPGIGN

SEQ ID NO: 317 - protein, 15 aa, Triticum aestivum
DGRGTGPGHSPGIGN

SEQ ID NO: 318 - protein, 15 aa, Triticum aestivum
DSRGTAPGHSPGIGN

SEQ ID NO: 319 - protein, 15 aa, Triticum aestivum
DSRGTAPGHSPGIGN

SEQ ID NO: 320 - protein, 15 aa, Triticum aestivum
ESRGTAPGNSPGIGN

SEQ ID NO: 321 - protein, 15 aa, Hordeum vulgare
SWRPTTPGTSPGITS

SEQ ID NO: 322 - protein, 15 aa, Hordeum vulgare
SWRPTTPGTSPGITS

SEQ ID NO: 323 - protein, 15 aa, Hordeum vulgare
QVESTTPGHSPSIGH

SEQ ID NO: 324 - protein, 15 aa, Saccharum officinarum
DSRPTYPGNSPGIGN

SEQ ID NO: 325 - protein, 15 aa, Saccharum officinarum
DSRPTYPGNSPGIGN

SEQ ID NO: 326 - protein, 15 aa, Zea mays
DSRPTAPGNSPGIGN

SEQ ID NO: 327 - protein, 15 aa, Zea mays
DVRPTNPSHSPGIGH

SEQ ID NO: 328 - protein, 15 aa, Zea mays
DGRPTAPGHSPGIGN

SEQ ID NO: 329 - protein, 15 aa, Zea mays
DSRPTAPGNSPGIGN

SEQ ID NO: 330 - protein, 15 aa, Brachypodium distachyon
GVRPSNPGHSPGIGH

SEQ ID NO: 331 - protein, 15 aa, Setaria italica
DVRPSTPRHSPGIGH

SEQ ID NO: 332 - protein, 15 aa, Phoenix dactylifera
DSRPTAPGHSPGVGH

SEQ ID NO: 333 - protein, 15 aa, Phoenix dactylifera
GDRPSVPGHSPGVGH

SEQ ID NO: 334 - protein, 15 aa, Phoenix dactylifera
DARPTTPGHSPGVGH

SEQ ID NO: 335 - protein, 15 aa, Phoenix dactylifera
DARPTTPGHSPGVGH

SEQ ID NO: 336 - protein, 15 aa, Phoenix dactylifera
DARPTTPGHSPGVGH

SEQ ID NO: 337 - DNA, 2187 bp, Medicago truncatula
cttcaactttgacgatttagattcgatattttgacacaccaacaataaagaatacaaaactatcatcatagatataaaacattagagctagtaatgatgcattcaaaacccaatcaaattgtttatacatgcagtgtagtacaaaattttgctacaagtcaattcaatatttcaatttgagagaagaaacttatacaattcattattttttcttcaactttgagcatgcggtgcacccaatttgataattagcctactataaagtttaaacaagagaggaacccagaaaaagctgcctttcacttcaacttgtccatcaaaataaaatgctgaaattaaatagagaccataaactgcttaagtagtgcttaattatactataatcaatgaaattaattaaactataccactgttagtggaagggaaggtacaagagaatctcatatatacatatctttcttcatcaggggtccattctaaattaatggtgcgccagtccttcaactattctttattggttcatcataacttcaaatcttaccagttttccaaaggaacaaaattacaactacatatctcaactacttgctaaatgtccctttctcaaccttcatattaacctataccaagcacatatattaacctatactcatcggattagacgtgtcttactgttggatacatattgtgtctgacatcatcacatatgtttttttaacggaaaaatttattaaaaatcaagtccctaaagagagaccgacaagttagaaacaacataataaattattattgacacatatgattacattaaattgtataaatttttcaaataattatcgatgttgacgttctgtattgtgttccgtgtccgtgtcttagtccatacttcatacataataactattacaatgtatggccatgcaaaattgacaaaaaataattggccaactacattggagattcgtattgattgaacttaattttgtatggaatagagatacgattcacatgtattataggatgctgataataatcatgttttggacataaatagaatggaatagagaaaacaagagagcagaataaagcaaaacttccattagaggccacaattatttatattaaaaaataataattaattatgcttcaacatacattgtgatagattctcatcatttctgaaaagaattggaagatttcaacactaattttgagagtatatagtctaaaaaatttatacggtttacgattcagttatggttaagtattatttaaaaaactaacagaaatatgtttttttgtttaattttgttgaagataagtaaggatagattaaaatacaactaattatttttgtttcttgtagtttttgaaatcacttttaaatgagtttttgacaaaactgtgacaatgtaacaaaatacataaaaataatggaaaaatcacaagtttccgaaaatttgcaaaacaaacattaaagttcgatagaaaacatagaatactgaaccgtaagcaattgatccattgctcggcccataatctaaatatataaaataaatgtcaccattttaaaaaatgtatatatttatacaccaaaaacttaagtaatttgagctcaccatttcttatattattgagcatatctaagcgtaattgtgactgttccacttaaaatcaagatatttagatcttaaatcatatataataaggttgatatgcaatgcaacataagaagagaggtgaatatcaaaagagacatatatcatattcataataatgcaaatgactaaaaagagaaagatttttatatatgcacccccaaaagagcatgttcgcccacgtgagatagtgatgtgacccatagaattaaaaaatatactaatttgacatataaaattaaaagctagctatttttgacacataaacggtccaaatttgcagatcataacactccacaaattaagtcaaatgaaataattagcactaagaatcttataagagagcactgtcacactcacacacattctacattataaatacccctcaagatcccataacatttcatatcatatatcttctaatttgaactataacaagcttaaactttcaatacatatagttcattcattctctctactctaccttctctatttgttcgtgttaatggcttataaatttcaatacaca

SEQ ID NO: 338 - protein, 140 aa, Picea glauca
MKGYAMIVLLLLASRLGEAARIFGFKPFYLNSDSQVRAAPASSYPSVEEKTPEKAVLALNERLRRRASVEKHPGSETNLKPNLSAKSKASNQRSNGLLRTTFPSVKFDVVEADMEKTVAYPELLGKSPGVGHDIQPGSRH

SEQ ID NO: 339 - protein, 140 aa, Picea glauca
MKGYAMIVLLLLASRLGEAARIFGFKPFYLNSDSQVRAALASSYPSVEEKTPEKAVLALNERLRRRASVEKHPGSESNLKPNLSAKSKASNQRSNELLRTILPSVKFDAVEPDMEKTVAPFEPLGHSPGIGHDDPPRSRH

SEQ ID NO: 340 - protein, 141 aa, Picea glauca
MKGCAMIVLLFLAAPLGEAARILCFKLFLMNSDSQVKAAPARSYALVQERAPGKAVLELKERLSRKASREKYHGSEANMNPNISANSTASHQHSNGLLQKIHPSLKFDVVEPEREKSFTPFLPLLGHSPGVGHNNPPGFRH

SEQ ID NO: 341 - protein, 142 aa, Picea glauca
MMKGCAMIVLLFLAAPLGEASRILCFKLFLMNSDSQVKAAPARSYALVQERAPGKAVLELKERLSRKASREKYHGSEANMNPNISANSTASHQHSNGLLQKIHPSLKFDVVEPEREKSFTPFLPLLGHSPGVGHNNPPGFRH

SEQ ID NO: 342 - protein, 142 aa, Picea glauca
MMKGCAMIVLLFLAAPLGEASRILCFKLFLMNSDSQVKAAPARSYALVQERAPGKAVLELKERLSRKASREKYHGSEANMNPNISANSTASHQHSNGLLQKIHPSLKFDVVEPEREKSFTPFLPLLGHSPGIGHNNPPGFSH

SEQ ID NO: 343 - protein, 142 aa, Picea glauca
MMKGCAMIVLLFLAAPLREASRILCFKLFLMNSDSQVKAAPARSYALVQERAPGKAVLELKERLSRKASREKYHGSEANMNPNISANSTASHQHSNGLLQKIHPSLKFDVVEPEREKSFTPFLPLLGHSPGVGHNNPPGFRH

SEQ ID NO: 344 - protein, 136 aa, Picea glauca
MMKICAVIILLLFAAPLGEASRIFGFKPFSLKNDSQVKATTAVEESTAEKVVLEMNECFSKRANLEKHPGSEASLKPNVSAKSKASDQRSDELPQTILLSLKFNAVQHEEKKSVPPFQPLGHSPGIGHENPPGLRQ

SEQ ID NO: 345 - protein, 155 aa, Picea glauca
MKLGLWSVWGALMLSCVLSYSTAKARLMGFNPNAIQPPRPPALYKANEVGNIFRDTPMGRSSTIEKKQISIAPAETKLPSTLKVTVQGSLGHNDAHGIKEAETVAGGTQIFSKRPSESNNDSARMKKVDAVMAFRPSSSGHSPGIGHDDPPGPML

SEQ ID NO: 346 - protein, 73 aa, Pinus contorta
ADLEEHPGSDANFKPSVFVKSNASDQRSDGHVEELVPSLKFEVVQHDVQKTISPFKPLGHSPGIGHDDPPGSK

SEQ ID NO: 347 - protein, 74 aa, Pinus contorta
ADLEEHPGSDANFKPSVFVKSNASDQRFDGHVEELVPSLKFEVVQHDVQKTISPFKPLGHSPGIGHDDPPGSKH

SEQ ID NO: 348 - protein, 143 aa, Pinus contorta
MKTSVLIMLMFLAAPLVEAARIIGFKPFSLNRDSQVKATPATSYPLVEERAPAKVFVELKEPFGRRADLTDLEEHPGSDANFKPSVFVKSNASDQRSDGHVEELVPSLKFEVVQHDVQKTISPFKPLGHSPGIGHDDPPGSKH

SEQ ID NO: 349 - protein, 137 aa, Pinus contorta
IMLMFLAAPLVEAARIIGFKPFSLNRDSQVKATPATSYPLVEERAPAKVFVELKEPFGRRADLTDLEEHPGSDANFKPSVFVKSNASDQRFDGHVEELVPSLKFEVVQHDVQKTISPFKPLGHSPGIGHDDPPGSKH

SEQ ID NO: 350 - protein, 112 aa, Pinus engelmannii
MNSDSQVKATPARSYALVQERAPGKAVLELKERLSRKASREKHHGSEANMNPNISANSTASHQRSNGILQKIHSSLKFDVVEPEREKSFTPFLPLLGHSPGVGHNNPPGFKH

SEQ ID NO: 351 - protein, 15 aa, Picea glauca
VAYPELLGKSPGVGH

SEQ ID NO: 352 - protein, 15 aa, Picea glauca
VAPFEPLGHSPGIGH

SEQ ID NO: 353 - protein, 15 aa, Picea glauca
TPFLPLLGHSPGVGH

SEQ ID NO: 354 - protein, 15 aa, Picea glauca
TPFLPLLGHSPGVGH

SEQ ID NO: 355 - protein, 15 aa, Picea glauca
TPFLPLLGHSPGIGH

SEQ ID NO: 356 - protein, 15 aa, Picea glauca
TPFLPLLGHSPGVGH

SEQ ID NO: 357 - protein, 15 aa, Picea glauca
VPPFQPLGHSPGIGH

SEQ ID NO: 358 - protein, 15 aa, Picea glauca
AFRPSSSGHSPGIGH

SEQ ID NO: 359 - protein, 15 aa, Pinus contorta
ISPFKPLGHSPGIGH

SEQ ID NO: 360 - protein, 15 aa, Pinus contorta
ISPFKPLGHSPGIGH

SEQ ID NO: 361 - protein, 15 aa, Pinus contorta
ISPFKPLGHSPGIGH

SEQ ID NO: 362 - protein, 15 aa, Pinus contorta
ISPFKPLGHSPGIGH

SEQ ID NO: 363 - protein, 15 aa, Pinus engelmannii
TPFLPLLGHSPGVGH

SEQ ID NO: 364 - DNA, 201 bp, Meloidogyne hapla
atgattaatattaattcaattagattttttattatttttataattaattttatgatttatcaagtaatggctgttaataattcagctaatgacttccgaccaacaaacccaggccattcaccaggaattggacattgtaatttaattttatattttattggcaaaataatatatcaaaaaataagattagtcagaaaataa

SEQ ID NO: 365 - protein, 66 aa, Meloidogyne hapla
MININSIRFFIIFIINFMIYQVMAVNNSANDFRPTNPGHSPGIGHCNLILYFIGKIIYQKIRLVRK

SEQ ID NO: 366 - DNA, 183 bp, Meloidogyne hapla
atgattaaaattaattctattatattttttattatttttataattaattttatgatttatcaaataatggctgctaataattcagttgatgccttccgaccaacagccccaggccattcacccggagttggacattgtaatttaattttaaattttatatacaaaattaaatataaaaaataa

SEQ ID NO: 367 - protein, 60 aa, Meloidogyne hapla
MIKINSIIFFIIFIINFMIYQIMAANNSVDAFRPTAPGHSPGVGHCNLILNFIYKIKYKK

SEQ ID NO: 368 - DNA, 183 bp, Meloidogyne hapla
atgattaatattaattttattatattttttattatttttattattaattttatgatttatttcacaatggctggttaccaaccaacaaacccaggccattcacccggaattggccattgtaatgaattatctcaaaaaagattagggagtaataatttatcagatctgattagggttttttag

SEQ ID NO: 369 - protein, 60 aa, Meloidogyne hapla
MININFIIFFIIFIINFMIYFTMAGYQPTNPGHSPGIGHCNELSQKRLGSNNLSDLIRVF

SEQ ID NO: 370 - DNA, 213 bp, Meloidogyne hapla
atgactaaaattaattctattatattttttattatttttattattaattttatgatttattacaatttggctgataatgataaaccagccaaaattccacctttcaaaacagtcccaggccagagttcacctggagtagggcatggaattccaaatggaggtccacctggagttggacattgtgatttaattaaatttgatttacaaaattaa

SEQ ID NO: 371 - protein, 70 aa, Meloidogyne hapla
MTKINSIIFFIIFIINFMIYYNLADNDKPAKIPPFKTVPGQSSPGVGHGIPNGGPPGVGHCDLIKFDLQN

SEQ ID NO: 372 - DNA, 133 bp, Meloidogyne hapla
atgactaaaattaattctattatattttttattctttttataattaattttatgatttatcacataatggcagataatgttattaaaccagcatgcattggtaattcacctggagttggacattgtaattgaa

SEQ ID NO: 373 - protein, 43 aa, Meloidogyne hapla
MTKINSIIFFILFIINFMIYHIMADNVIKPACIGNSPGVGHCN

SEQ ID NO: 374 - DNA, 171 bp, Meloidogyne hapla
atgattaatattaattctattttattttttattttttttataattaattttatgatttatttcactatggctgccttccgaccaacaaatccaggcccttcacccgcaattggacatggaattccaaatggagttccacaacctccacccgtaaatggacattgtaattaa

SEQ ID NO: 375 - protein, 56 aa, Meloidogyne hapla
MININSILFFIFFIINFMIYFTMAAFRPTNPGPSPAIGHGIPNGVPQPPPVNGHCN

SEQ ID NO: 376 - DNA, 153 bp, Meloidogyne hapla
atgcctaaaattaattctattttattttttattctttttattattaattttatgatttatttcacaatggctggattccgaccaacaaatccaggcaattcacccggagctggacatggagctccaaatggaccccaaagtctccacccgtaa

SEQ ID NO: 377 - protein, 50 aa, Meloidogyne hapla
MPKINSILFFILFIINFMIYFTMAGFRPTNPGNSPGAGHGAPNGPQSLHP

SEQ ID NO: 378 - DNA, 168 bp, Meloidogyne hapla
atgactaaaattaattctattatattttttattatttttataattaattttatgatttatcaaataatggccgctaataagtcatgtaataccttccgacccacagctccgggccattcacccggaattggaaattgtagtttaattaaattttatttacaaaattaa

SEQ ID NO: 379 - protein, 55 aa, Meloidogyne hapla
MTKINSIIFFIIFIINFMIYQIMAANKSCNTFRPTAPGHSPGIGNCSLIKFYLQN

SEQ ID NO: 380 - DNA, 177 bp, Meloidogyne hapla
atgactaaaattaattctattatattttttattatttttattattaattttatgatttatcaaataatagcacctcaacctcctttctgcacaggaccaggccattcacctggagttggacatggaattccaaatggacttccatgtaagccaccagtaaatggacaatgtaattaa

SEQ ID NO: 381 - protein, 58 aa, Meloidogyne hapla
MTKINSIIFFIIFIINFMIYQIIAPQPPFCTGPGHSPGVGHGIPNGLPCKPPVNGQCN

SEQ ID NO: 382 - DNA, 212 bp, Meloidogyne hapla
atgactaaaattaactttctatttatatttttttatttatttttgattattaattttatgatttatcaaataatagcacctcaacctcctttctgcacaggatcaggccattcacccggagttggacatggaattccaaatggacttccatgtaagccaccagtaaatggacattgtaatttaattaaattttatttgcaaaattatataat

SEQ ID NO: 383 - protein, 70 aa, Meloidogyne hapla
MTKINFLFIFFYLFLIINFMIYQIIAPQPPFCTGSGHSPGVGHGIPNGLPCKPPVNGHCNLIKFYLQNYI

SEQ ID NO: 384 - DNA, 183 bp, Meloidogyne hapla
atgactaaaattaattctattatatttttattattttttataattaattttatgatttatcaaataatggctgttaataattcagttgatgccttccgaccaacagccccaggccattcacccggagttggacattgtaatttaattttaaaatttattgccaaaattaaatctctcaaataa

SEQ ID NO: 385 - protein, 60 aa, Meloidogyne hapla
MTKINSIIFLLFFIINFMIYQIMAVNNSVDAFRPTAPGHSPGVGHCNLILKFIAKIKSLK

SEQ ID NO: 386 - DNA, 135 bp, Meloidogyne hapla
atgattaatattaattctattatattttttattattttaataattaattttatgatttatttgacaatggcaggcaatccacctttccatactggcactggccgttcacccggagctggccatcattgtatttaa

SEQ ID NO: 387 - protein, 44 aa, Meloidogyne hapla
MININSIIFFIILIINFMIYLTMAGNPPFHTGTGRSPGAGHHCI

SEQ ID NO: 388 - protein, 39 aa, Meloidogyne incognita
MTKINSIIFLIFLIINFMNYYIIADVHPNNPGHSPGIGH

SEQ ID NO: 389 - protein, 39 aa, Meloidogyne incognita
MTKINSIIFLIFLIINFMNYYIMASRPTGPGHSPGVGNS

SEQ ID NO: 390 - protein, 39 aa, Meloidogyne incognita
MTKINSIIFLIFLIINFMNYYIMAAFRPTNPGHSPGVGH

SEQ ID NO: 391 - protein, 39 aa, Meloidogyne incognita
MTKINSIIFLIFLIINFMNYYIIAEVHPNNPGHSPGIGH

SEQ ID NO: 392 - protein, 39 aa, Meloidogyne incognita
MTKINSIIFLIFLIINFMNYYIIASRPTQPGHSPGVGNG

SEQ ID NO: 393 - protein, 57 aa, Meloidogyne incognita
MTKINSIIFLIILIINFMNYYIVAGTRATEPGHSPGAGHDAPNVAAHGAHGHGGPGK

SEQ ID NO: 394 - protein, 57 aa, Meloidogyne incognita
MTKINSIIFLIFLIINFMNYYIMAGTRPTEPGHSPGAGHDAPNVAAYGAHGHGPENK

SEQ ID NO: 395 - protein, 43 aa, Meloidogyne incognita
MIKINSKIIFLLFLLIIFMIYYTMAPAPPGRNTAPGHSPGIGH

SEQ ID NO: 396 - protein, 15 aa, Meloidogyne hapla
DFRPTNPGHSPGIGH

SEQ ID NO: 397 - protein, 15 aa, Meloidogyne hapla
AFRPTAPGHSPGVGH

SEQ ID NO: 398 - protein, 15 aa, Meloidogyne hapla
GYQPTNPGHSPGIGH

SEQ ID NO: 399 - protein, 15 aa, Meloidogyne hapla
PFKTVPGQSSPGVGH

SEQ ID NO: 400 - protein, 15 aa, Meloidogyne hapla
VIKPACIGNSPGVGH

SEQ ID NO: 401 - protein, 15 aa, Meloidogyne hapla
AFRPTNPGPSPAIGH

SEQ ID NO: 402 - protein, 15 aa, Meloidogyne hapla
GFRPTNPGNSPGAGH

SEQ ID NO: 403 - protein, 15 aa, Meloidogyne hapla
TFRPTAPGHSPGIGN

SEQ ID NO: 404 - protein, 15 aa, Meloidogyne hapla
PPFCTGPGHSPGVGH

SEQ ID NO: 405 - protein, 15 aa, Meloidogyne hapla
PPFCTGSGHSPGVGH

SEQ ID NO: 406 - protein, 15 aa, Meloidogyne hapla
AFRPTAPGHSPGVGH

SEQ ID NO: 407 - protein, 15 aa, Meloidogyne hapla
PPFHTGTGRSPGAGH

SEQ ID NO: 408 - protein, 15 aa, Meloidogyne incognita
DVHPNNPGHSPGIGH

SEQ ID NO: 409 - protein, 15 aa, Meloidogyne incognita
ASRPTGPGHSPGVGN

SEQ ID NO: 410 - protein, 15 aa, Meloidogyne incognita
AFRPTNPGHSPGVGH

SEQ ID NO: 411 - protein, 15 aa, Meloidogyne incognita
EVHPNNPGHSPGIGH

SEQ ID NO: 412 - protein, 15 aa, Meloidogyne incognita
SRPTQPGHSPGVGNG

SEQ ID NO: 413 - protein, 15 aa, Meloidogyne incognita
GTRATEPGHSPGAGH

SEQ ID NO: 414 - protein, 15 aa, Meloidogyne incognita
GTRPTEPGHSPGAGH

SEQ ID NO: 415 - protein, 15 aa, Meloidogyne incognita
PGRNTAPGHSPGIGH

SEQ ID NO: 416 - protein, 53 aa, Meloidogyne hapla
MKKMIKFINDKKAKYFSLNEDYTGPKHHGPRNNDKDTSSHKNVNKGKLQTKKP

SEQ ID NO: 417 - protein, 68 aa, Meloidogyne incognita
MKSTTFQFIFLLIIGIQLIYNLNNVDGANEQEINVDKIKGNNNGDGGSLLNEEDDYSAPNKKPPIHNP

SEQ ID NO: 418 - protein, 65 aa, Meloidogyne hapla
MFKLISLIIIAFLFLAGSIICPKGVHIPPSGPSKDIPCHVDPYGKECKDFCKNNRNDKLCKKGGR

SEQ ID NO: 419 - DNA, 681 bp, Meloidogyne hapla
atgaagatccctgttgccgcctggctccgcgagaaccggagcttcctgctgttcgtggctggtttcctgttcttccgcacggcgatcgcggattggaatccggtgcccagcggctcgatgcggccgacgatcctggaaggcgacgtcgtcttcgtcgatcggaccgcctatgactggaaagtgccgctgaccgatctgtcgctgcgccgcgtgggcgagccccggcgcggggatgtggtgaccttcagctcgccgcgcgacggcacgcggctgatcaagcgcgtggtggcggtgcccggcgacgtggtcgagctgcgtggcgaccgactgatcctcaacggcgaggcggcgacgtacgcgccggtggaggaggtcgcggaggcactggtccccggggtcgaggtgcgcggggtgcgcgaggtcgagcaatggggcggtcaggagcgcatcgtgcagttcctgcccgcgctgaacgcgccgcgcgatttcggtccggtggtggtgcccgccgatgcgttcttcatgatgggcgacaaccgggacaacagccaggactcgcgctacatcgggctggtgccgcgccggctgatcatcgggcaggcgcatcgggtgctggtgtcggcggacatcaaggggcgctggacgccgagatgggatcgtttcgccgcgcccttgaagtga

SEQ ID NO: 420 - protein, 226 aa, Meloidogyne hapla
MKIPVAAWLRENRSFLLFVAGFLFFRTAIADWNPVPSGSMRPTILEGDVVFVDRTAYDWKVPLTDLSLRRVGEPRRGDVVTFSSPRDGTRLIKRVVAVPGDVVELRGDRLILNGEAATYAPVEEVAEALVPGVEVRGVREVEQWGGQERIVQFLPALNAPRDFGPVVVPADAFFMMGDNRDNSQDSRYIGLVPRRLIIGQAHRVLVSADIKGRWTPRWDRFAAPLK

SEQ ID NO: 421 - protein, 15 aa, Artificial Sequence
  Synthetic Construct
XFRPTXPGXSPGXGX

SEQ ID NO: 422 - DNA, 32 bp, Medicago truncatula
caccatggcttataaatttcaatacacaatga

SEQ ID NO: 423 - DNA, 23 bp, Medicago truncatula
tcaatttccaattttgttttggt

SEQ ID NO: 424 - DNA, 22 bp, Medicago truncatula
ccgatgaagatatcgacgtgaa

SEQ ID NO: 425 - DNA, 29 bp, Medicago truncatula
gaactcatttgtagtatcctcagtcacat

SEQ ID NO: 426 - DNA, 20 bp, Medicago truncatula
tagctcgcatttgcttgttc

SEQ ID NO: 427 - DNA, 20 bp, Medicago truncatula
ggctgaatgctttgtctcaa

SEQ ID NO: 428 - DNA, 20 bp, Medicago truncatula
acgttgagctccaccatttt

SEQ ID NO: 429 - DNA, 20 bp, Medicago truncatula
gagcgctccacctcctatta

SEQ ID NO: 430 - DNA, 20 bp, Medicago truncatula
catggaggtggtgtttgatg

SEQ ID NO: 431 - DNA, 20 bp, Medicago truncatula
ttttcgccctacaagtccag

SEQ ID NO: 432 - DNA, 20 bp, Medicago truncatula
gtgttgttttgagcccaagg

SEQ ID NO: 433 - DNA, 20 bp, Medicago truncatula
tgttggtcgaaaagcttcaa

SEQ ID NO: 434 - DNA, 20 bp, Medicago truncatula
gctcatcatggagggaagtc

SEQ ID NO: 435 - DNA, 20 bp, Medicago truncatula
tatgccctggagatgtaggc

SEQ ID NO: 436 - DNA, 20 bp, Medicago truncatula
ccggatgttgaggtttttgt

SEQ ID NO: 437 - DNA, 20 bp, Medicago truncatula
ggccaactccaggactatga

SEQ ID NO: 438 - DNA, 20 bp, Medicago truncatula
tccaacaatattgccaccaa

SEQ ID NO: 439 - DNA, 20 bp, Medicago truncatula
gggttgtgggtctaaaagca

SEQ ID NO: 440 - DNA, 20 bp, Medicago truncatula
tgatgccaaatcatggtgtc

SEQ ID NO: 441 - DNA, 20 bp, Medicago truncatula
ggactgcttcctggtgttgt

SEQ ID NO: 442 - DNA, 21 bp, Medicago truncatula
tcaatggaagcatcaaggttt

SEQ ID NO: 443 - DNA, 20 bp, Medicago truncatula
tatatgtcccaccccaagac

SEQ ID NO: 444 - DNA, 20 bp, Medicago truncatula
agctccttccattggctttt

SEQ ID NO: 445 - DNA, 19 bp, Medicago truncatula
ccccaccaggactatgacc

SEQ ID NO: 446 - DNA, 21 bp, Medicago truncatula
ggagaaggagaaagggtctca

SEQ ID NO: 447 - DNA, 21 bp, Medicago truncatula
tcagaaggcctagttgaaatg

SEQ ID NO: 448 - DNA, 19 bp, Medicago truncatula
gaaccgaagggaagcataa

SEQ ID NO: 449 - DNA, 20 bp, Medicago truncatula
tgtcgtgccatacacctttt

SEQ ID NO: 450 - DNA, 20 bp, Medicago truncatula
gccacgctactgttttcgta

SEQ ID NO: 451 - DNA, 20 bp, Medicago truncatula
gagctggtctctgtggttca

SEQ ID NO: 452 - DNA, 22 bp, Meloidogyne hapla
gcacctcaacctcctttctgca

SEQ ID NO: 453 - DNA, 26 bp, Meloidogyne hapla
tgtccatttactggtggcttacatgg

SEQ ID NO: 454 - DNA, 21 bp, Medicago truncatula
aacttgttgcatgggtcttga

SEQ ID NO: 455 - DNA, 30 bp, Medicago truncatula
cattaagtttgacaaagagaaagagacaga

SEQ ID NO: 456 - protein, 15 aa, Medicago truncatula
  hydroxyproline (4)..(4)
  hydroxyproline (11)..(11)
AFQPTTPGNSPGVGH

SEQ ID NO: 457 - protein, 15 aa, Medicago truncatula
  hydroxyproline (11)..(11)
EFQKTNPGHSPGVGH

SEQ ID NO: 458 - protein, 19 aa, Meloidogyne hapla
  hydroxyproline (4)..(4)
  hydroxyproline (11)..(11)
AFRHYPTAPGHSHYPGVGH

SEQ ID NO: 459 - protein, 14 aa, Artificial Sequence
  Synthetic Construct
XXXXXPGXSPGXGX

SEQ ID NO: 460 - protein, 13 aa, Meloidogyne hapla
DYTGPKHHGPRNN

SEQ ID NO: 461 - protein, 14 aa, Meloidogyne incognita
DYSAPNKKPPIHNP

SEQ ID NO: 462 - protein, 13 aa, Selaginella moellendorffii
DYTPVHRKPPINN

SEQ ID NO: 463 - protein, 14 aa, Artificial Sequence
  Synthetic Construct
DYSTPGDEPDRQKN

SEQ ID NO: 464 - protein, 116 aa, Arabidopsis thaliana
MVSIRVICYLLVFSVLQVHAKVSNANFNSQAPQMKNSEGLGASNGTQIAKKHAEDVIENRKTLKHVNVKVEANEKNGLEIESKEMVKKRKNKKRLTKTESLTADYSNPGHHPPRHN

SEQ ID NO: 465 - protein, 109 aa, Arabidopsis thaliana
MTNITSSFLCLLILLLFCLSFGYSLHGDKDEVLSVDVGSNAKVMKHLDGDDAMKKAQVRGRSGQEFSKETTKMMMKKTTKKETNVEEEDDLVAYTADYWKPRHHPPKNN

SEQ ID NO: 466 - protein, 110 aa, Arabidopsis thaliana
MTTLSKILCVLIILLLCSFFRYSLHEDGNQQSSRDFVSTAKAIKYGDVMKKMIRGRKLMMASGEKEEAETKMKRGNRETERNSSKSVEEDGLVAYTADYWRAKHHPPKNN

SEQ ID NO: 467 - protein, 163 aa, Arabidopsis thaliana
MMRFTIIVIAFLLIIQSLEEEHILVYAHEGGEAGHKSLDYQGDQDSSTLHPKELFDAPRKVRFGRTTRAEKEQVTAMNNDSWSFKISGEHKQTNILADHDTTKNTFCKKMMIIVNDLTSLPTLEPSTSTNDMEKLARLLRDDYPIYSKPRRKPPVNNRAPDKF

SEQ ID NO: 468 - protein, 88 aa, Arabidopsis thaliana
MSSIHVASMILLLFLFLHHSDSRHLDNVHITASRFSLVKDQNVVSSSTSKEPVKVSRFVPGPLKHHHRRPPLLFADYPKPSTRPPRHN

SEQ ID NO: 469 - protein, 86 aa, Arabidopsis thaliana
MSCSLRSGLVIVFCFILLLLSSNVGCASAARRLRSHKHHHHKVASLDVFNGGERRRALGGVETGEEVVVMDYPQPHRKPPIHNEKS

SEQ ID NO: 470 - protein, 102 aa, Arabidopsis thaliana
MEMKKWSYANLITLALLFLFFIILLLAFQGGSRDDDHQHVHVAIRTKDISMGRKLKSLKPINPTKKNGFEYPDQGSHDVQEREVYVELRDYGQRKYKPPVHN

SEQ ID NO: 471 - protein, 123 aa, Arabidopsis thaliana
MKLIRVTLFLCALAILLLVTPTSSLQLKHPYSSPSQGLSKKIVTKMATRKLMIISSEYVMTSTSHEGSSEQLRVTSSGKSKDEEKKLSEEEEEKKALAKYLSMDYRTFRRRRPVHNKALPLDP

SEQ ID NO: 472 - protein, 79 aa, Arabidopsis thaliana
MAIRVSHKSFLVALLLILFISSPTQARSLREVVRNRTLLVVEKSQESRKIRHEGGGSDVDGLMDMDYNSANKKRPIHNR

SEQ ID NO: 473 - protein, 53 aa, Meloidogyne hapla; identical to SEQ ID NO: 416

SEQ ID NO: 474 - protein, 68 aa, Meloidogyne incognita; identical to SEQ ID NO: 417

SEQ ID NO: 475 - protein, 13 aa, Arabidopsis thaliana
DYSNPGHHPPRHN

SEQ ID NO: 476 - protein, 13 aa, Arabidopsis thaliana
DYWKPRHHPPKNN

SEQ ID NO: 477 - protein, 13 aa, Arabidopsis thaliana
DYWRAKHHPPKNN

SEQ ID NO: 478 - protein, 15 aa, Arabidopsis thaliana
DYPIYSKPRRKPPVN

SEQ ID NO: 479 - protein, 13 aa, Arabidopsis thaliana
DYPKPSTRPPRHN

SEQ ID NO: 480 - protein, 13 aa, Arabidopsis thaliana
DYPQPHRKPPIHN

SEQ ID NO: 481 - protein, 13 aa, Arabidopsis thaliana
DYGQRKYKPPVHN

SEQ ID NO: 482 - protein, 13 aa, Arabidopsis thaliana
DYRTFRRRRPVHN

SEQ ID NO: 483 - protein, 13 aa, Arabidopsis thaliana
DYNSANKKRPIHN

SEQ ID NO: 484 - protein, 77 aa, Arabidopsis thaliana
MAPCRTMMVLLCFVLFLAASSSCVAAARIGATMEMKKNIKRLTFKNSHIFGYLPKGVPIPPSAPSKRHNSFVNSLPH

SEQ ID NO: 485 - protein, 86 aa, Arabidopsis thaliana
MNLSHKTMFMTLYIVFLLIFGSYNATARIGPIKLSETEIVQTRSRQEIIGGFTFKGRVFHSFSKRVLVPPSGPSMRHNSVVNNLKH

SEQ ID NO: 486 - protein, 95 aa, Arabidopsis thaliana
MSSRNQRSRITSSFFVSFFTRTILLLLILLLGFCNGARTNTNVFNSKPHKKHNDAVSSSTKQFLGFLPRHFPVPASGPSRKHNDIGLLSWHRSSP

SEQ ID NO: 487 - protein, 99 aa, Arabidopsis thaliana
MSSRSHRSRKYQLTRTIPILVLLLVLLSCCNGARTTNVFNTSSPPKQKDVVSPPHDHVHHQVQDHKSVQFLGSLPRQFPVPTSGPSRKHNEIGLSSTKT

SEQ ID NO: 488 - protein, 93 aa, Arabidopsis thaliana
MYPTRPHYWRRRLSINRPQAFLLLILCLFFIHHCDASRFSSSSVFYRNPNYDHSNNTVRRGHFLGFLPRHLPVPASAPSRKHNDIGIQALLSP

SEQ ID NO: 489 - protein, 111 aa, Arabidopsis thaliana
MGNKRIKAMMILVVMIMMVFSWRICEADSLRRYSSSSRPQRFFKVRRPNPRNHHHQNQGFNGDDYPPESFSGFLPKTLPIPHSAPSRKHNVYGLQRTNSRSSTFWTHAHLS

SEQ ID NO: 490 - protein, 20 aa, Arabidopsis thaliana
FGYLPKGVPIPPSAPSKRHN

SEQ ID NO: 491 - protein, 21 aa, Arabidopsis thaliana
VFHSFSKRVLVPPSGPSMRHN

SEQ ID NO: 492 - protein, 21 aa, Arabidopsis thaliana
FLGFLPRHFPVPASGPSRKHN

SEQ ID NO: 493 - protein, 21 aa, Arabidopsis thaliana
FLGSLPRQFPVPTSGPSRKHN

SEQ ID NO: 494 - protein, 21 aa, Arabidopsis thaliana
FLGFLPRHLPVPASAPSRKHN

SEQ ID NO: 495 - protein, 21 aa, Arabidopsis thaliana
FSGFLPKTLPIPHSAPSRKHN

SEQ ID NO: 496 - protein, 17 aa, Meloidogyne hapla
CPKGVHIPPSGPSKDIP